(19) United States
(12) Patent Application Publication
(10) Pub. No.: US 2008/0019970 A1
(43) Pub. Date: Jan. 24, 2008

(54) METHODS FOR PREVENTING, POSTPONING OR IMPROVING THE OUTCOME OF SPINAL DEVICE AND FUSION PROCEDURES

(76) Inventor: James R. Gorman, Natick, MA (US)

Correspondence Address: FISH & RICHARDSON P.C., PO BOX 1022, MINNEAPOLIS, MN, 55440-1022, US

(21) Appl. No.: 11/775,189

(22) Filed: Jul. 9, 2007

Related U.S. Application Data
(60) Provisional application No. 60/847,493, filed on Sep. 27, 2006.
(60) Provisional application No. 60/819,555, filed on Jul. 7, 2006.

(51) Int. Cl. A61K 39/395; A61B 17/70; A61F 2/44; A61P 19/02; A61K 31/4164; A61K 31/435

(52) U.S. Cl. 424/141.1; 514/352; 514/401; 604/181; 604/31; 604/523; 606/61; 623/17.11

(57) ABSTRACT

Methods for identifying subjects who could benefit therapeutically from administration of a high specificity cytokine inhibitor are provided. Subjects that are identified include those that are eligible, based on pre-determined criteria, for a spinal device or fusion procedure, such as the implantation of a nucleus replacement device, an annular repair device, or a fusion device. Methods of preventing such procedures or improving the outcome of such procedures are also provided, and include administering a TAT to the subject by any route or regimen of administration, including the regimens described herein.

### CROSS-REFERENCE TO RELATED APPLICATIONS

This application claims priority under 35 U.S.C. §119 to U.S. Provisional Application Ser. Nos. 60/819,555, filed Jul. 7, 2006; and 60/847,493, filed Sep. 27, 2006, the contents of which are incorporated herein in their entireties.

This application is related to U.S. application Ser. No. ______ (Attorney Docket No. 21782-005001) and Ser. No. ______ (Attorney Docket No. 21782-007001), both filed concurrently herewith on Jul. 9, 2007, the entire contents of which are incorporated by reference herein in their entirety.

### TECHNICAL FIELD

This disclosure is related to identifying subjects who are currently treated preferentially by an invasive spinal procedure involving implantation of a device or fusion of vertebrae, but who, contrary to current teaching and practice, are surprisingly likely to benefit from treatment with a targeted anti-inflammatory therapy (TAT), such as an inflammatory cytokine inhibitor (IC-I), or an inflammatory mediator inhibitor (IM-I). Spinal device or fusion procedures include procedures that implant devices, such as nucleus replacement, annular repair, disk replacement, dynamic stabilization, or placement of anti-adhesion devices, or procedures in which two or more adjacent vertebrae are fused. The disclosure also relates to methods for preventing, reducing, postponing, delaying or eliminating the need for spinal device or fusion procedures, and also to methods for improving the therapeutic outcome of these procedures in the same patients. More particularly, this disclosure relates to the use of TATs, including TNF-α (TNF) inhibitors (TNF-Is), administered either by known or novel regimens, in subjects who have met the eligibility criteria in at least one predetermined standard of eligibility (SOE) for a spinal device or fusion procedure. Typically, the subject will meet the clinical criteria of eligibility for the spinal device or fusion procedure according to a skilled practitioner, and often according to the clinical eligibility criteria in a clinical practice guideline (CPG) or a clinical trial of the procedure. Such clinical eligibility criteria will usually include confirmation of a spinal disorder by appropriate imaging procedures such as MRI or CT, the presence of moderate to severe persistent symptoms such as radicular pain persisting for 6 weeks or more, and the failure to respond to conventional non-invasive, and in some cases, invasive, therapies.

### BACKGROUND

Inflammatory Cytokines (ICs) and Inflammatory Mediators (IMs)

ICs and/or IMs are implicated as causing, contributing to, exacerbating, or perpetuating the pathophysiology of a wide range of prevalent and troublesome diseases and disorders. New classes of TATs, including protein therapeutics, offer new possibilities of targeted therapy, and also limitations. For example, patients with spinal disorders, once they are identified as eligible for a spinal surgery procedure, are often viewed as having a mechanical problem suitable only for a mechanical solution such as a spinal device or fusion procedure, rather than a targeted biochemical therapy such as a TAT. Once the decision to proceed with surgery is made, the use of TATs is not usually even considered. To address some of these limitations, the inventor describes novel methods to identify patients who would benefit from TAT therapy, and to postpone, prevent, or improve the outcome of the the spinal surgery procedure.

A wide variety of inducers can cause inflammation in the body, including trauma, injury, disease, surgery, infection and cytokines. Such stimuli can induce the production of IC by a wide variety of cells, including cells of the immune system, cells of the central and peripheral nervous systems and cells from other tissues and organs (FIG. 1). Certain IC, such as TNF, IL-1, IL-6, IL-8, IL-12, IL-15, IL-17, IL-18, IL-23, IFN-γ, GM-CSF, and MCP-1, play key roles in the induction and maintenance of inflammation. A subset of cytokines called chemokines, such as IL-8 and MCP-1, function in concert with other IC during inflammation to recruit cells from the blood or cerebrospinal fluid to the site of injury. A wide variety of cell types comprise the inflammatory cell infiltrate (FIG. 1). Cells recruited to the site of injury, particularly monocytes, macrophages and dendritic cells, produce additional IC which collectively modulate cell maturation, proliferation, activation and angiogenesis. These IC act on both infiltrating cells and local tissue cells to produce and release inflammatory mediators (IM). Key IM include nitric oxide (NO), produced via activation of inducible NO synthase (iNOS), prostaglandinE2 (PGE2), an arachidonic acid metabolite resulting from the induction of the COX-2 enzyme, the matrix metalloproteinases (MMPs) MMP-1 (collagenase-1), MMP-2 (gelatinase A), MMP-3 (stromelysin), MMP-7 (matrilysin), MMP-9 (gelatinase B) and MMP-13 (collagenase-3), and the matrix-degrading aggrecanases ADAMTS4 and ADAMTS5 of the Adamalysin family of proteases. As illustrated in FIGS. 1, IC and IM act individually and in concert to cause inflammation and tissue damage, for example in irritation, inflammation, and injury of the spinal nerve root (NR). They also cause degradation of proteoglycans and extracellular matrix, as in matrix destruction in intervertebral disks and cartilage.

Role of ICs and IMs in Spinal Disorders

The causes of back and neck pain are diverse and complex and are inadequately served by available diagnostic and treatment options. Persistent and/or severe back and neck pain result from a variety of spinal disorders, including: spinal instability conditions such as spondylolysis, lytic spondylolisthesis, and degenerative spondylolisthesis (SLD); herniated disk (HD); spinal stenosis (SS); degenerative disk disease (DDD), such as that resulting from inflammatory and degenerative changes of the intervertebral disk, often called internal disk derangement, and sometimes manifesting as a clinical condition termed diskogenic pain; radicular pain conditions, often thought of as nerve compression disorders, such as sciatica; diseases resulting from inflammatory, degenerative, and other changes to the spinal vertebrae and their joints, such as facet joint deterioration; and complications of the spinal device or fusion procedures themselves.

Spinal disorders such as HD and SS cause mechanical compression of spinal NRs and nerves, initiating a biochemical cascade in which ICs and IMs play an essential role. The resulting NR injury, when significant, causes radiating pain along the distribution of the affected NR. This “radicular pain” is colloquially known as “sciatica” when occurring in the lower back and radiating into the buttock, thigh, or leg, in the distribution of the sciatic nerve. TNF and other ICs and IMs are increasingly implicated in controlling the pathophysiology of NR injury and resulting radiating pain. The potential efficacy of TNF-Is administered by intravenous (IV) or subcutaneous (SC) routes has also been tested in several preliminary human clinical trials in patients with HD and radiating pain, including one open label trial [1] and one blinded trial [2].

Conventional Treatment of Spinal Disorders

Therapy for spinal disorders typically progresses from conservative, non-invasive approaches to more aggressive, invasive approaches. Conservative treatment can include bed rest, the use of non-prescription anti-inflammatory agents and analgesics such as non-steroidal anti-inflammatory drugs (NSAIDs), traction, orthotic braces, and physical therapy. When relief provided by conservative therapies proves inadequate, treatment can progress to opioid analgesics and to more invasive, expensive epidural injections of steroids or local anesthetics (LAs). These moderately invasive measures performed by sub-specialists including anesthesiologists, radiologists and spine surgeons, may still be inadequate in the degree and/or durability of pain relief provided.

Therapy can then progress to more invasive procedures, such as invasive spinal procedures that do not require the implantation of a device or vertebral fusion including diskectomy for HD, and laminectomy for SS. Alternatively, or in addition, other invasive procedures that require the implantation of a device or vertebral fusion can be performed. The choice of which invasive procedure to use typically progresses from less aggressive, less invasive, more reversible procedures, such as nuclear replacement, annular repair, and dynamic stabilization device implantation procedures, to more aggressive, more invasive, less reversible procedures, such as disk replacement, facet joint replacement, or inter-vertebral fusion procedures. An anti-adhesive device may also be used with any of the procedures. The recommended invasive spinal procedure is based upon careful evaluation of the one or more spinal disorder(s) diagnosed in a given patient, and the specific invasive procedures available in the continuum of established and emerging invasive spinal therapies.

For many patients with severe or persistent spinal disorders, therapy progresses to a spinal device or fusion procedure. Due to the severity or persistence of their condition, patients meeting at least one SOE for a spinal device or fusion procedure are routinely offered surgical treatment as the standard of care, rather than drug therapies. Indeed, many have already failed to respond to conventional drug therapies such as NSAIDs or opioids. According to current practice, patients with moderate to severe spinal disorders are typically considered to be injured beyond the therapeutic abilities of non-invasive drug therapies, and thus to require surgical intervention to address the spinal disorder pathology or otherwise relieve the biomechanical imbalance in the spine. In contrast, patients considered as candidates for a drug therapy such as a TAT, e.g., a TNF-I, are typically those patients whose conditions are sufficiently non-severe to warrant recommendation for watchful waiting and non-invasive “conventional medical care,” rather than an invasive spinal procedure such as a spinal device or fusion procedure.

TAT Therapy of Patients Eligible for Spinal Device and Fusion Procedures

Thus, TAT therapy including TNF-I therapy is not currently practiced in patients eligible for spinal device or fusion procedures, or in patients who actually undergo such a procedure. The efficacy and suitability of these agents for this class of patients is surprising. Indeed, as described below, current practice and teaching poses specific barriers to use of TNF-Is in patients found eligible for a spinal device or fusion procedure, and additional barriers in patients who actually undergo such a procedure.

First, the currently marketed TNF-I compounds, Enbrel® (etanercept), Humira® (adalimumab), and Remicade® (infliximab), are protein therapeutics, either monoclonal antibodies or solube cytokine receptor fusion proteins. Enbrel®, Humira®, and Remicade® are approved for use by systemic routes of administration, either IV or SC. Such systemic agents are widely viewed as not crossing the blood brain barrier, and therefore of limited use for treating disorders of the spinal NR. The disk itself is also poorly vascularized and would not be expected to be substantially accessible to protein therapeutics administered by parenteral routes. There is little or no experience to guide the use of emerging TATs, most of which are protein therapeutics, by localized routes of administration such as epidural or intradiskal administration.

Second, treatment with the marketed TNF-Is has been linked with an increased risk of certain infections, a risk of significant potential concern to in spinal device and fusion procedures. This perceived potential for increased risk of infection presents a barrier to TNF-I use in patients eligible for or scheduled for a spinal device or fusion procedure. Chronic therapy with currently marketed TNF-Is is known to increase the risk of certain infections, particularly tuberculosis. Other rarer, sometimes serious infections have also been associated with use of TNF-Is. Therefore, TNF-Is use is contra-indicated in patients at high risk of infection, including patients with prior exposure to TB, with compromised immune systems, or with heightened risk of serious infection. Patients scheduled for or undergoing major surgery requiring exposure of deep musculoskeletal structures such as inter-vertebral disks are typically considered to be at heightened risk of serious infection. Many clinicians believe that TNF-I therapy may increase the risk of post-operative infection in surgery patients. While there is no convincing data to suggest that TNF-Is cause an increased risk of infection by the types of organisms found in post-operative infections in surgical patients, nevertheless, TNF-Is are typically not prescribed due to the concern regarding potential increased risk of infection. Thus, current perceptions of TNF-Is and practice in management of perceived infection risk mitigate against use of TNF-Is and other emerging TATs in patients found eligible for a spinal surgery procedure.

Similarly, once a determination is made that the patient will actually undergo the procedure, TATs are not prescribed. The spinal surgery procedure is viewed as likely to alleviate the mechanical disorder. The inventor has observed that even when a disk or lamina is removed, the procedure itself can further exacerbate the disorder, likely through activation of pathways that release ICs and IMs. Thus, patients undergoing a spinal surgery procedure are surprisingly, likely to benefit from an administration of a TAT such as a TNF-I, through improved outcome of the spinal surgery procedure.

In summary, many spinal disorder patients who fail to respond to conventional conservative (e.g., non-invasive) treatments will be found eligible for and will undergo a spinal device or fusion procedure. These invasive procedures are limited by inherent risks, high failure rates, and uncertain outcome. For patients eligible for a spinal device or fusion procedure, a need exists for improved non-surgical methods to provide rapid and substantial pain relief, in order to prevent or delay, if possible, the need for the spinal device or fusion procedure. In addition, for patients who do undergo a spinal device or fusion procedure, there is a need for effective, safe treatments to reduce the damage caused by the surgery procedure itself.

The invention provides a conceptually simple but surprising method of identifying patients as candidates for therapy with a TNF-I or other TAT. Contrary to current literature, teaching and practice, the disclosure provides that eligibility for a spinal device or fusion procedure, rather than identifying a subject as inappropriate for therapy with a TNF-I or other TAT, specifically identifies a patient as likely to benefit from TAT including TNF-I therapy. Through practice of the invention, many patients eligible to undergo a spinal device or fusion procedure will avoid the need for the procedure through practice of the invention. Moreover, for patients who do undergo such a procedure, therapy with a TNF-I or other TAT can improve the outcome and speed post-operative recovery.

### SUMMARY

The present disclosure is directed to identifying and treating subjects with spinal disorders who currently are typically not offered treatment with a TAT such as a TNF-I, but who could in many or most cases benefit from TAT treatment. The inventor has made the surprising discovery that patients suffering from moderate to severe disorders of the spine who have been determined to be eligible for a spinal device or fusion procedure are likely to benefit from TAT treatment to prevent, delay, or improve the outcome of the spinal device or fusion procedure.

The inventor has discovered that a subject or class of subjects can be reliably identified as highly likely to benefit therapeutically from TAT treatment if the subject or subjects meet(s) the eligibility criteria in one or more of the following SOEs for a spinal device or fusion procedure:

a) a determination of eligibility of the subject for the spinal device or fusion procedure by a healthcare service provider, as evidenced by:
i) a scheduling or request for scheduling by a healthcare service provider of the spinal device or fusion procedure for the subject;
ii) a communication by a healthcare service provider to the subject that the subject has been determined to be eligible for the spinal device or fusion procedure by the healthcare service provider;
iii) a provision or offering by a healthcare service provider to the subject of a consent form for the spinal device or fusion procedure, or an informed consent form for a clinical trial of the procedure;
iv) a receipt or execution by the subject of a consent form offered by a healthcare service provider for the spinal device or fusion procedure, or an informed consent form for a clinical trial of the procedure;
v) a notation by the healthcare service provider in a tangible medium such as the patient's written or electronic medical record that the patient is eligible for the spinal device or fusion procedure;

b) a determination of eligibility of the subject for the spinal device or fusion procedure by a qualified entity other than the subject's healthcare provider; and

c) the meeting by the subject of the eligibility criteria for a spinal device or fusion procedure in one or more CPG(s), or in a clinical trial of the spinal device or fusion procedure; see, e.g., TABLE 1 disclosed herein and Section IIIB below.

The methods provided herein may thus be useful in preventing or postponing the need for a spinal device or fusion procedure, or in improving the outcome of the procedure. While not being bound by theory, these benefits could be caused by preventing or reducing moderate to severe symptoms of the spinal disorder, such as by inhibiting or blocking the inflammatory cascades in which ICs and IMs play a role, and the consequent symptoms, including pain, such as persistent or radicular pain, and disability. The disclosure may be further useful in preventing or reducing injury to or irritation of the spinal NR, dorsal root ganglion, or peripheral nerve. Thus, the methods described herein may be useful in inducing remission from the troubling symptoms, such as persistent pain and/or radicular pain, which accompany the underlying pathologies of the spinal disorders described herein.

Accordingly, it is one object of the present invention to provide methods and materials for preventing, reducing, delaying, postponing, or eliminating the need for a spinal device or fusion procedure, or for improving the outcome of such procedures, by treating or reducing the symptoms and disability necessitating surgery, such as nerve injury and neuropathic pain. In one embodiment, the method of the present invention comprises identifying subjects likely to benefit therapeutically from treatment with a TAT, e.g., a TNF-I, who heretofore would not have been treated with the same. Such subjects have met at least one SOE for a spinal device or fusion procedure. For example, the present methods can include identifying as a subject likely to benefit from the therapies described herein (e.g., administration of a TAT such as a TNF-I), a subject with HD who is a candidate for a nuclear replacement device procedure according to the eligibility criteria in a CPG or a clinical trial of nuclear replacement.

Therapy consists of administration of a TAT as described herein. The TAT is administered either by a standard regimen and/or route, or by a novel regimen, for example, a novel regimen as described herein. For example, the TAT could be administered using an intradiskal/peridiskal regimen, as described herein. In other cases, a regimen could include administering (a) an induction regimen comprising a TAT (e.g., TNF inhibitor (TNF-I)); and (b) a maintenance regimen comprising a TAT (e.g., TNF-I). Any regimen can also involve temporary peri-operative interruption of the TAT, e.g., TNF-I, treatment course, in order to reduce the perceived risk of post-operative infection, with resumption regimen post-operatively. Provided herein also are teachings to guide selection of the proper timing and duration for peri-operative interruption of therapy at the discretion of the clinician responsible for managing the patient's therapy before, during, and/or after the spinal device or fusion procedure.

In an embodiment, described herein is a method of identifying a subject who could benefit therapeutically from administration of a direct TNF inhibitor (direct TNF-I), the method comprising determining that the subject meets at least one predetermined standard of eligibility (SOE) for a spinal device or fusion procedure, thereby identifying the subject as one who could benefit.

Also described herein is a method of identifying a subject who could benefit therapeutically from administration of an NFκB inhibitor (NFκB-I), the method comprising determining that the subject meets at least one predetermined SOE for a spinal device or fusion procedure, thereby identifying the subject as one who could benefit.

In an embodiment, these methods include a subject that is eligible for the following; for a disk nucleus replacement procedure; for an annular repair procedure; for a dynamic stabilization procedure; for an artificial disk procedure; for an interbody spine fusion; for a posterolateral fusion; for an interbody spine fusion using BMP-2; for kyphoplasty, vertebroplasty or vertebral restoration; for facet replacement; or for spinal procedure augmented by an anti-adhesive.

In these methods, the predetermined SOE is selected from the following: a) a determination of eligibility of the subject for the spinal device or fusion procedure by a healthcare service provider (as evidenced by: i) a scheduling or request for scheduling by a healthcare service provider of the spinal device or fusion procedure for the subject; ii) a communication by a healthcare service provider to the subject that the subject has been determined to be eligible for the spinal device or fusion procedure; iii) a provision or offering by a healthcare service provider to the subject of a consent form for the spinal device or fusion procedure; iv) a receipt or execution by the subject of a consent form for the spinal device or fusion procedure, said consent form provided by the subject's healthcare provider; or v) a notation by the healthcare service provider in a tangible medium that the patient is eligible for the spinal device or fusion procedure); b) a determination of eligibility of the subject for the spinal device or fusion procedure by a qualified entity other than the subject's healthcare provide; and c) the meeting by the subject of the eligibility criteria for a spinal device or fusion procedure in one or more CPG(s) or clinical trial(s). The above described method of identifying a subject who could benefit therapeutically from administration of a direct TNF inhibitor may further comprise recording the identification of the subject in a tangible medium; and administering a direct TNF-I to the subject.

In one aspect, the direct TNF-I is selected from the group consisting of an antibody or antibody fragment, a fusion protein, a peptide, a SMIP, a small molecule, an oligonucleotide (such as an siRNA), an oligosaccharide, a soluble cytokine receptor or fragment thereof, a soluble TNF receptor Type I or a functional fragment thereof, a polypeptide that binds to TNF, and a dominant negative TNF molecule. In a further aspect, the direct TNF-I is selected from the group consisting of: Humira® (adalimumab/D2E7); Remicade® (infliximab); Cimzia® (CDP-870); Humicade® (CDP-570); golimumab (CNTO 148); CytoFab (Protherics); AME-527; anti-TNF-Receptor 1 mAb or dAb; ABX-10131; polyclonal anti-TNF antibodies; anti-TNF polyclonal anti-serum; anti-TNF or anti-TNF-R SMIPs (Trubion); Enbrel® (etanercept); pegsunercept/PEGs TNF-R1, onercept; recombinant TNF binding protein (r-TBP-1); trimerized TNF antagonist; SSR-150106 (Sanofi-Synthelabo); ABX-0402 (Ablynx); nanobody therapeutics (Ablynx); trimerized TNF antagonist (Borean); humanized anti-TNF mAb (Biovation); Dom-0200 (Domantis); Genz-29155 (Genzyme); agarooligosaccharide (Takara Shuzo); HTDN-TNF (Xencor); and therapeutic human polyclonal anti-TNF and anti-TNF-R antibodies (THP).

In an embodiment, the method of identifying a subject who could benefit therapeutically from administration of an NFκB-I may further comprise administering an NFκB-I to the subject where the NFκB-I is selected from the group consisting of sulfasalazine, sulindac, clonidine, helenalin, wedelolactone, pyrollidinedithiocarbamate (PDTC), IKK-2 inhibitors, and IKK inhibitors.

In an embodiment, described herein is also a method for preventing or postponing a spinal device or fusion procedure in a subject, where the subject meets at least one predetermined SOE for a spinal device or fusion procedure. This method includes the following: a) optionally identifying the subject as a subject eligible for the spinal device or fusion procedure; b) administering to the subject a therapeutically effective amount of at least one direct TNF-I; and-c) optionally determining whether the subject's eligibility for the spinal device or fusion procedure has been prevented or postponed.

In an embodiment, described herein is also a method for preventing or postponing a spinal device or fusion procedure in a subject where the subject meets at least one predetermined SOE for a spinal device or fusion procedure. This method includes the following: a) optionally identifying the subject as a subject eligible for the spinal device or fusion procedure; b) administering to the subject a therapeutically effective amount of at least one NFκB-I; and c) optionally determining whether the subject's eligibility for the spinal device or fusion procedure has been prevented or postponed.

In one embodiment, the previous two methods include a subject eligible for; a disk nucleus replacement procedure; an annular repair procedure; a dynamic stabilization procedure; an artificial disk procedure; an interbody spine fusion; a posterolateral fusion; an interbody spine fusion using BMP-2; kyphoplasty, vertebroplasty or vertebral restoration; facet replacement; or a spinal procedure augmented by an anti-adhesive.

In these methods, the predetermined SOE is selected from: a) a determination of eligibility of the subject for the spinal device or fusion procedure by a healthcare service provider (as evidenced by: i) a scheduling or request for scheduling by a healthcare service provider of the spinal device or fusion procedure for the subject; ii) a communication by a healthcare service provider to the subject that the subject has been determined to be eligible for the spinal device or fusion procedure; iii) a provision or offering by a healthcare service provider to the subject of a consent form for the spinal device or fusion procedure; iv) a receipt or execution by the subject of a consent form for the spinal device or fusion procedure, said consent form provided by the subject's healthcare provider; or v) a notation by the healthcare service provider in a tangible medium that the patient is eligible for the spinal device or fusion procedure); b) a determination of eligibility of the subject for the spinal device or fusion procedure by a qualified entity other than the subject's healthcare provider; and c) the meeting by the subject of the eligibility criteria for a spinal device or fusion procedure in one or more CPG(s) or clinical trial(s). In one aspect, the subject is eligible for a disk nucleus replacement procedure based on the subject: 1) having been diagnosed with a) HD confirmed on MRI or b) mild to moderate DDD confirmed on MRI with a loss of disk height of less than 50 percent; and 2) having failed conservative treatment for a period of at least 6 weeks; having back or leg pain from L2-S1 with nerve root involvement or radicular neck pain; and not having facet arthropathy, SS, or spinal segment instability. In another aspect, these methods include a subject that is eligible for an annular repair procedure based on: 1) the subject having been diagnosed as having HD with MRI and/or CT confirmation and associated leg pain; and the subject having failed conservative treatment for a period of at least 6 weeks; or 2) the subject is undergoing nucleus replacement, and the treating spine interventionalist elects to perform conjoint annular repair. In a further aspect, the methods include a subject that is eligible for a dynamic stabilization procedure with a pedicle screw based device based on: 1) the subject having been diagnosed with one or more of the following: a) mild to moderate DDD; b)moderate to severe SS with back or leg pain from L2-S1; where either the DDD or stenosis is confirmed by MRI and/or CT; and c) pain originating from the disk, facet joints, and/or ligaments confirmed by physical/neurological examination; and 2) the failure of conservative treatment for a period of at least 6 months. In an alternative aspect, these methods include a subject that is eligible for a dynamic stabilization spinal procedure with an interspinous process spacer based on: A) the subject having been diagnosed with one of the following: 1) a) mild to moderate DDD or b) moderate to severe SS with back or leg pain from L2-S1, where either the DDD or stenosis is be confirmed by MRI and/or CT; and B) the subject is experiencing a) intermittent neurogenic claudication or b) low back pain improving with flexion, or c) radicular leg pain; and C) the failure of conservative treatment for a period of at least 6 months. In an aspect, these methods include a subject that is eligible for an artificial disk procedure based on: A) the subject having been diagnosed with moderate to severe DDD confirmed by MRI and/or CT and where the subject does not have severe facet arthropathy, gross spine instability, or vertebral body osteoporosis; where, for lumbar artificial disk procedures, the subject also experiences back or leg pain with provocative diskography and has failed at least 6 months of conservative therapy; and where, for cervical artificial disk procedures, the subject also experiences radiculopathy manifesting as neck or arm pain or a decrease in muscle strength and has failed conservative therapy for a minimum of 6 weeks. In one aspect, these methods include a subject that is eligible for an interbody spine fusion procedure based on: A) the subject having been diagnosed with DDD and one or more of the following: a) moderate to severe spinal instability; b) SS; and c) spondylolisthesis, with the diagnosis confirmed by either CT, and/or MRI, and/or x-ray; and B) the subject has back or neck pain that has failed conservative treatment for a minimum of 6 months. In a further alternative aspect, these methods include a subject that is eligible for a posterolateral fusion based on: A) the subject having been diagnosed with a) DDD with degenerative spondylolisthesis and/or b) SS, with the diagnosis confirmed by MRI and/or CT; and B) the subject has low back pain that has failed conservative treatment for a period of at least 6 months. In an aspect, these methods include a subject that is eligible for an interbody spine fusion procedure using BMP-2 based on: A) the subject having been diagnosed with DDD and one or more of the following: a) moderate to severe spinal instability; b) SS; and c) spondylolisthesis, with the diagnosis confirmed by either CT, and/or MRI, and/or x-ray; and B) the subject has back or neck pain that has failed conservative treatment for a minimum of 6 months. These methods also include a subject that is eligible for a kyphoplasty, vertebroplasty or vertebral restoration based on; A) the subject having been diagnosed with a vertebral compression fracture confirmed on x-ray, CT and/or MRI; and B) the subject experiences back pain correlated with the site of the vertebral compression fracture. Subjects may also be eligible for a facet replacement procedure (based on: A) the subject having been diagnosed with facet arthritis confirmed by CT and/or MRI and optionally with degenerative SS; and B) the subject experiences intermittent neurogenic claudication that worsens on walking or standing, coupled with radiological evidence of nerve root impingement by either osseous or non-osseous elements); a spinal procedure involving implantation of an anti-adhesive (based on: A) the subject being eligible for a spinal device or fusion procedure selected from the following: a) a disk nucleus replacement procedure; b) an annular repair device procedure; c) a dynamic stabilization procedure; d) an artificial disk procedure; e) an interbody spine fusion; f) a posterolateral fusion; g) an interbody spine fusion using BMP-2; h) a kyphoplasty, vertebroplasty or vertebral restoration; and i) facet replacement; or B) the subject being eligible for any spinal device or fusion procedure that does not involve the implantation of an implantable device or fusion of vertebrae). In an aspect, the spinal device or fusion procedure that does not involve the implantation or fusion is selected from diskectomy, laminectomy, percutaneous or endoscopic epidural adhesiolysis, radiofrequency neurotomy (RFN), and intradiskal electrothermal therapy (IDET).

In an embodiment, these methods include objectively or subjectively assessing the effect of a step involving administering to a subject a therapeutically effective amount of at least one direct TNF-I or an NFκB-I; on the subject, where the assessment comprises at least one of the following steps: a) determining a level or temporal duration of pain, impaired mobility, disability, or spinal nerve root irritation in the subject; b) determining an amount of TNF in the subject at a location of interest; c) fluoroscopically or radiologically observing the subject; d) determining whether the subject continues to meet the eligibility criteria in the predetermined SOE or CPG for the spinal device or fusion procedure; e) determining a measure of disability using the Oswetry Disability Index; f) determining a measure of functioning using the Short Form 36 Assay; g) optionally comparing the results of any one of steps a) to f) with the results of the same step performed prior to administration of at least one direct TNF-I or an NFκB-I. In an embodiment, the direct TNF-I and the NFκB-I may include 2 separate administrations of a direct TNF-I or an NFκB-I. In both cases, the method treats the subject so that the subject does not undergo a spinal device or fusion procedure in at least the first three months after the initial administration of the TNF-I, or treats the subject so that the subject does not undergo a spinal device or fusion procedure in at least the first three months after the initial administration of the NFκB-I.

In an embodiment, the direct TNF-I is selected from the group consisting of an antibody or antibody fragment, a fusion protein, a peptide, a SMIP, a small molecule, an oligonucleotide, an oligosaccharide (such as an siRNA), a soluble cytokine receptor or fragment thereof, a soluble TNF receptor Type I or a functional fragment thereof, a polypeptide that binds to TNF, and a dominant negative TNF molecule. Alternatively, the direct TNF-I is selected from the group consisting of: Humira® (adalimumab/D2E7); Remicade® (infliximab); Cimzia® (CDP-870); Humicade® (CDP-570); golimumab (CNTO 148); CytoFab (Protherics); AME-527; anti-TNF-Receptor 1 mAb or dAb; ABX-10131; polyclonal anti-TNF antibodies; anti-TNF polyclonal anti-serum; anti-TNF or anti-TNF-R SMIPs (Trubion); Enbrel® (etanercept); pegsunercept/PEGs TNF-R1, onercept; recombinant TNF binding protein (r-TBP-1); trimerized TNF antagonist; SSR-150106 (Sanofi-Synthelabo); ABX-0402 (Ablynx); nanobody therapeutics (Ablynx); trimerized TNF antagonist (Borean); humanized anti-TNF mAb (Biovation); Dom-0200 (Domantis); Genz-29155 (Genzyme); agarooligosaccharide (Takara Shuzo); HTDN-TNF (Xencor); and therapeutic human polyclonal anti-TNF and anti-TNF-R antibodies (THP). In one aspect, the NFκB-I is selected from the group consisting of sulfasalazine, sulindac, clonidine, helenalin, wedelolactone, pyrollidinedithiocarbamate (PDTC), IKK-2 inhibitors, and IKK inhibitors.

In a particular embodiment, the administration comprises: (a) an induction regimen comprising a direct TNF-I; and (b) a maintenance regimen comprising a direct TNF-I. Alternatively, the administration comprises: (a) an induction regimen comprising an NFκB-I; and (b) a maintenance regimen comprising an NFκB-I.

In one embodiment, the induction regimen is administered intrathecally, intradiskally, peridiskally, or epidurally, or combinations thereof. On the other hand, the maintenance regimen comprises systemic or parenteral administration, IV, perispinal, intramuscular, SC, or transdermal administration, administration by a pump, administration by implantation of a depot formulation or a hydrogel formulation.

In one embodiment, the induction regimen is completed prior to beginning administration of the maintenance regimen. Alternatively, the maintenance regimen may begin at or near the same time as the induction regimen.

In other embodiments, the induction regimen route of administration is selected from intra-operative, intrathecal, intradiskal, peridiskal, epidural (including periradicular and transforaminal), and the maintenance regimen route of administration is selected from perispinal, IV, SC, intramuscular, and transdermal. Additionally, the induction regimen may be administered locally to a site of the spine pathology of the subject (for example within 10 cm of the site of the spinal pathology), and the maintenance regimen is administered systemically or parenterally. In various embodiments, the induction regimen comprises a lower dose per administration to the subject than the maintenance regimen dose per administration.

In an alternative embodiment, the methods disclosed herein further comprise administering to the subject a therapeutically effective amount of a supplemental active ingredient (SAI), where the SAI is selected from the group consisting of a second TAT, a corticosteroid, ozone, an antirheumatic drug, an LA, a neuroprotective agent, a salicylic acid acetate, a hydromorphone, a non-steroidal anti-inflammatory drug, a cox-2 inhibitor, an antidepressant, an anticonvulsant, a calcium channel blocker, and an antibiotic.

It is conceieved that a direct TNF-I and an NFκB-I may be administered locally to a site of spine pathology of the subject. Such a route of administration may be selected from the group consisting of intra-operative, intrathecal, intradiskal, peridiskal, epidural (including periradicular and transforaminal), any combination of intradiskal, epidural, and peridural, perispinal, IV, intramuscular, SC, oral, intranasal, inhalation, and transdermal, or any combination thereof.

In an embodiment, described herein is a method for improving the outcome of a spinal device or fusion procedure in a subject, where the subject meets at least one predetermined SOE for a spinal device or fusion procedure. This method comprises the following: a) optionally identifying the subject as a subject eligible for the spinal device or fusion procedure; b) administering to the subject a therapeutically effective amount of at least one direct TNF-I; and c) performing the spinal device or fusion procedure, where the spinal device or fusion procedure is selected from a spinal device or fusion procedure that implants one or more of an annular repair or replacement device, a dynamic stabilization device, a kyphoplasty/vertebroplasty/vertebral restoration device, a facet replacement and fixation device, a dural repair device, or a spine fusion device.

In an embodiment, also described herein is a method for improving the outcome of a spinal device or fusion procedure in a subject, where the subject meets at least one predetermined SOE for a spinal device or fusion procedure. This method includes the following: a) optionally identifying the subject as a subject eligible for the spinal device or fusion procedure; b) administering to the subject a therapeutically effective amount of at least one NFκB-I; and c) performing the spinal device or fusion procedure, where the spinal device or fusion procedure is selected from a spinal device or fusion procedure that implants one or more of an annular repair or replacement device, a dynamic stabilization device, a kyphoplasty/vertebroplasty/vertebral restoration device, a facet replacement and fixation device, a dural repair device, or a spine fusion device. The previous two methods may include a patient that is eligible for: an annular repair procedure; a dynamic stabilization procedure; an interbody spine fusion; an interbody spine fusion using BMP-2; a posterolateral fusion; kyphoplasty, vertebroplasty or vertebral restoration; or facet replacement. In an aspect, the predetermined SOE is selected from; a) a determination of eligibility of the subject for the spinal device or fusion procedure by a healthcare service provider (as evidenced by: i) a scheduling or request for scheduling by a healthcare service provider of the spinal device or fusion procedure for the subject; ii) a communication by a healthcare service provider to the subject that the subject has been determined to be eligible for the spinal device or fusion procedure; iii) a provision or offering by a healthcare service provider to the subject of a consent form for the spinal device or fusion procedure; iv) a receipt or execution by the subject of a consent form for the spinal device or fusion procedure, said consent form provided by the subject's healthcare provider; or v) a notation by the healthcare service provider in a tangible medium that the patient is eligible for the spinal device or fusion procedure); b) a determination of eligibility of the subject for the spinal device or fusion procedure by a qualified entity other than the subject's healthcare provider; and c) the meeting by the subject of the eligibility criteria for a spinal device or fusion procedure in one or more CPG(s) or clinical trial(s). In an additional aspect, these methods include a subject, where the subject is eligible for an annular repair procedure based on: 1) the subject having been diagnosed as having HD with MRI and/or CT confirmation and associated leg pain; and the subject having failed conservative treatment for a period of at least 6 weeks; or 2) the subject is undergoing nucleus replacement, and the treating spine interventionalist elects to perform conjoint annular repair. These methods also include a subject eligible for; a dynamic stabilization procedure with a pedicle screw (based on: 1) the subject having been diagnosed with one or more of the following: a) mild to moderate DDD; b) moderate to severe SS with back or leg pain from L2-S1; where either the DDD or stenosis is confirmed by MRI and/or CT; and c) pain originating from the disk, facet joints, and/or ligaments confirmed by physical/neurological examination; and 2) the failure of conservative treatment for a period of at least 6 months); a dynamic stabilization spinal procedure with an interspinous process spacer (based on: A) the subject having been diagnosed with one of the following: 1) a) mild to moderate DDD or b) moderate to severe SS with back or leg pain from L2-S1, where either the DDD or stenosis is be confirmed by MRI and/or CT; and B) the subject is experiencing a) intermittent neurogenic claudication, or b) low back pain with improvement on flexion, or c) radicular leg pain; and C) the failure of conservative treatment for a period of at least 6 months); an interbody spine fusion procedure (based on: A) the subject having been diagnosed with DDD and one or more of the following: a) moderate to severe spinal instability; b) SS; and c) spondylolisthesis, with the diagnosis confirmed by either CT and/or MRI, or x-ray; and B) the subject has back or neck pain that has failed conservative treatment for a minimum of 6 months); a posterolateral fusion (based on: A) the subject having been diagnosed with a) DDD with degenerative spondylolisthesis and/or b) SS, with the diagnosis confirmed by MRI and/or CT; and B) the subject has low back pain that has failed conservative treatment for a period of at least 6 months); an interbody spine fusion procedure using BMP-2 (based on: A) the subject having been diagnosed with DDD and one or more of the following: a) moderate to severe spinal instability; b) SS; and c) spondylolisthesis, with the diagnosis confirmed by either CT and/or MRI, and/or x-ray; and B) the subject has back or neck pain that has failed conservative treatment for a minimum of 6 months); a kyphoplasty, vertebroplasty or vertebral restoration(based on A) the subject having been diagnosed with a vertebral compression fracture confirmed on x-ray, CT and/or MRI; and B) the subject experiences back pain correlated with the site of the vertebral compression fracture); and a facet replacement procedure (based on: A) the subject having been diagnosed with facet arthritis confirmed by CT and/or MRI and optionally with degenerative SS; and B) the subject experiences intermittent neurogenic claudication that worsens on walking or standing, coupled with radiological evidence of nerve root impingement by either osseous or non-osseous elements). In any of these embodiments, the direct TNF-I is selected from the group consisting of an antibody or antibody fragment, a fusion protein, a peptide, a SMIP, a small molecule, an oligonucleotide (such as an siRNA), an oligosaccharide, a soluble cytokine receptor or fragment thereof, a soluble TNF receptor Type I or a functional fragment thereof, a polypeptide that binds to TNF, and a dominant negative TNF molecule. The direct TNF-I may also be selected from the group consisting of: Humira® (adalimumab/D2E7); Remicade® (infliximab); Cimzia® (CDP-870); Humicade® (CDP-570); golimumab (CNTO 148); CytoFab (Protherics); AME-527; anti-TNF-Receptor 1 mAb or dAb; ABX-10131; polyclonal anti-TNF antibodies; anti-TNF polyclonal anti-serum; anti-TNF or anti-TNF-R SMIPs (Trubion); Enbrel® (etanercept); pegsunercept/PEGs TNF-R1, onercept; recombinant TNF binding protein (r-TBP-1); trimerized TNF antagonist; SSR-150106 (Sanofi-Synthelabo); ABX-0402 (Ablynx); nanobody therapeutics (Ablynx); trimerized TNF antagonist (Borean); humanized anti-TNF mAb (Biovation); Dom-0200 (Domantis); Genz-29155 (Genzyme); agarooligosaccharide (Takara Shuzo); HTDN-TNF (Xencor); and therapeutic human polyclonal anti-TNF and anti-TNF-R antibodies (THP). Alternatively, in any of these embodiments, the NFκB-I is selected from the group consisting of sulfasalazine, sulindac, clonidine, helenalin, wedelolactone, pyrollidinedithiocarbamate (PDTC), IKK-2 inhibitors, and IKK inhibitors. Also in any of these embodiments, the administration may comprise: (a) an induction regimen comprising a direct TNF-I; and (b) a maintenance regimen comprising a direct TNF-I. Administration may also comprise (a) an induction regimen comprising an NFκB-I; and (b) a maintenance regimen comprising an NFκB-I. As described above, the induction regimen may be administered intrathecally, intradiskally, peridiskally, or epidurally, or using combinations thereof and the maintenance regimen may comprise systemic or parenteral administration.

In an embodiment, a device implanted in the spinal device or fusion procedure is a source of a direct TNF-I and/or a source of an NFκB-I. In an embodiment, the implanted device is not a source of the SAI.

In embodiments where an SAI is included, the SAI is selected from the group consisting of a second TAT, a corticosteroid, ozone, an antirheumatic drug, an LA, a neuroprotective agent, a salicylic acid acetate, a hydromorphone, a non-steroidal anti-inflammatory drug, a cox-2 inhibitor, an antidepressant, an anticonvulsant, a calcium channel blocker, and an antibiotic, a second TAT, a corticosteroid, ozone, an antirheumatic drug, an LA, a neuroprotective agent, a salicylic acid acetate, a hydromorphone, a non-steroidal anti-inflammatory drug, a cox-2 inhibitor, an antidepressant, an anticonvulsant, a calcium channel blocker, and an antibiotic.

In an embodiment, herein is described a method for improving the outcome of a spinal device or fusion procedure in a subject, where the subject meets at least one predetermined SOE for a spinal device or fusion procedure, and where the spinal device or fusion procedure implants a device that is a source of a TAT, the method comprising: a) optionally identifying the subject as a subject eligible for the spinal device or fusion procedure; b) administering to the subject a therapeutically effective amount of at least one direct TNF-I that is in addition to the TAT derived from the implanted device; and c) performing the spinal device or fusion procedure.

In an alternative embodiment, herein is described a method for improving the outcome of a spinal device or fusion procedure in a subject, where the subject meets at least one predetermined SOE for a spinal device or fusion procedure, and where the spinal device or fusion procedure implants a device that is a source of a TAT, the method comprising: a) optionally identifying the subject as a subject eligible for the spinal device or fusion procedure; b) administering to the subject a therapeutically effective amount of at least one NFκB-I that is in addition to the TAT derived from the implanted device; and c) performing the spinal device or fusion procedure. In both of the previous embodiments, the subject may be eligible for; a disk nucleus replacement procedure; an annular repair procedure; a dynamic stabilization procedure; an artificial disk procedure; an interbody spine fusion; a posterolateral fusion; an interbody spine fusion using BMP-2; kyphoplasty, vertebroplasty or vertebral restoration; facet replacement; or spinal procedure involving implantation of an anti-adhesive device. In one aspect, the predetermined SOE is selected from: a) a determination of eligibility of the subject for the spinal device or fusion procedure by a healthcare service provider (as evidenced by: i) a scheduling or request for scheduling by a healthcare service provider of the spinal device or fusion procedure for the subject; ii) a communication by a healthcare service provider to the subject that the subject has been determined to be eligible for the spinal device or fusion procedure; iii) a provision or offering by a healthcare service provider to the subject of a consent form for the spinal device or fusion procedure; iv) a receipt or execution by the subject of a consent form for the spinal device or fusion procedure, said consent form provided by the subject's healthcare provider; or v) a notation by the healthcare service provider in a tangible medium that the patient is eligible for the spinal device or fusion procedure); b) a determination of eligibility of the subject for the spinal device or fusion procedure by a qualified entity other than the subject's healthcare provider; and c) the meeting by the subject of the eligibility criteria for a spinal device or fusion procedure in one or more CPG(s) or clinical trial(s). In an aspect, the direct TNF-I is selected from the group consisting of an antibody or antibody fragment, a fusion protein, a peptide, a SMIP, a small molecule, an oligonucleotide (such as an siRNA), an oligosaccharide, a soluble cytokine receptor or fragment thereof, a soluble TNF receptor Type I or a functional fragment thereof, a polypeptide that binds to TNF, and a dominant negative TNF molecule. In a further aspect, the direct TNF-I is selected from the group consisting of: Humira® (adalimumab/D2E7); Remicade® (infliximab); Cimzia® (CDP-870); Humicade® (CDP-570); golimumab (CNTO 148); CytoFab (Protherics); AME-527; anti-TNF-Receptor 1 mAb or dAb; ABX-10131; polyclonal anti-TNF antibodies; anti-TNF polyclonal anti-serum; anti-TNF or anti-TNF-R SMIPs (Trubion); Enbrel® (etanercept); pegsunercept/PEGs TNF-R1, onercept; recombinant TNF binding protein (r-TBP-1); trimerized TNF antagonist; SSR-150106 (Sanofi-Synthelabo); ABX-0402 (Ablynx); nanobody therapeutics (Ablynx); trimerized TNF antagonist (Borean); humanized anti-TNF mAb (Biovation); Dom-0200 (Domantis); Genz-29155 (Genzyme); agarooligosaccharide (Takara Shuzo); HTDN-TNF (Xencor); and therapeutic human polyclonal anti-TNF and anti-TNF-R antibodies (THP).

In another aspect, the NFκB-I is selected from the group consisting of sulfasalazine, sulindac, clonidine, helenalin, wedelolactone, pyrollidinedithiocarbamate (PDTC), IKK-2 inhibitors, and IKK inhibitors.

In the above embodiments, administration comprises: (a) an induction regimen comprising a direct TNF-I; and (b) a maintenance regimen comprising a direct TNF-I. Administration may also comprise: (a) an induction regimen comprising an NFκB-I; and (b) a maintenance regimen comprising an NFκB-I. In both cases, the induction regimen is administered intrathecally, intradiskally, peridiskally, or epidurally, or combinations thereof and the maintenance regimen comprises systemic or parenteral administration.

In an embodiment, herein described is a kit comprising an implantable spinal device selected from the group consisting of a nucleus replacement device, an annular repair device; a dynamic stabilization device, an artificial disk, a fusion device, a kyphoplasty or vertebroplasty device, and a facet replacement device, and a direct TNF-I. This direct TNF-I may be; a) contained within or on the implantable spinal device; b) contained in a vial; c) disposed within a syringe, catheter, pump, or delivery device adapted for epidural, intradiskal, or peridiskal administration, or any combination thereof, or d) disposed within a depot, hydrogel, or controlled-release formulation.

In an embodiment, herein described is a kit comprising an implantable spinal device selected from the group consisting of a nucleus replacement device, an annular repair device; a dynamic stabilization device, an artificial disk, a fusion device, a kyphoplasty or vertebroplasty device, and a facet replacement device, and an NFκB-I. This NFκB-I may be; a) contained within or on the implantable spinal device; b) contained in a vial; c) disposed within a syringe, catheter, pump, or delivery device adapted for epidural, intradiskal, or peridiskal administration, or any combination thereof, or d) disposed within a depot, hydrogel, or controlled-release formulation.

In an embodiment, herein described is a kit comprising an implantable spinal device and a TNF-I, where the TNF-I is contained within a vial or is disposed within a syringe, catheter, pump, or delivery device adapted for epidural, intradiskal, or peridiskal administration, or any combination thereof.

In an embodiment, herein described is a kit comprising an implantable spinal device and an NFκB-I, where the NFκB-I is contained within a vial or is disposed within a syringe, catheter, pump, or delivery device adapted for epidural, intradiskal, or peridiskal administration, or any combination thereof.

In each of the above described kit embodiments, the kit may further comprise an SAI. The implantable spinal devices of these kits may be selected from the group consisting of a nucleus replacement device, an annular repair device; a dynamic stabilization device, an artificial disk, a fusion device, a kyphoplasty or vertebroplasty device, and a facet replacement device, wherein the implantable spinal device comprises a TNF-I contained within or on the implantable spinal device.

In an embodiment, herein described is an implantable spinal device selected from the group consisting of a nucleus replacement device, an annular repair device; a dynamic stabilization device, an artificial disk, a fusion device, a kyphoplasty or vertebroplasty device, and a facet replacement device, wherein the implantable spinal device comprises an NFκB-I contained within or on the implantable spinal device.

Unless otherwise defined, all technical and scientific terms used herein have the meaning commonly understood by one of ordinary skill in the art to which this invention pertains. All publications, patent applications, patents, and other references mentioned herein are incorporated by reference in their entirety. In case of conflict, the present specification, including definitions, will control. The disclosed materials, methods, and examples are illustrative only and not intended to be limiting. Skilled artisans will appreciate that methods and materials similar or equivalent to those described herein can be used to practice the invention.

### BRIEF DESCRIPTION OF THE DRAWINGS

FIG. 1 demonstrates the ICs and IMs to which the TATs as described herein are directed.

FIG. 2 demonstrates the designated IC polypeptides TNF and IL-1 and the defined polypeptides of the TNF and IL-1 pathways.

FIG. 3 sets forth representative TNF-I doses for induction and maintenance regimens in pain patients using Humira® (adalimumab) or Enbrel® (etanercept).

FIG. 4 sets forth representative TNF-I doses for induction and maintenance regimens in pain patients using Remicade® (infliximab).

FIG. 5 sets forth representative TNF-I doses for induction and maintenance regimens in pain patients using Cimzia (certolizumab pegol, CDP870).

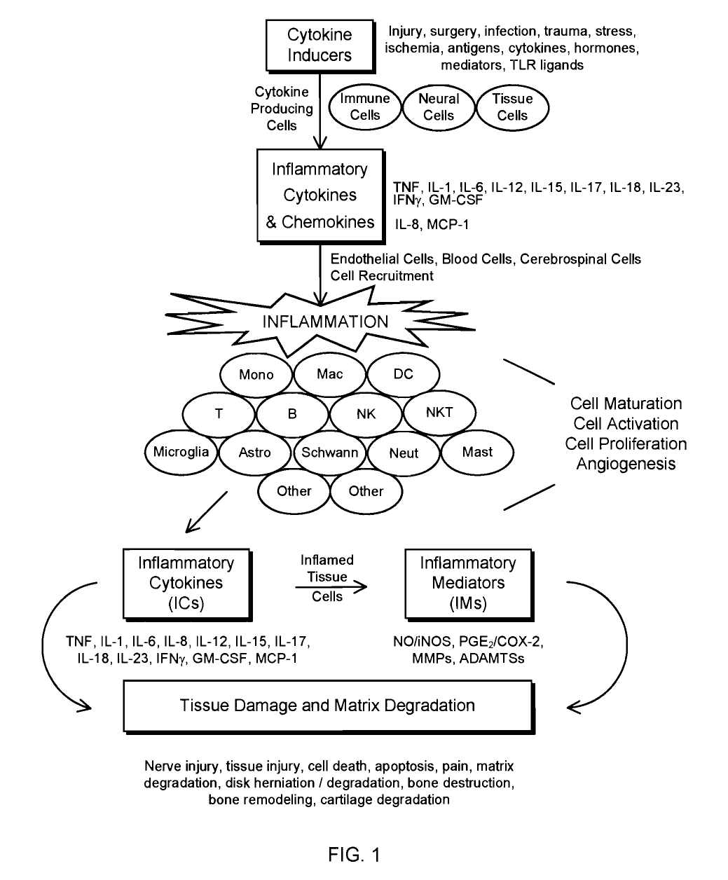

### DETAILED DESCRIPTION

### DEFINITIONS

Typically, and unless otherwise indicated, the term “spinal device and fusion procedure” refers to a spinal procedure, often surgical, that requires invasive manipulation of spinal tissues with implantation of an implantable device or fusion of two or more of the intervertebral vertebrae. Examples of such spinal device or fusion procedures include nucleus replacement; annular repair; dynamic stabilization (including implantation of pedicle-screw based devices or interspinous spacer devices); disk arthroplasty (implantation of an artificial disk); fusion of the vertebrae (sometimes augmented by use of a growth factor such as BMP-2); posterolateral spinal fusion procedures; kyphoplasty/vertebroplasty; facet replacement procedures; and any spinal procedures involving the implantation of an anti-adhesion barrier or gel. Repeat or revision embodiments of such spinal device or fusion procedures are also included within the definition.

As used herein, the terms “tumor necrosis factor,” “tumor necrosis factor-alpha,” “TNF,” and “TNF-α” are used interchangeably to refer to a naturally occurring cytokine, which plays a key role in the inflammatory response, in the immune response and in the response to infection. The term “human TNF” (abbreviated as huTNF or hTNF), as used herein, is intended to refer to a human cytokine that exists as a 17 kiloDalton (kD) secreted form and a 26 kD membrane associated form, the biologically active forms of which are composed of trimers of noncovalently bound 17 kD or 26 kD molecules respectively.

As used herein, the term “inflammatory cytokine” is used interchangeably with “IC” and refers to one of the following designated polypeptides: TNF, IL-1, IL-6, IL-8, IL-12, IL-15, IL-17, IL-18, IL-23, IFN-γ, GM-CSF, MCP-1, IL-8 and MCP-1.

As used herein, the term “inflammatory mediator” is used interchangeably with “IM” and refers to one of the following: MMP-1 (collagenase-1), MMP-2 (Gelatinase A), MMP-3 (stromelysin), MMP-7 (Matrilysin), MMP-9 (gelatinase), MMP-13 (collagenase-3), ADAMTS4, ADAMTS5, iNOS, NO, COX-2, and PGE2.

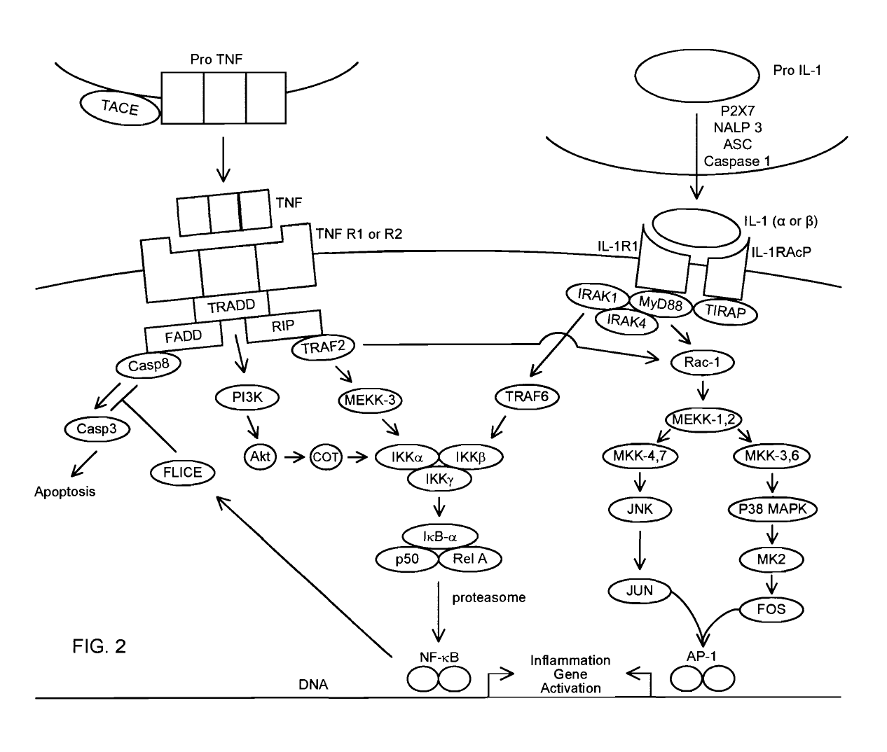

As used herein, the terms “inflammatory cytokine inhibitor” and “IC-I” are used interchangeably and refer to any molecule that blocks, suppresses or reduces gene expression, protein production and processing, protein release, and/or biological activity of: a) one of the following designated polypeptides: TNF, IL-1, IL-6, IL-12, IL-15, IL-17, IL-18, IL-23, IFNg, GM-CSF, and IL-8 (CXCR8) and MCP-I (CCL2), or the designated polypeptide's biological receptor, coreceptor, or coligand, as described above, or b) one of the defined polypeptides within the designated polypeptide's pathway, as described above and described further below. See also, e.g., FIG. 2 for a depiction of the defined polypeptides in the TNF and IL-1 pathways.

An IC-I can be a “direct IC-I,” meaning a molecule (e.g., an antibody (Ab) or fusion polypeptide) that binds directly to and inhibits the biological activity of a designated polypeptide, its receptor, coreceptor, or coligand, or is a molecule (e.g., a nucleic acid such as an siRNA or antisense molecule) that binds directly to a nucleic acid molecule encoding the designated polypeptide or its receptor, coreceptor, or coligand and inhibits or reduces the expression of the designated polypeptide or its receptor, coreceptor, or coligand.

As used herein, the terms “inflammatory mediator inhibitor” and “IM-I” are used interchangeably and refer to any molecule that blocks, suppresses or reduces gene expression, protein production and processing, protein release, and/or biological activity of one of the following IMs: MMP-1 (collagenase-1), MMP-2 (Gelatinase A), MMP-3 (stromelysin), MMP-7 (Matrilysin), MMP-9 (gelatinase), MMP-13 (collagenase-3), ADAMTS4, ADAMTS5, iNOS, NO, COX-2, and PGE2. An IM-I can be a “direct IM-I,” meaning a molecule (e.g., an Ab or fusion polypeptide) that binds directly to and inhibits the biological activity of MMP-I (collagenase-1), MMP-2 (Gelatinase A), MMP-3 (stromelysin), MMP-7 (Matrilysin), MMP-9 (gelatinase), MMP-13 (collagenase-3), ADAMTS4, ADAMTS5, iNOS, NO, COX-2, or PGE2, or meaning a molecule (e.g., a nucleic acid such as an siRNA or antisense molecule) that binds directly to a nucleic acid molecule encoding any of the foregoing IMs, inhibiting or reducing its expression.

Unless otherwise indicated, “small molecule,” and “small molecule inhibitor” are used interchangeably to refer to a molecule of low relative molecular mass that blocks, suppresses or reduces biological activity of a designated polypeptide. The term “low relative molecular mass” has art-recognized meaning, and refers to a molecule having a relative small number of atoms, typically less than 100 atoms (as compared to a protein, “biologic” or “macromolecule”). A small molecule can have a molecular weight of about 100 to 5000 daltons, e.g., about 500 to about 2000 daltons, or about 500 to about 1200 daltons.

As used herein, the terms “non-operative treatment” and “conventional non-invasive treatments” and “conservative care” are used interchangeably and mean one or more of watchful waiting by a healthcare provider, exercise, bed rest or reduced activity, physical therapy, administration of an NSAID, administration of a steroid, the use of an orthotic brace, and administration of oral analgesics including opioid analgesics.

As used herein, the term “peri-operative” means relating to, occurring in, or being the period around the time (e.g., before, during, and/or after) of a surgical operation.

“Interspinous route” refers to parenteral injection through the skin in the midline, in the interspace between two spinous processes, or via a paramedian approach, to deliver the therapeutic agent(s) in anatomic proximity to the spine.

“Intrathecal” means injection into the spinal canal (intrathecal space surrounding the spinal cord and intradural).

“Epidural” means in the space between the pia and dura mater, in which the nerve roots typically are found. “Periradicular” and “transforaminal” refer to specific types of epidural administration. “Periradicular” means within the epidural space, specifically in the region of the radicles (nerve roots). “Transforaminal” means through the vertebral foramen and within the epidural space, specifically in the region of the radicles. The terms “radicle” and “nerve root” are used interchangeably.

“Intradiskal” means penetration of the outer wall and into the nucleus pulposus of a disk and/or into the annulus fibrosus of a disk.

“Peridiskal” means adjacent to an outer wall of the annulus fibrosus; outside but closely adjacent to an outer wall of the annulus fibrosus; and/or outside but closely adjacent to an endplate of an adjacent vertebral body.

“Perispinal” means in the paraspinal muscles.

“Intradiskal/epidural” means a combination of intradiskal, as defined above, and epidural, as defined above. For example, an “intradiskal/epidural” administration of a TAT could include administration of the TAT into the nucleus pulposus of a disk and administration of the TAT into the epidural space, e.g., using a needle adapted for intradiskal administration to administer the TAT intradiskally, followed by injection epidurally, either with the same or a different needle.

“Intradiskal/peridiskal” means a combination of intradiskal, as defined above, and peridiskal, as defined above. For example, an “intradiskal/peridiskal” administration of a TAT could include administration of the TAT into the nucleus pulposus of a disk and administration of the TAT into the peridiskal space adjacent to an outer wall of the annulus fibrosus, e.g., using a needle adapted for intradiskal administration to administer the TAT intradiskally, followed by injection peridiskally, either with the same or a different needle.

“Intradiskal/peridiskal/epidural” means a combination of intradiskal, peridiskal, and epidural, as defined above. For example, an “intradiskal/peridiskal/epidural” administration of a TAT could include administration of the TAT into the nucleus pulposus of a disk and administration of the TAT into the peridiskal space adjacent to an outer wall of the annulus fibrosus, and further administration of a TAT into the epidural space.

As used herein, an “induction regimen” has the following properties: it is administered by: 1) a more invasive route of administration than a maintenance regimen or more local site of administration than a maintenance regimen; and 2) a lower dose per administration than the dose per administration used in the maintenance regimen administered to the same subject, concurrent with or following the induction regimen.

As used herein, “treatment” means any manner in which one or more of the symptoms of a disease or disorder are ameliorated or otherwise beneficially altered. As used herein, amelioration of the symptoms of a particular disorder refers to any lessening, whether permanent or temporary, lasting or transient that can be attributed to or associated with treatment by the methods of the present invention.

A “therapeutically effective amount” is an amount sufficient to affect a beneficial or desired clinical result, such as prevention or treatment of injury and/or pain; the prevention, delaying, postponement, reduction, or elimination of the need for an invasive surgical procedure; or an improvement in the outcome of a subject that undergoes an invasive procedure.

As used herein, “delaying” or “postponing” are used interchangeably and mean to defer, hinder, slow, retard, and/or stabilize a subject's need for or eligibility for an invasive surgical procedure. This delay can be of varying lengths of time, depending on the history of the disease and/or individuals being treated. As is evident to one skilled in the art, a sufficient or significant delay can, in effect, encompass prevention, in that the individual does not need the procedure. A method that “delays” or “postpones” exhibition of the need for or the eligibility for the invasive procedure is a method that reduces probability of the need for or the eligibility for the procedure in a given time frame, when compared to not using the method. Such comparisons can be based on clinical studies, using a group of subjects sharing similar disease characteristics.

As used herein, a method for “improving the outcome” of an invasive procedure refers to a method that, for example, reduces severity or intensity of pain, symptoms, or disability, results in alleviation of one or more symptoms associated with the disease or disorder, reduces resting pain and/or mechanically-induced pain, shortens the duration of pain, symptoms, or disability, and/or reduces pain sensitivity or sensation, in a given time frame after the procedure when compared to the outcome observed when not using the recited method. Other examples of improved outcome are set forth further herein. Such comparisons can be based on clinical studies, using a group of subjects sharing similar disease characteristics.

As used herein, and unless otherwise indicated, the terms “patient,” “subject,” and “individual” are used interchangeably to refer to a vertebrate, and particularly a mammal including, without limitation, humans, farm animals, sport animals, pets, primates, horses, dogs, cats, mice and rats.

As used herein, the term “invasive,” when in the context of administration of a TAT, refers to the degree to which a particular administration regimen or mode of administration involves penetration of the delivery vehicle into the body, organ, or internal structures. A more invasive mode of administration refers to greater penetration into the body, organ, or internal structures than a less invasive mode. For example, a more invasive mode of administration can be evidenced through use of a longer needle, e.g., to penetrate further into the body, organ, or internal structures. Thus, intramuscular administration is more invasive than subcutaneous (SC) as the administration is deeper into the body. A more invasive mode of administration can be evidenced by the use of a catheter to administer into an internal organ, artery, or vein. A more invasive mode of administration can be evidenced by the requirement for local anesthesia during the procedure, e.g., to minimize accompanying pain directly due to the invasive procedure. A more invasive mode can be evidenced by a requirement for image guidance (e.g., ultrasound or radiographic imagery to guide the procedure) for the procedure (e.g., flouroscopy for epidural or intradiskal administration). In some cases, a more invasive mode can involve greater risk, discomfort, or inconvenience to subject.

The following modes of administration are listed in order of invasiveness from highest to lowest: intra-operative, meaning into a surgical wound, to directly influence inflammation at the site of the surgical wound (e.g. into the wound in the region of the NR or disk); intradiskal; peridiskal and intrathecal administration; epidural administration, including periradicular and transforaminal; IV; perispinal and intramuscular; SC; and all other non-invasive modes of administration, including oral, intranasal, buccal, (including intrapulmonary and intrabronchial), and transdermal.

The term “pain” includes nociception and the sensation of pain, both of which can be assessed objectively and subjectively, using pain scores and other methods well-known in the art. Pain, as used herein, includes allodynia (i.e., increased response to a normally non-noxious stimulus) and hyperalgesia (i.e., increased response to a normally noxious or unpleasant stimulus), which can in turn, be thermal or mechanical (tactile) in nature. In some embodiments, pain is characterized by thermal sensitivity, mechanical sensitivity and/or resting pain. In other embodiments, pain comprises mechanically-induced pain or resting pain. In still other embodiments, the pain comprises resting pain. The pain can be primary or secondary pain, as is well-known in the art. Exemplary types of pain preventable or treatable by the methods of the present invention include, without limitation, back pain in the lumbar regions (low back pain) or cervical region (neck pain), leg pain, sciatic pain, radicular pain (experienced in the lower back and leg from lumber pathology, or in the neck and arm from cervical pathology), and neuropathic pain of the arm, neck, back, lower back, leg, and related pain distributions resulting from disk and spine pathology.

As used herein, “neuropathic pain” means pain arising from injury to the NR, dorsal root ganglion, or peripheral nerve.

As used herein, “post-surgical pain” and “surgery-induced pain” are used interchangeably, and refer to pain arising in the recovery period of days or weeks following a spine surgical procedure. Specific examples of such pain that occur with increased frequency after spinal device or fusion include, without limitation, leg pain, back pain, neck pain, and/or arm pain. “Resting pain” refers to pain occurring even while the individual is at rest as opposed to, for example, pain occurring when the individual moves or is subjected to other mechanical stimuli. “Mechanically-induced pain” (interchangeably termed mechanosensory pain) refers to pain induced by a mechanical stimulus, such as the application of weight to a surface, tactile stimulus, and stimulation caused or associated with movement (including coughing, shifting of weight, etc.).

I. Spinal Disorders

Patients with spinal disorders eligible for a spinal device or fusion procedure can be treated using the methods described herein, to prevent the need for the procedure and/or to improve its outcome. Examples of the most frequent injuries or conditions rendering a patient eligible for a spinal device or fusion procedure include: spinal instability conditions such as spondylolysis, lytic spondylolisthesis, and degenerative spondylolisthesis (SLD), HD; SS; DDD, such as that resulting from inflammatory and degenerative changes of the intervertebral disk, often called internal disk derangement, and sometimes manifesting as a clinical condition termed diskogenic pain; radicular pain conditions, often thought of as nerve compression disorders, such as sciatica; diseases resulting from inflammatory, degenerative, and other changes to the spinal vertebrae and their joints, such as facet joint deterioration; and complications of the spinal device or fusion procedures themselves.

1.) Spinal Instability/Spondylolysis/Lytic Spondylisthesis/Degenerative Spondylolisthesis (SLD) [3]

Spondylolisthesis occurs when one vertebra moves anteriorly in relation to an adjacent vertebra, usually in the lumbar spine (particularly L4-L5). This translation negatively affects the biomechanical function of that motion segment, and can lead to accelerated degeneration of the intervertebral disk. Degenerative spondylolisthesis usually occurs after age 50, often causing or exacerbating SS (a narrowing of the spinal canal). Subjects are diagnosed with spondylolisthesis using radiologic imaging techniques (x-ray or CT) to confirm anterior translation of a vertebra in the correct location and degree to correlate with the clinical symptoms of pain. The pain may be localized in the lumbosacral region, often radiates down one or both legs, and can affect the peroneal nerves. Stiffness of the back and tight hamstring muscles are strong diagnostic predictors of spondylolisthesis-associated pain, and flexion relieving this pain indicates that the spondylolisthesis has caused SS at the affected level. Selection of appropriate surgical options for treatment is based on clinical presentation, confirmed by imaging, and consideration of the patient's condition, related disorders, and the ability of a given intervention and device to address on or more related disorders.

2.) Herniated Disk

Severe or persistent back pain and radicular pain are frequently associated with herniation of the intervertebral disk. The intervertebral disk is composed of a fibrous outer ring, the annulus fibrosus and a proteoglycan-rich gel-like core, the nucleus pulposus. The annulus constrains the nucleus. HD is due to tears, fissures, or delamination of the annulus fibrosis. Disruption of the annulus allows a portion of the disk, including the nucleus and possibly components of the annulus, to protrude from the normal disk space. This disk protrusion comes in contact with and compresses the spinal NR, causing severe pain. Depending on the cause and nature of the disk protrusion, the protruding disk may be referred to as, for example, extruded, protruded, slipped, herniated, or prolapsed.

Though more common in the lower back (lumbar and sacral spine), herniation can occur at any level in the spine, including in the neck (cervical spine), which results in neck and/or arm pain (cervical radicular pain). In patients with herniation in the lower back, persistent pain can originate in the back and often extends into the leg (lumbar radicular pain, or “sciatica”). In patients with herniation in the neck, the persistent pain can originate in the neck and often extends into the arm.

Patients are diagnosed with HD by the history of persistent pain for a period of weeks, accompanied by characteristic abnormalities in the physical and neurological examination, and confirmation by appropriate imaging studies such as MRI. Abnormalities on pysical examination include limited mobility or range of motion, positive signs of NR irritation, such as reduced ability to raise the legs (positive straight leg raise test). Abnormalities in the neurological examination include reduced strength and sensation of particular parts of the body related to specific affected spinal NR. The diagnosis is typically confirmed by an MRI or CT showing an HD at the right location to explain the symptoms and signs found by history, physical and neurological exam.

3.) Spinal Stenosis

SS is a condition that involves the narrowing of the spinal canal and neural foramina, due to degenerative changes in the intervertebral disks, intervertebral joints (facet joints) and the ligamentum flavum. These degenerative changes leads to hypertrophy of the ligament flavum and facet joints, resulting in a gradual narrowing of the lumbar (back) or cervical (neck) spinal canal, causing compression of the spinal cord and NR. This narrowing puts pressure on the spinal cord and nerves leading to intermittent neurogenic claudication. Symptoms include pain and/or numbness in the neck, back, buttocks, legs, thighs or calves that is worse with walking, standing and/or exercise; back pain that radiates to the legs; weakness of the legs; and difficulty or imbalance when walking.

SS is diagnosed by clinical evaluation with confirmation by imaging studies. Clinical evaluation includes history, and assessment of the type and severity of the pain, examination of the reflexes of the lower legs to reveal asymmetry, and neurological examination to assess for the presence of weakness and decreased sensation in the legs. MRI and/or CT imaging is used to confirm stenosis at the appropriate vertebral level to explain the clinical symptoms of pain.

4.) Degenerative Disk Disease with Internal Disk Derangement and Positive Diskography (Diskogenic Pain)

Degenerative disk disease (DDD) is characterized by structural deficits in the disk that are directly related to aging and other pathological processes, and may be exacerbated by trauma. Moderate to severe DDD is prevalent worldwide. Patients with early signs of DDD on MRI and a characteristic history are often diagnosed with diskogenic pain. Fusion surgery has demonstrated a 95% or greater success in achieving vertebral fusion, but only about a 70% success in treating diskogenic pain, probably reflecting the variable causes of pain, and the limitations of distinguishing specific causes using currently available diagnostic approaches. While inter-vertebral fusion surgery, now frequently augmented by the use of BMPs and other growth factors, has been the gold for moderate to severe DDD, the current trend is toward less invasive, mobility-preserving approaches. These procedures involve devices such as nuclear replacement, annular repair, dynamic stabilization, artificial disks, facet joints, and so forth.

Diagnosis of diskogenic pain typically involves the combination of a characteristic history of pain with back flexion or with standing and improving with short walks, physical exam findings including limited muscle tenderness, and MRI studies showing characteristic findings such as loss of disk height or darkened color reflecting disk dehydration. The diagnosis is confirmed by the use of lumbar diskography, a provocative invasive diagnostic procedure. In order to ascertain whether the pain is due to derangement of the disk, fluid is injected into the disk along with a contrast agent.

5.) Radicular Pain/Radiculopathy (Sciatica)

Sciatica is characterized by radiating pain in an area of the leg typically served by NR root in the lumbar or sacral spine, often accompanied by sensory and motor deficiencies in the same area. The most common cause of sciatica is HD. Sciatica is characterized by pain radiating from the lower (lumbar) spine to the buttock and down the back of the leg. Lumbar SS or DDD can also cause compression of the spinal NR resulting in sciatica. Pain in the neck due to cervical disk disease can also radiate into the arm, causing cervical radicular pain.

Diagnosis of sciatica is based on MRI scan confirming an HD, chronic leg pain with pain in the lower extremity equal to or greater than the back pain, and often numbness or muscle weakness in the affected leg or foot.

6.) Facet Joint Disease/Syndrome

The facet joints provide the cartilaginous articular bearing surfaces of the spine, and are surrounded by an innervated capsule filled with synovial fluid. Hence, these joints are subject to degenerative arthritic changes much like those observed with hip or knee arthritis, and the capsular innervation can signal pain when degeneration occurs. In facet joint disease, these articular bearing surfaces become worn. Cartilage thinning leads to a reaction of the underlying bone causing osteophytes (bony protrusions) to form, resulting in overall enlargement of the joints. Osteophytes can impinge on the NR causing pain, and can also limit mobility of the associated motion segment.

Facet joint disease is diagnosed clinical evaluation including the presence of characteristic pain upon lateral flexion suggestive of a foraminal NR irritation, and pain that is greatest in the morning upon awakening and initial ambulation. MRI and/or CT imaging is used to confirm the presence and degree of facet degeneration.

7.) Adhesions/Scarring/Fibrosis (Post-Laminectomy, Peridural/Epidural Fibrosis, Nerve Entrapment)

Epidural fibrosis is the formation of fibrotic scar tissue near the NR following a spinal device or fusion procedure. The resulting NR irritation, inflammation and entrapment can cause recurring leg and back pain. Incidence of symptomatic epidural fibrosis is estimated at 10% of spinal device or fusion procedures, and it is often considered one of the complications listed under the general category of post laminectomy syndrome or FBSS. Binding of lumbar nerve root fibers by fibrous adhesions is believed to be the mechanism by which epidural fibrosis causes recurring pain. Diagnosis of epidural fibrosis is made by a history, physical and neurological exam suggestive of NR irritation, possibly confirmed by a positive finding of scar tissue by MRI. Symptoms associated with epidural fibrosis appear at 6 to 12 weeks after surgery, preceded by an initial period of pain relief that leads the subject to believe that the spine surgical procedure was a success. Following initial recovery, pain recurs. A positive straight leg-raise test is suggestive of NR entrapment. Current treatment options for epidural fibrosis are limited.

8.) Complications of Spinal Device or Fusion Procedures

Spinal device or fusion procedures can result in unique complications including, for example, epidural, peridural or other fibrosis, adhesions or scarring with or without NR entrapment; adjacent level disease in which the disks or joints adjacent to a joint that has been repaired begin to worsen following a spinal procedure; distraction injury during disk replacement, or ectopic calcification following disk arthroplasty; BMP-induced radiculitis following fusion with BMP augmentation; and failed back surgery syndrome (FBSS), in which a spinal procedure is followed by persistent or worsening symptoms. Many complications of spinal device or fusion procedures may be prevented, reduced or treated with the use of TATs as practiced in the invention.

Adjacent level disease is a condition that is seen months or years after spinal fusion surgery. In adjacent level disease, the fusion of two or more vertebrae into one motion segment, increases the loading on the disks adjacent to the fused vertebrae. This increase in loading can cause or accelerate DDD in these adjacent motion segments. Recently developed devices such as artificial disks and dynamic stabilization devices are designed to avoid fusion surgery, thereby preserving mobility and possibly reducing incidence of adjacent level disease.

Distraction injury and ectopic calcification are two complications of disk replacement that can be prevented or treated using TATs as practiced in the invention. During disk replacement surgery, as well as fusion surgery and some forms of dynamic stabilization implantation, the vertebral bones must be separated to allow removal of the degenerated disk, and insertion of the artificial disk device. This separation or “distraction” of the vertebrae can result in injuries to the manipulated spinal structures, including for example annular tears, due to stretching and manipulation of an annulus that is often itself stiffened or degenerated, or injury to the interspinous ligaments or vertebral body endplates. These injuries (“distraction injuries”) occur in about 25% of disk arthroplasty procedures, leading to irritation and inflammation of the nearby NR and intense post-operative pain in the back, often radiating to the leg or arm innervated by the affected NR. Treatment of post-surgical pain resulting from distraction injury typically involves the use of anti-inflammatory agents such as oral or locally administered epidural steroids. However, significant need remains for better methods to prevent and/or treat the pain resulting from distraction injury. The inventor has discovered and confirmed that administration of TATs as practiced in the invention can prevent, reduce, or treat the symptoms of distraction injury following disk arthroplasty or other spinal device or fusion procedures.

Likewise, following disk replacement surgery, implanted devices are prone to abnormal accumulation of calcium in or around the device, termed ectopic calcification. The abnormal deposition of calcium crystals leads to an accumulation of macroscopic hydroxyapatite deposits, which can cause the implanted device to fuse, freeze or otherwise malfunction. Although therapies exist for treating ectopic calcification associated with systemic mineral imbalance, there is no effective means for preventing local ectopic calcification due to injury and inflammation (dystrophic calcification), which is the leading cause of device failures. While anti-inflammatories could in theory reduce ectopic calcification, they are not used in current practice due to concern that inflammation is required to obtain appropriate healing of the bone and related tissues, and required firm seating of the implanted artificial disk. Indeed, anti-inflammatories such as cyclo-oxygenase inhibitors, for example Celebrex or Vioxx, are usually discontinued prior to disk arthroplasty due to this concern about inhibition of bone healing. The inventor has recognized that administration of TATs as practiced in the invention can prevent or reduce ectopic calcification following disk arthroplasty, while allowing proper bone healing to occur.

One particular complication of intervertebral fusion surgery with use of BMPs is been termed BMP-induced radiculitis, and has been identified by the inventor as an inflammatory complication directly related to the use of BMPs. Tissue swelling, even resulting in airway compromise, following use of BMPs in cervical fusions is well documented, and has resulted in caution on the part of many surgeons in using BMPs to augment cervical fusion procedures. While tissue swelling may be observed in lumbar procedures, because it tends to be self-limiting, subside in the days or weeks following surgery, and not cause urgent or emergent complications, this swelling is viewed as acceptable in lumbar fusion procedures. The inventor has performed survey research involving systematic interviews with many different spine surgeons. In analyzing the results of these interviews, the inventor noted that the majority of surgeons report that they observe frequent cases of patients with onset of new, intense radicular pain following lumbar fusion surgery in which BMPs are used. The inventor terms this condition BMP-induced radiculitis, which he believes is a consequence of NR inflammation induced by the use of BMPs in the fusion procedure. Surgeons and the pain specialists whom they consult treat this condition with steroids, with limited efficacy. BMP-induced radiculitis could be prevented or treated by appropriate peri-operative TAT administration as practiced in the invention.

9.) FBSS with Device Revision, Removal or Replacement

Spinal surgery can fail for a complex variety of interrelated reasons including: the accuracy of the initial diagnosis and the choice of the appropriate spinal device or fusion procedure; surgical technique; scarring that may or may not be preventable; and confounding psychosocial subject related variables, including possible financial gain from work related injuries. Improper diagnosis of the underlying cause of the symptoms will lead to failure of the procedure to resolve the patient's symptoms. Improper technique as well as the inherently challenging technical nature of the procedures can result in failure due to, for example, loss of fixation of implants. Epidural fibrosis and scarring naturally occur following epidural or spinal procedures, and are not completely preventable using current standard of care. Finally, the subject's psychosocial characteristics may provide a conscious or subconscious incentive for the patient to continue to experience symptoms following the surgery. Because both the initial causes of spinal symptoms, as well as the complications of spinal procedures, often involve inflammatory cytokines or mediators, FBSS can be treated, reduced or prevented by the use of TATs as practiced in the invention.

II. Spinal Device or Fusion Procedures

Treatment of spinal disorders generally begins with non-invasive therapies, such as bed rest, non-prescription anti-inflammatory agents and analgesics, injections of cortisone or other non-steroidal anti-inflammatory drugs, traction, bracing and the like. If the pain persists and becomes severe, patients may then undergo further non-invasive or invasive therapies to treat the disorder. Certain invasive therapies include injection of a therapeutic agent, typically steroids, directly into a damaged disk(s). Other invasive therapies involve the use a spinal device or fusion procedure, with or without implantation of a device, or fusion of vertebrae, such as those described below. Typically, as one having ordinary skill in the art will recognize, the recommendation of a particular spinal device or fusion procedure will depend on a variety of factors, including the nature of the particular spinal disorder and its severity and the general health of the patient.

Some invasive spine therapies do not involve implantion of a device or fusion of the vertebrae; see, e.g., co-pending application U.S. Ser. No. ______ (Attorney Docket No. 21782-005001, filed concurrently herewith). For example, standard invasive treatment for HD involves removal of the disk (diskectomy). Standard surgical treatment of SS involves removal or trimming of the lamina of the vertebra, or the ligamentum flavum (laminectomies, laminotomies, and laminoplasties respectively), to widen the spinal canal and create more space for the spinal nerves. Facet joint disease can be treated by radiofrequency neurotomy, and DDD with internal disk derangement by intra-diskal electrothermal therapy (IDET).

Other invasive spine therapies involve the implantation of a device or fusion of the vertebrae, and are described in more detail below.

Disk Nucleus Replacement

Disk nucleus replacement devices are designed to replace the nucleus of a degenerating lumbar disk to alleviate diskogenic and associated pain. These devices help restore disk height and provide the biomechanical properties of the normal nucleus with respect to compressive forces during loading of the spine, bringing the disk back to a more normal physiological function. These devices facilitate the preservation of normal anatomic structures such as the annulus fibrosis, ligaments, and vertebral endplates. Additionally, these devices may delay or prevent facet joint degeneration after diskectomy, and adjacent level disease observed after spinal fusion.

Nucleus replacement devices are intended for use in subjects with mild to moderate DDD or HD to maintain or restore disk height and maintain vertebral segment motion. Use of these devices is less invasive than that of a total disk replacement and complications of total disk replacement like heterotopic ossification are not observed with use of nucleus replacement devices.

Disk nucleus replacement devices are designed to provide the resiliency normally found in a non-degenerated disk while being constrained by the native intact annular material. Disk nucleus replacement implants are designed for placement within the internal space of an inter-vertebral disk, to replace or supplement the function of the normal nucleus pulposus (see, e.g., U.S. Pat. No. 6,620,196).

There are three classes of nucleus replacement devices: hydrogel based, polymeric/synthetic, and mechanical. The hydrogel devices include a hydrogel material that has swelling pressure characteristics of the natural nucleus, implanted in a dehydrated state to minimize disruption of the annulus. The hydrogel then imbibes water and swells to the normal nucleus size, allowing for the reattainment of disk height and the absorption of compressive forces imparted by the adjacent vertebrae during loading. The polymeric/synthetic devices are based on injectable liquids that polymerize in situ in the nuclear cavity when they reach body temperature, and these devices can be used for either total nucleus replacement or to augment a partial diskectomy. The mechanical devices are made of stiffer materials and require a more invasive procedure to implant, and as with the other devices the annulus is required to constrain these devices in the correct anatomical position.

Hydrogel based nucleus replacement implants include, without limitation, Raymedica PDN-SOLO® (U.S. Pat. No. 6,132,465) and Hydraflex® disk replacement with hydrogel core (U.S. Pat. No. 6,533,817), Stryker Aquarelle nucleus replacement with hydrogel (US 20070015178A1), Synthes GeliFlex® hydrogels, CryoLife BioDisc NPR, NuVasive NeoDisc™ nucleus device, and Replication Medical/Abbott Neudisc nuclear replacement device. Polymeric nucleus replacement device materials include, without limitation, Disc Dynamics DasCor® Prosthetic Intervertebral Nucleus (U.S. Pat. No. 7,077,865B2), Sinitec/DePuy Spine Sinux ANR® nucleus replacement (US 20070100349A1), Spine Wave NuCore Injectable Nucleus (U.S. Pat. No. 7,004,945B2), and Gentis—DiscCell™ Nucleus replacement. Mechanical devices include without limitation, EBI Regain® lumbar nucleus replacement, Pioneer NuBac® Surgical Nucleus replacement, and Trans1—PNR nucleus replacement.

Annular Repair Device Implantation

The annulus fibrosis provides a mechanical constraint that allows for the compressive properties of the nucleus pulposus to manifest as a shock absorbing device. The annulus is a highly organized fibrous structure with collagenous bands that impart high tensile properties coupled with a strong interface to the cartilaginous endplates. Disruption of the annulus is the major feature of a herniated disk, with the nucleus pulposus extruding through the defect in the annulus.

Annular repair devices are designed to aid in the repair of tears, fissures, and ultimately herniations in the annulus (observed as HD). In much the same ways as nucleus replacement devices, these devices will help restore normal disk biomechanics and alleviate diskogenic and associated pain. The major features of these devices provide constraints to allow disk height reattainment either in conjunction with nucleus replacements or where a partial diskectomy has been or is being performed. The goal is the preservation of normal anatomic structures such as the ligaments which provide stability to the spine.

These annular repair devices are intended for use in subjects with HD in conjunction with mild to moderate DDD. The goal of these devices is to allow for the restoration of disk height and maintain vertebral segment motion. In some cases, an annular repair device is implanted in subjects who are undergoing a full or partial diskectomy or as an adjunct to a nucleus replacement procedure where the surgeon feels the repair of the annulus is indicated. Repair of the annular structure in conjunction with nucleus augmentation or replacement is believed to be favorable in comparison to proceeding to major surgical techniques like total disk replacement or spinal fusion.

There are at least three approaches for annular repair. One approach uses a mesh device that serves as a scaffold for cellular attachment and proliferation, and subsequent integration into the collagenous repair tissue. Another uses tissue anchors that allow for more efficient surgical closure, particularly in anterior annular tears where the annulus is compressed and surgical closure is challenging. Another technique uses photoactivatable polymeric materials that seal the annular tear.

Annular repair devices include, without limitation, mesh based devices such as Intrinsic Therapeutics Barricaid (US 20040034429A1), surgical anchor based repair devices including Anulex Technologies Inclose (US20060142864A1), and curable polymer-based biomaterials by Endospine, Ltd. (U.S. Pat. No. 6,428,576B1).

Dynamic Stabilization Device Implantation

Dynamic stabilization devices are designed to augment the mechanical stability of the spine and to aid in the decompression of the spinal cord and nerve roots due to SS and mild to moderate DDD. The major features of these devices allow for the preservation of normal anatomic structures such as intervertebral disk, ligaments, and associated structures. These devices allow for restoration of disk height by unloading of the disk and facet joints and are intended to allow for repair of the intervertebral disk, thereby avoiding a spinal fusion. The therapeutic outcome expected with use of these devices is decrease in leg pain and increase in function and quality of life.

Dynamic stabilization devices are intended for use in subjects with degenerative SS of the lumbar spine and/or mild to moderate DDD who are experiencing leg pain (e.g., intermittent neurogenic claudication) due to compression and impingement of the nerve roots. Subjects have usually failed a minimum of six months of conservative therapy and may have had a previous decompression surgery, such as a diskectomy or laminotomy. If subjects have significant DDD or Grade 2 or greater spondylolisthesis, these devices may not be recommended.

Dynamic stabilization devices are designed to preserve or re-establish normal motion of the spine, while re-establishing the normal resting posture of the spine. These devices have several important design characteristics that allow them to function as adjunctive support to the spinal column. There are two major classes of dynamic stabilization devices: pedicle screw based and interspinous spacers. The pedicle screw based devices use flexible rods that allow multiaxial motion of the spine. Interspinous spacers are placed posteriorly and are designed to distract the central spinal canal and foramen, where the nerves branch from the spinal cord into the legs. In addition, subjects having pain originating from the facet joints, ligaments, tendons, or muscles as determined by physical/neurological examination are indicated for dynamic stabilization. SS may also be improved with posterior motion preservation devices. Subjects with moderate to severe SS or mild to moderate DDD may be indicated for dynamic stabilization with an interspinous spacer.

Pedicle screw-based dynamic stabilization devices include, without limitation, Zimmer Spine Dynesys dynamic neutralization system (U.S. Pat. No. 7,073,415), Applied Spine Technologies Stabilimax NZ® (U.S. Pat. No. 7,029,475, 20050182401A1), DePuy Spine Isola/VSP, N Spine N Flex®, Scient'X Isobar®, Interventional Spine Percudyn® percutaneous dynamic stabilization, SpineVision FlexSpine, Triage Medical MIS dynamic stabilization device, Spine Vision X-PLUS, Spine Wave Dynamic Stabilization System, Disc Motion Technologies TrueDyne PDS, Innovative Spinal Technologies Paramount Dynamic Stabilization System, Alphatec Spine Dynamo™ Dynamic Rod, Impliant TOPS™ Total Posterior Arthroplasty System, and Globus Medical's Globus Dynamic Stabilization. Interspinous spacer-based dynamic stabilization devices include, without limitation, Abbott Spine Wallis Stabilization System (U.S. Pat. No. 7,238,204), Paradigm Spine Coflex® (U.S. Pat. No. 5,645,599), Kyphon X-Stop (US 20060271049A1), Privelop's The Spinos, and Globus Medical's Flexus.

Total Disk Replacement (Disk Arthroplasty)

Total disk replacement devices are designed to replace the complete intervertebral disk with a mechanical replacement analogous to the replacement of a hip (i.e., hip arthroplasty). The major features of these devices allow for the preservation of motion in the affected spinal segments and are intended to result in a greater level of pain relief and more complete return to function. These devices require good vertebral body structure with healthy endplates and normal bone quality. These devices attempt to restore normal anatomy and to provide the biomechanical properties of the normal disk with respect to compressive properties. Additionally, these devices may delay or prevent further facet joint degeneration and adjacent level disease by allowing normal motion.

Disk replacement devices are designed to provide the motion normally found in a non-degenerated disk. Some disk devices used polyethylene or metal-on-metal bearing surfaces while newer designs are investigating polymeric bearing surfaces that offer some compressibility and resiliency. Two devices have received recent FDA approval in the US and clinical experiences with these devices have shown some benefits in comparison to fusion procedures.

Disk replacement devices include, without limitation, DePuy Spine Charite Lumbar Disk and Discover Cervical Disk (US 20060178745A1, US 20060004452A1), Synthes ProDisc and ProDisc C (U.S. Pat. No. 6,726,720), Medtronic Sofamor Danek Maverick Artificial Disk and Prestige® LP Cervical Disc (U.S. Pat. No. 6,740,118B2, U.S. Pat. No. 6,899,735B2), Stryker Spine Cervicore® Cervical Disc and Flexicore® Lumbar Disc, Blackstone Medical Pillar disk, LDR Spine Mobidisc and Mobidisc C, Ranier Technology CAdisc-L and CAdisc-C, US Spinal Technologies Spartacus Artificial Disc, Disc Motion Technologies TrueDisc PL, MedicineLodge Infinity Disc, NuVasive Neodisc™ Cervical Disc, Spineart Baguerac® disk, Pioneer Surgical Technology Nubac™ Disc Arthroplasty System, SpinalMotion Kineflex Lumbar Disc and Kineflex C Cervical Disc, Aesculap Spine Activ L disk, SpinalKinetics M6° Artificial Cervical Disc, Globus Medical Alliance TDR and Secure-C, Biomet Spine Regain disk, and Cervitech's PCM & PCM-V disk replacement system.

Spinal Fusion

Spine fusion surgery devices are designed to provide biomechanical stability to the spine segments for one or multilevel fusion surgery. Spinal fusion refers to the growing of a continuous bony bridge between two vertebrae to convert two motion segments into one motion segment. These devices can restore the normal anatomical curvature of the spine (e.g. lordotic fusion cages) and provide osteoconductive surfaces for the new bone growth required to fuse the vertebral segments. Fusion devices are intended for use in subjects with spinal instability from SLD; SS; severe DDD with internal disk derangement and diskogenic pain; and persistent radicular pain.

There are two major classes of contemporary fusion procedures performed, intervertebral body fusion and posterolateral (or interspinous process) fusion. Interbody spinal fusion is accomplished by performing a total diskectomy, decorticating the cartilaginous endplates of the adjacent vertebral bodies, and then placing between the vertebral bodies either fusion cages or a machined allograft that restores spacing between the vertebrae and allows for bone growth in and around the devices. Mechanical stabilization may be improved or augmented with the use of pedicle or facet screws, or plates. In the case of posterolateral fusion, bone graft material is placed along the lateral gutters of the interspinous process and pedicle screws are used for mechanical stabilization. Posterolateral fusion procedures are not indicated for subjects with severe spinal instability.

Interbody fusion devices include, without limitation, Medtronic Sofamor Danek LT Cage (U.S. Pat. No. 6,375,655), DePuy Spine Jaguar I/F Cage (Radiolucent, U.S. Pat. No. 7,229,477), Zimmer Spine BAK® Cage (U.S. Pat. No. 6,270,498), Stryker Spine Ray Threaded Fusion Cage® (U.S. Pat. No. 5,658,337), Biomet Spine NeoLif® Intersomatic Lumbar Cage, Spine Wave StaXx™ XD System, SpineVision Spacevision ACIF Cage, US Spine PEEK Cage, and LDR Spine MC+® and ROI® Fusion System. Pedicle screw systems include, without limitation, Medtronic Sofamor Danek CD Horizon System (U.S. Pat. No. 6,783,527B2), DePuy Spine ISOLA® Spinal System (U.S. Pat. No. 6,080,156) and Monarch Pedicle Screw System, Abbott Spine BacFIX® Fixation System, Biomet Spine SpineLink® II, Zimmer Spine Optima ZS Pedicle Screw System, Custom Spine ISSYS™ Pedicle Screw System, US Spine Pedicle Screw System, SpineVision X-Plus and Uni-Thread™ Pedicle Screw System, LDR Spine Easyspine® posterior pedicle screw system, Innovative Spinal Technologies Paramount™ Pedicle Screw System, Blackstone Medical/Orthofix ICON™ Spinal Fixation System, and K2M MESA™ Spinal System. Allograft fusion cages include, without limitation, Medtronic Sofamor Danek Tangent Lumbar Cage (U.S. Pat. No. 6,989,031B2), DePuy Spine VG1 ALIF Allograft, Abbott Spine Allofuse, Biomet Spine OsteoStim™ PLIF, and US Spine Allograft Cage. Plate fixation systems include, without limitation, Medtronic Sofamor Danek Atlantis Plate System (U.S. Pat. No. 7,004,944B2), DePuy Spine Swift Dynamic Cervical Plate (US 20050209593A1), Abbott Spine SC AcuFIX® Cervical Plate, Biomet Spine VueLock®, SpineVision C3 Anterior Cervical Plate, Inion S-1™ Biodegradable Anterior Cervical Fusion System, and Blackstone Medical/Orthofix Hallmark™ Anterior Cervical Plate System. Vertebral Body Replacements include, without limitation, Medtronic Sofamor Danek VERTE-SPAN® (US 20060084975A1), DePuy Spine BENGAL™ System (U.S. Pat. No. 6,569,201), Abbott TraXIS® VBR, and LDR Spine MC+® and ROI® Partial Vertebral Body Replacement System.

Spine Fusion with Growth Factor Augmentation

The above techniques for interbody and posterolateral fusion can be augmented with the use of bone growth stimulatory proteins or peptides, which replace the need for an autogenous iliac crest autograft. The first growth factor approved by FDA for spinal fusion was bone morphogenetic protein-2 (BMP-2); it is used in interbody fusion procedures. The BMP-2 protein is absorbed on a collagen sponge, and placed inside an interbody fusion cage, which is then threaded between two vertebral bodies. The release of BMP-2 stimulates bone formation in and around the fusion cage, yielding successful fusion of the two vertebral bodies into one motion segment. These proteins are also being investigated for repair and regeneration of the nucleus of the intervertebral disk. Other cellular based (e.g., stem cell) therapies are also being developed for spinal fusion applications, and their intended use is to replace or supplement an autograft.

Bone growth stimulatory proteins and peptides and devices incorporating the same, include, without limitation, Medtronic Sofamor Danek Bone Morphogenetic Protein-2 (InFuse, U.S. Pat. No. 7,172,629B2, U.S. Pat. No. 6,150,328), Stryker Spine Osteogenic Protein-1 (OP-1, U.S. Pat. No. 7,176,284), DePuy Spine Growth Differentiation Factor 5 (GDF-5), BioSET B2A Peptide combined with HA/TCP (Amplex), Acologix Bone Growth Protein (AC-100), Bonebiologics Bone Growth Protein (UCB-1), and Scil STO 1 Spine Fusion Device. Cellular therapies include, without limitation, Aastrom Biosciences Tissue Repair Cells, Osiris Therapeutics Cellular Therapy Osteocell, and Blackstone/Orthofix Bone Marrow Cell Product Trinity.

Kyphoplasty/Vertebroplasty/Vertebral Restoration

Kyphoplasty and Vertebroplasty are techniques used to restore the height of a fractured vertebral body (vertebral compression fracture (VCF)). These fractures are diagnosed by x-ray or CT and are correlated with acute back pain at the site of the vertebral compression fracture. The Kyphoplasty or vertebroplasty procedure entails drilling into the vertebral body, inserting a balloon catheter, and inflating the balloon until the height of the vertebrae is restored to normal. A flowable and hardenable material, such as polymethylmethacrylate cement, is then injected into the void space, which sets up quickly, thereby preventing the vertebrae from collapsing. The normal outcome of this procedure is an immediate reduction in back pain due to the vertebral compression fracture.

Kyphoplasty and vertebroplasty systems include, without limitation, Kyphon Balloon Kyphoplasty System (U.S. Pat. No. 6,248,110B1), SpineWave StaXx FX System, and Parallax Medical EZflow Vertebroplasty System.

Facet Repair and Replacement

Facet repair and replacement devices are designed to replace the facets that are damaged due to facet arthritis (degeneration of the facet joint). There are two types of facet replacement devices, pedicle screw based and spacer based. The pedicle screw based systems include, without limitation, Archus Orthopedics Total Facet Arthroplasty System® (U.S. Pat. No. 7,051,451), Facet Solutions Anatomic Facet Replacement System® (U.S. Pat. No. 7,041,136), and Impliant TOPS Total Posterior Arthroplasty System. The malleable spacer-based device includes, without limitation, the Spinal Elements Zyre™ Facet System.

Anti-Adhesion Device Implantation

The methods of the current invention can be used to prevent the need for, or to enhance the outcome of any spinal procedure that is performed with an anti-adhesive device. Epidural fibrosis is the formation of fibrotic scar tissue around/near the nerve root associated with a spinal device or fusion procedure, which can cause recurring leg and back pain. Symptomatic epidural fibrosis occurs following approximately 10% of spinal device or fusion procedures. It is a significant cause among the many factors that can result in FBSS. Binding of lumbar nerve root fibers by fibrous adhesions is believed to be the mechanism by which epidural fibrosis causes recurring pain.

Anti-adhesion devices (gels, sealants, and/or barriers) can be used during any of the spinal device or fusion procedures described herein, to reduce or prevent formation of fibrous adhesions, thereby improving the outcome of a spinal device or fusion procedure. Anti-adhesion devices could also be used with spinal device or fusion procedures that do not involve the implantation of a device, or fusion of two or more vertebrae, such as for example diskectomy or laminectomy procedures, percutaneous or endoscopic epidural adhesiolysis, radiofrequency neurotomy (RFN), or intradiskal electrothermal therapy (IDET); see, e.g., co-pending U.S. application Ser. No. ______ (Attorney Docket No. 21782-005001), filed concurrently herewith.

Current treatment options for epidural fibrosis are limited. Epidural adhesiolysis is used to treat patients with persistent pain following laminectomy or back surgery and sometimes for patients with persistent long-term back pain that has failed other conservative or non-surgical interventional pain procedures. The conditions treated with epidural adhesiolysis include epidural fibrosis and adhesive arachnoiditis which may result in NR entrapment or irritation. The latter conditions rarely occur in the absence of previous surgical, and more often multiple, surgical interventions while epidural fibrosis may rarely occur without previous surgery and accounts for some of the beneficial results seen with this technique in patients who have not had surgery. Post surgery, persistent symptoms can lead to a diagnosis of post-laminectomy syndrome, or FBSS, which is the more usual criteria for a trial of epidural adhesiolysis. Anti-adhesion devices include, without limitation, Fziomed Oxiplex® SP Gel (U.S. Pat. No. 6,869,938B1), Tyco/Confluent Surgical DuraSeal Xact™ (U.S. Pat. No. 7,220,270B2), Integra Life Sciences Duragen Plus®, and Biomet Mesofol® Absorbable Film.

New/Adjunctive Technologies for Spinal Surgery

Other technologies can be used in conjunction with many of the spinal device or fusion procedures described above. Thus, subjects that are eligible for any of the spinal device or fusion procedures described previously are eligible for use of an adjunctive technology described herein. For example, the success rate of spinal fusion procedures varies according to many factors, and non-invasive bone growth stimulation devices (BGS) have been developed as adjunctive technologies to aid in the rate and overall success of fusion surgery. These devices include, without limitation, Biomet Spine SpinePak BGS, Orthofix SpinalStim BGS Device, and DJ Orthopedics SpinaLogic BGS Device. Surgical procedures may have to be performed to repair the dura; useful devices include, without limitation, Pegasus Biologics DurADAPT™ dural repair system and Kensey Nash Dural Repair Device. Anterior spinal device or fusion procedures can have complications with vessels that are in the surgical site, which can be prevented with devices including, without limitation, the Gore Preclude Vessel Guard.

III. Methods for Identifying Subjects Eligible for Spinal Device or Fusion Procedures

As indicated previously, the inventor has discovered that patients who are suffering from moderate to severe disorders of the spine, as described previously, and that are eligible for a spinal device or fusion procedure, as described above, are candidates for treatment with TATs to prevent, delay, or improve the outcome of the invasive procedure. Such identification of these patients as eligible for treatment with a TAT is surprising. The current standard of care does not teach administration of a TAT to patients eligible for such spinal device or fusion procedures. Such patients may be offered epidural steroids. If the steroids fail to resolve the pain, the patients are typically treated with surgery. It is typically thought that such patients will not benefit from administration of a currently approved TAT, such as the TNF inhibitors Enbrel® (etanercept), Humira® (adalimumab), and Remicade® (infliximab).

Accordingly, this disclosure provides a method of identifying a subject that could benefit therapeutically from administration of a TAT, such as a direct TNF inhibitor (direct TNF-I). The method includes determining that the subject meets the eligibility criteria for at least one predetermined SOE for a spinal device or fusion procedure, thereby identifying the subject as one who could benefit.

A. General Eligibility Criteria

The identification of a subject as one that would benefit therapeutically from treatment with a TAT is based on the subject meeting the eligibility criteria in at least one (e.g., 1, 2, 3, 4, or more) of the predetermined SOE(s) for a spinal device or fusion procedure. Because many of the devices and device procedures are still experimental emerging therapies under active development, the clinical eligibility criteria are often not captured in widely available CPGs, but rather in the eligibility criteria for patients to enter a clinical trial of the device or procedure. Such SOEs, including clinical trial eligibility criteria, and the availability of CPGs and the specific clinical eligibility criteria in the CPGs, will change with development of the emerging therapies, changing healthcare practice and treatment options, and may vary from country to country. As SOEs including CPGs change, a skilled healthcare provider will be able to determine which patients are eligible for the spinal device or fusion procedure, relying upon professional judgement, upon eligibility criteria for clinical trials, as well as the results of the clinical trials when available, upon CPGs generated by the provider's own healthcare organization, upon externally generated CPGs, and upon other guides to the current professional standard of care governing determination of eligibility for spinal device or fusion. A skilled healthcare provider will also be able to identify a currently relevant predetermined SOE, including a CPG. The predetermined SOEs including CPGs and clinical trial eligibility criteria referenced herein are not meant to be all encompassing, nor will they remain static. They are illustrative of current predetermined SOEs, CPGs and clinical trial eligibility criteria for spinal device and fusion procedures.

A predetermined SOE could include, for example:
a) a determination of eligibility of the subject for the spinal device or fusion procedure by a healthcare service provider (e.g., a physician, physiatrist, osteopathic physician, physician's assistant, nurse practioner, physical therapist, nurse, or other qualified allied health professional), for example according to the healthcare provider's clinical judgement, according to a CPG internally generated by the healthcare organization in which the provider practices, according to an externally generated CPG, or according to the eligibility criteria for a clinical trial of the device or fusion procedure. Thus, the healthcare service provider has determined that the subject meets that provider's own criteria for undergoing the spinal device or fusion procedure, as evidenced by one or more of the following:
   i) a scheduling or request for scheduling by a healthcare service provider of the spinal device or fusion procedure for the subject. The fact that the procedure has been scheduled or requested for scheduling indicates that the healthcare service provider deems the subject to meet its criteria for undergoing the procedure;
   ii) a communication by a healthcare service provider to the subject that the subject has been determined to be eligible for the spinal device or fusion procedure. As above, the communication by the healthcare service provider indicates that the healthcare service provider deems the subject to meet its criteria for undergoing the procedure;
   iii) a provision to, or offering to the subject by a healthcare service provider of a consent form for the spinal device or fusion procedure, or of an informed consent form for a clinical trial of the spinal device or fusion procedure. As above, the provision, offering, or receipt indicates that the provider deems the subject to meet its criteria for undergoing the procedure;
   iv) a receipt or execution by the subject of a consent form for the spinal device or fusion procedure, said consent form provided by the subject's healthcare provider, or of an informed consent form for a clinical trial of the spinal device or fusion procedure. The fact that the subject has received and/or executed a consent form provided by the subject's healthcare provider, or an informed consent form for a clinical trial of the device or procedure, indicates that the subject must be eligible for the procedure;
   v) a notation by the healthcare service provider in a tangible medium such as the patient's written or electronic medical record, or in the eligibility screening records for a clinical trial, that the patient is eligible for the spinal device or fusion procedure. The fact that the provider has made such a notation of eligibility indicates that the subject must be eligible for the procedure.
b) a determination of eligibility of the subject for the spinal device or fusion procedure by a qualified entity other than the subject's healthcare provider or clinical trial provider, such as a healthcare provider organization [including a hospital, a health maintenance organization, a (HMO), a managed care organization, a defined healthcare provider network, or group practice], a national or local healthcare system, a hospital review committee, a professional guidelines committee, or a healthcare reimbursement agency, an insurance provider, or any other 3^{d} party payor. The approval by one of the listed parties indicates that the subject meets a set of criteria set forth by the same to undergo the procedure, and is therefore eligible for the procedure;
c) the meeting by the subject of the eligibility criteria for a spinal device or fusion procedure in one or more CPG(s) governing eligibility for a spinal device or fusion procedure, generated by, for example: a healthcare service provider organization including a hospital, a health maintenance organization, a managed care organization, a group practice, or a defined healthcare provider network; a professional organization of healthcare providers such as, for example, North American Spine Society (NASS), American Academy of Orthopedic Surgeons (AAOS), or American Society of Interventional Pain Physicians (ASIPP); a healthcare reimbursement agency; a national or local healthcare system; a hospital review committee; a professional guidelines committee; or a 3^{rd} party payor.
d) the meeting by the subject of the eligibility criteria for entrance into a clinical trial of the device or procedure.

In some embodiments, to be eligible, a subject will further have failed to have achieved long term or sufficient relief from pain from a previous spinal device or fusion procedure, including a decompression (e.g., a partial or complete diskectomy or laminectomy) procedure or a device or fusion procedure.

B. SOEs for Spinal Device or Fusion Procedures

Clinical eligibility criteria for particular spinal device or fusion procedures are set forth in Table 1 and described in more detail below, with reference to illustrative CPGs and/or clinical trials of the spinal device or fusion procedure.

TABLE 1

| | |
|---|---|
| Spinal Device or Fusion | |
| Procedure [reference(s)] | SOE |

Part A

| | |
|---|---|
| Nucleus Replacement [5] | 1) One of the following: |
| | a) HD confirmed on MRI, or |
| | b) Mild to moderate DDD confirmed on MRI with loss of disk |
| | height <50%; |
| | AND |
| | 2) a) Failed conservative treatment for at least 6 weeks |
| | b) Back or leg pain suggestive of L2 - S1 NR involvement, |
| | or radicular neck pain |
| | c) Absence of facet arthropathy, SS, or spinal segment instability |
| | (SLD) |
| Annular repair surgical | 1) Both of the following: |
| procedure [6] | a) Eligible for lumbar or lumbo-sacral diskectomy, e.g. HD, MRI |
| | confirmation of herniation at right location to explain associated leg |
| | pain[4]; and |
| | b) Failed conservative treatment for 6 to 12 weeks; |
| | OR |
| | 2) a) Patient undergoes nucleus replacement procedure; and b) |
| | surgeon elects conjoint annular repair. |
| Dynamic stabilization with | 1) One, two or three of the following: |
| pedicle screw based device [7, 8] | a) Mild to moderate DDD confirmed on MRI with either back or leg |
| | pain from L2 - S1 |
| | b) SS (confirmed by CT and/or MRI) with either back/leg pain from |
| | L2 - S1 |
| | c) Pain originating from the disk, facet joints, and/or ligaments |
| | confirmed by physical/neurological examination; |
| | AND |
| | 2) Failed conservative treatment for at least 6 months. |
| Dynamic stabilization with | 1) One of the following: |
| interspinous spacer [9, 10] | a) Moderate to severe SS confirmed by CT and/or MRI |
| | OR |
| | b) Mild to moderate DDD confirmed on MRI; |
| | AND |
| | 2) One of the following: |
| | a) Intermittent neurogenic claudication (leg pain and impaired |
| | function) |
| | b) low back pain improving with flexion, and |
| | c) radicular leg pain. |
| | AND |
| | 3) Failed conservative treatment for at least 6 months. |
| Artificial disk | 1) Moderate to severe DDD confirmed by MRI with provocative |
| [11, 12] | discography and |
| | 2) Back, leg, shoulder, or arm pain; |
| | AND |
| | 3) Absence of: |
| | a) Severe facet arthropathy |
| | b) Gross spine instability |
| | c) Vertebral body osteoporosis |
| | AND |
| | 4) Failed conservative treatment for minimum of 6 months (lumbar) |
| | and 6 weeks (cervical). |

Part B

| | |
|---|---|
| Interbody spinal fusion | 1) DDD with one or more of the following: |
| [13] | a) moderate to severe spinal instability, |
| | b) SS, |
| | c) Spondylolisthesis, all confirmed by CT, MRI, and/or x-ray |
| | AND |
| | 2) Back or neck pain that has failed conservative treatment for a minimum |
| | of 6 months. |
| Posterolateral spinal | 1) One or both of the following: |
| fusion [14] | a) DDD w/degenerative spondylolisthesis confirmed by MRI and/or CT; |
| | b) SS confirmed by CT and/or MRI; |
| | AND |
| | 2) Low back pain that has failed conservative treatment for minimum of 6 |
| | months. |
| Interbody spinal fusion | 1) DDD with one or more of the following: |
| with BMP-2 [15, 16] | a) moderate to severe spinal instability, |
| | b) SS confirmed by CT and/or MRI, |
| | c)spondylolisthesis, all confirmed by CT, MRI, and/or x-ray |
| | AND |
| | 2) Chronic back pain that has failed conservative treatment for a minimum |
| | of 6 months |
| Kyphoplasty/ | 1) Vertebral compression fractures confirmed on standard x-ray, CT and/or |
| vertebroplasty [17, 18] | MRI; |
| | AND |
| | 2) Back pain correlated with site of vertebral compression fractures. |
| Facet Replacement | 1) Diagnosis of facet arthritis on CT or MRI |
| Surgery [19] | optionally with |
| | 2) Degenerative confirmed by CT and/or MRI; |
| | AND |
| | 3) Intermittent neurogenic claudication (leg pain and impaired function) that |
| | worsens on walking or standing with radiological evidence of nerve root |
| | impingement. |
| Spine Surgical Procedure | Eligibility for: |
| using Anti-adhesion gel or | 1) any of the spinal device or fusion procedures described above; |
| barrier to prevent | OR |
| epidural fibrosis | 2) any spinal device or fusion procedure that does not involve the |
| [20, 21] | implantation of an implantable device or fusion device, such as diskectomy |
| | or laminectomy procedures, percutaneous or endoscopic epidural |
| | adhesiolysis, RFN, or IDET; see, e.g., co-pending Application U.S. Ser. No. |
| | (Attorney Docket No. 21782-005001), filed concurrently |
| | herewith). |

To be eligible for a disk nucleus replacement device spinal device or fusion procedure, subjects must have been diagnosed with: 1) a) HD confirmed on MRI or b) mild to moderate DDD confirmed on MRI with a loss of disk height of less than 50 percent; and 2) a) have failed conservative treatment for a period of at least 6 weeks; b) have back or leg pain from L2-S1 with nerve root involvement or radicular neck pain; and c) have absence of facet arthropathy, SS, or spinal segment instability.

To be eligible for an annular repair device spine invasive procedure, subjects must have been diagnosed as having 1) a) HD with MRI confirmation and have associated leg pain; and b) have failed conservative treatment for a period of at least 6 weeks; or 2) a) subjects are undergoing nucleus replacement and b) treating spine interventionalist elects to perform conjoint annular repair.

To be eligible for an invasive dynamic stabilization spinal procedure with pedicle screw based device, subjects must have been diagnosed with 1) one, two, or three of the following: a) mild to moderate DDD and/or b) moderate to severe SS with back or leg pain from L2-S1 (both would be confirmed by MRI and/or CT) and/or c) pain originating from the disk, facet joints, and/or ligaments confirmed by physical/neurological examination; and 2) have failed conservative treatment for a period of at least 6 months.

To be eligible for an invasive dynamic stabilization spinal procedure with an interspinous process spacer based device, subjects must have been diagnosed with one of the following: 1) a) mild to moderate DDD or b) moderate to severe SS with back or leg pain from L2-S1 (both would be confirmed by MRI and/or CT); and 2) a) intermittent neurogenic claudication, or b) low back pain improving with flexion, or c) radicular leg pain; and 3) have failed conservative treatment for a period of at least 6 months.

To be eligible for disk replacement with an artificial disk, subjects must have been diagnosed with moderate to severe DDD confirmed by MRI. For lumbar applications, subjects also experience back or leg pain with provocative diskography. Subjects have failed at least 6 months of conservative therapy. For cervical applications, subjects must have been diagnosed with DDD with radiculopathy manifesting as neck or arm pain or a decrease in muscle strength. Clinical symptoms in these cervical subjects are correlated with radiologic findings on CT or MRI and these subjects may have failed conservative therapy for a minimum of 6 weeks. Subjects must have absence of severe facet arthropathy, gross spine instability, and vertebral body osteoporosis.

To be eligible for an interbody spinal fusion procedure, subjects must have been diagnosed with 1) DDD and one or more of the following: a) moderate to severe spinal instability, and/or b) SS, and/or c) spondylolisthesis; all of which have been confirmed by either CT, and/or MRI, and/or x-ray; and 2) have back or neck pain that has failed conservative treatment for a minimum of 6 months.

To be eligible for a posterolateral fusion, subjects must have been diagnosed with 1) a) DDD with degenerative spondylolisthesis and/or b) SS confirmed by MRI and/or CT; and 2) have low back pain that has failed conservative treatment for a period of at least 6 months.

To be eligible for an interbody spinal fusion procedure using BMP-2, subjects must have been diagnosed with 1) DDD and one or more of the following: a) moderate to severe spinal instability, b) SS, and/or c) spondylolisthesis, all of which have been confirmed by either CT, and/or MRI, and/or x-ray; and have 2) back pain that has failed conservative treatment for a minimum of 6 months.

To be eligible for kyphoplasty, vertebroplasty or vertebral restoration, subjects must have been diagnosed with 1) a vertebral compression fracture confirmed on x-ray, CT and/or MRI; and 2) experience back pain correlated with the site of the vertebral compression fracture.

To be eligible for facet replacement procedures, subjects must have been 1) diagnosed with facet arthritis confirmed by CT and/or MRI and optionally with 2) degenerative SS; and 3) experience intermittent neurogenic claudication (e.g., leg pain and impaired function) that worsens on walking or standing, coupled with radiological evidence of nerve root impingement by either osseous or non-osseous elements.

To be eligible for a spinal device or fusion procedure with concomitant implantation of an anti-adhesion gel or barrier, subjects must be eligible for 1) any of the spinal device or fusion procedures described above; or 2) any spinal device or fusion procedure that does not involve the implantation of an implantable device or fusion of vertebrae, such as diskectomy or laminectomy procedures, percutaneous or endoscopic epidural adhesiolysis, RFN, or IDET. For eligibility criteria for such procedures, see co-pending U.S. application Ser. No. ______ (Attorney Docket No. 21782-005001), filed concurrently herewith.

Once a subject has been identified as eligible, this identification can be further transmitted, e.g., to a healthcare service provider. The identification can also be memorialized, e.g., in a tangible medium of expression such as a patient's physical chart or record or a computer readable database. In some cases, the identification can be communicated to the subject, e.g., in the form of a recommendation that the subject undergo treatment with a TAT. In some cases, the subject will subsequently undergo treatment, e.g., administration of a TAT, according to any of the methods as disclosed further herein.

IV. Methods for Preventing or Postponing a Spinal Device or Fusion Procedure

Also provided herein are methods for treating a subject, e.g., preventing, reducing, delaying, eliminating, or postponing a subject's need for or eligibility for a spinal device or fusion procedure, where the subject meets the eligibility criteria for at least one predetermined SOE for a spinal device or fusion device or fusion procedure. The method includes: a) optionally identifying the subject as a subject eligible for the spinal device or fusion procedure, e.g., according to the methods described previously; and b) administering to the subject a therapeutically effective amount of at least one TAT, e.g., a direct TNF-I.

If a subject is optionally identified, then the identification can be further transmitted, e.g., to a healthcare service provider. The identification can also be memorialized, e.g., in a tangible medium of expression such as the patient's physical chart or record or a computer readable database. In some cases, the identification can be communicated to the subject, e.g., in the form of a recommendation that the subject undergo treatment with a TAT.

Any TAT including those as described more fully below can be employed in the methods. Any combination of TATs can be used in the methods, e.g., 2, 3, 4, or more TATs can be used in the methods. Similarly, any administration regimen or route can be employed in the methods, including those described below.

In some cases, the effect of administering the TAT can be assessed to determine if the subject's eligibility for the spinal device or fusion procedure has been eliminated, prevented, delayed, reduced, or postponed. An assessment of the effect of an administration of TAT can be performed by methods known to those having ordinary skill in the art, such as the methods used to diagnose and/or determine eligibility for the spinal device or fusion procedure. Non-limiting examples of methods used to assess the effects of administration of a TAT can include:

a) determination of the level or temporal duration of pain, degree of impaired mobility, or signs of spinal nerve root irritation in the subject as previously documented on physical examination, radiologic, or electrodiagnostic studies, compared to baseline characteristics;

b) determination of the amount of a cytokine of interest, e.g., TNF (such as soluble TNF) in the subject (e.g., in a location of interest, such as a disk);

c) fluoroscopically or radiologically observing the subject (e.g., to evaluate the spinal disorder); and

d) re-evaluation of the history, physical exam, radiologic, and other criteria that rendered the patient eligible for the procedure, in order to determine whether the subject continues to meet the eligibility criteria in the SOE, CPG, or clinical trial of the spinal device or fusion procedure.

Determining a level or duration of pain in a subject can be done using standard objective and subjective methods known to those having ordinary skill in the art, including methods employed to diagnose and/or determine eligibility for the spinal device or fusion procedure. Determining the amount of a cytokine of interest can also be performed using standard assays, such as bioassays, ELISA-based assays (e.g., ELISPOT assays), HPLC assays, and MS assays. Samples for measurement can be obtained from a location of interest, e.g., local to an HD or site of stenosis, including intradiskal biopsy samples.

Fluoroscopic or radiologic (e.g., MRI, X-ray, CT) observations can be performed using methods known to those having ordinary skill in the art. Typically the site observed will correlate with the location of the HD, SS or other spinal pathology.

In some case, the results of any of the assessment methods can be compared with a similar assessment performed prior to administration of the TAT. Multiple assessments during a course of TAT administration are also contemplated, e.g., 2, 3, 4, 5, 6 or more temporally separate assessments. Any suitable amount of time between assessments can occur, and can be determined by one having ordinary skill in the art. In some embodiments, from about 1 hour to about 2 months, or any time there between, elapses between assessments (e.g., 1 day, 2 days, 5 days, 1 week, 2 weeks, 4 weeks, 6 weeks, 8 weeks, or 2 months). Typically, one might expect to witness a response within about five half lives of a TAT or within about 2-8 weeks after initial administration of the TAT.

Administration of a TAT can include more than one administration of a TAT, e.g., at least 2, 3, 4, 5, 6 or more separate administrations of the TAT. The appropriate duration of time elapsed between the first and second (or any subsequent) administration of a TAT can be determined by one having ordinary skill in the art and may be determined based on the subject's need (e.g., pain level, responsiveness to the TAT, etc.), the route and regimen of administration; and an assessment of the effect of the first administration. Typically the time elapsed between administrations can range from about 1 day to about 2 months, or any time there between (e.g., 3 days, 5 days, 10 days, 20 days, 30 days, 45 days, 60 days). If a subject experiences a beneficial response from injection of a TAT, which has prolonged benefit (defined as one month or longer) and then experiences renewed symptoms anytime after this period, from 2 months to 20 or more years later, the administration of the TAT can be repeated in similar manner to the initial administration.

An administration of a TAT according to the methods described herein can treat the subject so that the subject does not undergo a spinal device or fusion procedure in the period following the TAT administration, ranging from the following 1-12 months (e.g., 1, 2, 3, 4, 5, 6, 7, 8, 9, 10, 11, or 12 months) to the following 1-20 years (e.g., 1, 2, 3, 4, 5, 6, 7, 8, 9, 10, 11, 12, 14, 16, 18, or 20 years) after the initial administration of the TAT. In some cases, the subject does not undergo the spinal device or fusion procedure, and thus the method has prevented or eliminated the need for the spinal device or fusion procedure.

IV. Methods for Improvement of Outcome of Spinal Device or Fusion Procedures

Any spinal device or fusion procedure, whether diagnostic or therapeutic, may disrupt and damage the disk and surrounding tissues. Such tissue disruption, by releasing inflammatory cytokines including TNF, can further inflame and damage the nearby nerve roots, peripheral nerves, and other adjacent tissues. Thus, spinal device or fusion procedures can inadvertently exacerbate as well as relieve a subject's symptoms and disability. Furthermore, spinal device or fusion procedures are not always successful in the long term. In some patients, the procedures initially alleviate the subject's symptoms, only to recur and progress, sometimes necessitating repeat surgery with a less favorable likelihood of success.

The inventor has discovered that subjects that are eligible for and undergo a spinal device or fusion procedure, including those subjects who have been previously administered a TAT as described herein (e.g., to prevent, eliminate, postpone, delay, or reduce the need for the procedure), can also benefit therapeutically from administration of a TAT. Thus, a TAT administration initially provided to prevent, delay, or reduce the need for an invasive procedure can improve the therapeutic outcome of a subject who eventually undergoes the procedure. In other cases, such an initial TAT administration may not be performed, but an administration of the TAT is coordinated to occur peri-operatively, e.g., at a time period prior to, during, and/or after the spinal device or fusion procedure, in order to improve the therapeutic outcome of the subject. For example, in some cases, a healthcare service provider may administer a TAT peri-operatively in order to reduce inflammation in a region of a spinal disorder. In such situations, the provider may have determined that the subject was eligible for the procedure and may have decided to proceed with the procedure, with the understanding that the subject would receive one or more administrations of a TAT peri-operatively. In yet other cases, both an initial TAT administration to prevent, delay, or reduce the need for the invasive procedure and a peri-operative administration are employed to improve the therapeutic outcome of a subject that does ultimately undergo the procedure.

Thus, in some embodiments, the present disclosure provides a method for improving a subject's outcome from a spinal device or fusion procedure, where the subject meets the eligibility criteria for at least one predetermined SOE for a spinal device or fusion procedure. The method can include:

a) optionally identifying the subject as a subject eligible for the spinal device or fusion procedure;

b) administering to the subject a therapeutically effective amount of at least one TAT; and

c) performing the spinal device or fusion procedure.

The administration of the TAT can be by any method as described herein, and can include more than one TAT. In some invasive procedures, a device may be implanted that can release a TAT itself, e.g., a coated device, a device that comprises a depot, hydrogel, or a controlled-release formulation, or a device that includes a reservoir that dispenses a TAT. For example, as described further below, annular repair or replacement devices, dynamic stabilization devices, spinal fusion devices, kyphoplasty/vertebroplasty/vertebral restoration devices, facet replacement and fixation devices, and dural repair devices can be implanted that can administer the TAT themselves, e.g., via a coating, depot, reservoir, or controlled-release formulation or hydrogel.

In some embodiments, the administration can include administering a TAT that is in addition to the TAT administered by the device, e.g., via a route of administration other than or in addition to the route of administration of a TAT by the implanted device itself. In yet other embodiments, the administration of the TAT may involve the use of an induction-maintenance regimen, as described herein.

In other embodiments, the method can include:

a) optionally identifying the subject as a subject eligible for the spinal device or fusion procedure;

b) administering to the subject a therapeutically effective amount of at least one TAT (e.g., prior to and/or during the invasive procedure);

c) performing the invasive procedure; and

d) optionally administering to the subject a therapeutically effect amount of at least one TAT after the invasive procedure.

In all such embodiments, the present methods contemplate the administration of a TAT that is in addition to the TAT administered via a device itself. Thus, a method for improving the outcome of a subject from a spinal device or fusion procedure, where the spinal device or fusion procedure implants a device that can administer a TAT, is provided. The method can include:

a) optionally identifying the subject as a subject eligible for the spinal device or fusion procedure;

b) performing the spinal device or fusion procedure, wherein a device that delivers a TAT is implanted; and

c) administering to the subject a therapeutically effect amount of at least one TAT in addition to the TAT that is administered via the implanted device.

The additional administration of the TAT can be at any time relative to the spinal device or fusion procedure, e.g., peri-operatively, such as before, during, and/or after the spinal device or fusion procedure. The additional administration can involve an induction and maintenance regimen as described herein.

Administration of the TAT in any of the above methods can be performed using any route or regimen of administration, as described herein, including multiple administrations of one or more TATs. Administration of a TAT can be prior to, during, and/or after the spinal device or fusion procedure. The administration of a TAT prior to, during, and/or after the spinal device or fusion procedure can be in addition to an administration of a TAT completed prior to the spinal device or fusion procedure, e.g., an administration that delayed or postponed the spinal device or fusion procedure.

To address any perceived risk of increased infection risk upon administration of a TAT peri-operatively, the inventor has provided novel regimens of administration in which a TAT can either be administered locally, to reduce systemic exposure and infection risk, and/or can be optionally interrupted, e.g., for a time period prior to and/or after the spinal device or fusion procedure, with resumption of the TAT treatment regimen post-operatively. Peri-operative interruption of therapy would be at the discretion of the clinician responsible for managing the patient's therapy before, during, and/or after the spinal device or fusion procedure. The optional interruption time period prior to and/or after the spinal device or fusion procedure can be about equivalent or can be different. An optional interruption time period can range from about 1 day to about 14 days, or any time there between (e.g., 2, 4, 6, 8, 10, 12 days). In some embodiments, the optional interruption time period prior to and/or after the spinal device or fusion procedure is equivalent to about 1 to about 4 half-lives (t_{1/2}) (e.g., 1, 2, 3, or 4 half-lives) of the TAT in serum. Typically, the optional interruption period will be longer prior to the invasive procedure than after the invasive procedure.

The therapeutic outcome of the subject from the spinal device or fusion procedure can be improved, e.g., based on the administration of the TAT. An improvement in therapeutic outcome can be determined by methods known to those having ordinary skill in the art and can include at least one of the following:

a) a reduction in one or more of the symptoms that rendered the patient eligible for the invasive procedure, including a reduction in, for example:
i) the intensity or chronicity of the patient's radiating pain (e.g., radicular pain), including back, neck, leg or arm pain;
ii) the degree of the patient's impaired ability to perform activities of daily living, including moving, sitting, standing, bending, and working;
iii) the degree of the patient's neurologic impairment, muscle weakness, NR irritation, or other physical finding;

b) a reduction in the amount of a cytokine (e.g., soluble TNF) in the subject (e.g., in a location of interest);

c) an improvement in the abnormal findings previously observed on fluoroscopic or radiologic examination of the subject (e.g., an improved myelogram, MRI scan, CT scan, or other imaging exam);

d) the subject's no longer meeting the eligibility criteria in the predetermined SOE, CPG or clinical trial of the spinal device or fusion procedure;

e) accelerated recovery of the subject from the spinal device or fusion procedure, including fewer days spent in the hospital in the post-operative period;

f) an accelerated return of the subject to the activities of daily living;

g) an increased quality of life of the subject;

h) a decrease in the time to return to work for the subject;

i) a decrease in the time to function for the subject;

j) a reduced incidence of failed procedure, as evidence by reduced eligibility for a repeat or revision spinal device or fusion procedure;

k) a reduced incidence of adjacent level disease in dynamic stabilization or artificial disk procedures;

l) a reduced incidence of failed back surgery syndrome (FBSS), in which a spinal procedure is followed by persistent or worsening symptoms;

m) a reduced incidence of ectopic calcification after artificial disk procedures;

n) a reduced incidence of distraction injury after artificial disk procedures; and

o) a reduced incidence of BMP-induced radiculitis after intervertebral fusion procedures with BMP-2.

Other improvements in therapeutic outcome are set forth in Table 2, below.

TABLE 2

| Spinal Device or Fusion | |
|---|---|
| Procedure | Improved Outcome |
| All procedures listed | Reduction in pain on VAS |
| | Reduction in ODI score |
| | Improvement in SF36 |
| | Improvement in ambulation and |
| | activities of daily living |
| Dynamic stabilization with | Reduction in pain on flexion and |
| pedicle screw based device | extension |
| | Reduced incidence of adjacent level |
| | disease |
| Dynamic stabilization with | Reduced incidence of adjacent level |
| interspinous spacer | disease |
| Artificial disk | Reduction in pain on flexion and |
| | extension |
| | Reduced incidence of adjacent level |
| | disease |
| | Reduced incidence of ectopic |
| | calcification |
| | Reduced incidence of distraction |
| | injury |
| Interbody spinal fusion | Reduced incidence of BMP-induced |
| with BMP-2 | radiculitis |
| Kyphoplasty/vertebroplasty | Restoration of vertebral height |
| Spine Surgical Procedure using | Reduced pain, disability |
| Anti-adhesion gel or barrier | Other expected outcomes matched to |
| to prevent epidural fibrosis | the specific surgical procedure |

V. Targeted Anti-Inflammatory Therapies (TATs)

Structural Classes of TATs

TATs can be biologics (such as Abs, SMIPs, soluble receptor or coligands, or fusion proteins), polypeptides, nucleic acids, or small molecules.

Antibodies

In some embodiments of the invention, the TAT comprises an Ab, Ab fragment, or other functional equivalent thereof. Abs useful in the methods of the present invention include, without limitation, monoclonal Abs (mAbs), polyclonal Abs, Ab fragments (e.g., Fab, Fab′, F(ab′)2, Fv, Fc, etc.), chimeric Abs, mini-Abs or domain Abs (dAbs), dual specific Abs, bispecific Abs, heteroconjugate Abs, single chain Abs (SCA), single chain variable region fragments (ScFv), mutants thereof, fusion proteins comprising an Ab portion or multiple Ab portions, humanized Abs, fully human Abs, and any other modified configuration of the immunoglobulin (Ig) molecule that comprises an antigen recognition site of the required specificity, including glycosylation variants of Abs, amino acid sequence variants of Abs, and covalently modified Abs. Examples of dual specific Abs could include, but are not limited to, Abs directed to the following pairs of targets: two different antigens on the TNF molecule or TNF-R1 or R2; different chains of the TNF or TNF-R1 or R2 molecules; TNF and IL-1; TNF-R1 or R2 and TNF; TNF-R1 or R2 and IL-1; any antigen on TNF or TNF-R1 or R2 and any antigen on another IC such as IL-1, -6, -12, -15, -17, -18, -23, IFNg, GM-CSF, IL-8, MCP-1 (CCL2), and similar combinations. Methods for making such Abs are well known in the art. The Abs may be murine, rat, human, or any other origin (including chimeric, humanized, or fully human Abs). In one embodiment, the Ab recognizes one or more epitopes on an IC selected from TNF, IL-1, IL-6, IL-12, IL-15, IL-17, IL-18, IL-23, IFNg, GM-CSF, IL-8 and MCP-1 (CCL2), or recognizes one or more epitopes on an IM selected from MMP-1, 2, 3, 7, 9, 13, ADAMTS-4, 5, iNOS, NO, COX-2, and PGE2.

Antibodies also include, without limitation, agonist and antagonist Abs, as appropriate. As will be appreciated by those of skill in the art, binding affinities will vary widely between Abs, generally ranging from picomolar to micromolar levels. Methods for determining the binding affinity of an Ab are well known in the art. In some embodiments, the Ab binds an IC or IM and does not significantly bind the corresponding IC or IM from another mammalian species. In other embodiments, the Ab binds human TNF and optionally TNF from one or more non-human species.

In other embodiments, the Ab comprises a modified constant region, such as a constant region that is immunologically inert, e.g., does not trigger complement mediated lysis or stimulate Ab-dependent cell mediated cytotoxicity (ADCC) (see, e.g., U.S. Pat. No. 5,500,362). In other embodiments, the constant region is modified as described, for example, in [22]; PCT Application No. PCT/GB99/01441; and/or UK Patent Application No. 9809951.8.

Antibodies (e.g., human, humanized, mouse, chimeric) that can inhibit a protein's activity may be made by using immunogens that express the full length or a partial sequence of the protein (e.g., TNF), or cells that over expresses the protein. The Abs may be made by any method known in the art. The route and schedule of immunization of the host animal are generally in keeping with established and conventional techniques for Ab stimulation and production. Techniques for producing Abs are well known in the art including, without limitation, hybridomas, CHO cells, and other production systems; methods for primatizing or humanizing Abs and Ab fragments; methods for generating “fully human” Abs and Ab fragments; chimeric Abs; phage display technology; and recombinant technologies, such as transgenic animals and plants.

The Abs may be isolated and characterized using methods well known in the art. Abs may be isolated, for example, using conventional Ig purification procedures, such as ammonium sulfate precipitation, gel electrophoresis, dialysis, chromatography, and ultrafiltration.

SMIPs

A TAT can be a Small Modular Immuno-Pharmaceuticals (SMIP). SMIPs are single-chain polypeptides that are engineered to retain full binding and activity function of a monoclonal Ab (mAb); are approximately one-third to one-half the size of conventional therapeutic mAbs; and retain Fc-mediated effector functions. Examples of SMIP TATs for use in the present methods include TRU-015 and similar SMIPs that bind TNF or other ICs and IMs (Trubion Pharmaceuticals).

Soluble Receptors and Coligands

In some embodiments, the TAT comprises a soluble receptor or soluble co-ligand. The terms “soluble receptor”, “soluble cytokine receptor” (SCR) and “immunoadhesin” are used interchangeably to refer to soluble chimeric molecules comprising the extracellular domain of a receptor, e.g., a receptor of an IC or IM and an Ig sequence, which retains the binding specificity of the receptor and is capable of binding to the e.g., IC or IM (e.g., TNF). In one embodiment, a TNF SCR comprises a fusion of a TNF receptor amino acid sequence (or a portion thereof) from a TNF extracellular domain capable of binding TNF (in some embodiments, an amino acid sequence that substantially retains the binding specificity of the TNF receptor) and an Ig sequence. In some embodiments, the TNF receptor is a human TNF receptor sequence, and the fusion is with an Ig constant domain sequence. In other embodiments, the Ig constant domain sequence is an Ig heavy chain constant domain sequence. In other embodiments, the association of two TNF receptor-Ig heavy chain fusions (e.g., via covalent linkage by disulfide bond(s)) results in a homodimeric Ig-like structure. An Ig light chain can further be associated with one or both of the TNF receptor-Ig chimeras.

An example of a commercially available soluble receptor useful in the present invention is Enbrel® (etanercept). Enbrel® consists of recombinant human TNFR-p75-Fc fusion protein. The product is made by encoding the DNA of the soluble portion of human TNFR-p75 with the Fc portion of IgG.

Dominant-Negative Mutants

In other cases, a biologic TAT can be a dominant-negative mutant, e.g., of a polypeptide. One skilled in the art can prepare dominant-negative mutants of, e.g., the TNF receptor, such that the receptor will bind the TNF, thereby acting as a “sink” to capture TNF molecules. The dominant-negative mutant, however, will not have the normal bioactivity of the TNF receptor upon binding to TNF. The dominant negative mutant can be administered in protein form or in the form of an expression vector such that the dominant negative mutant, e.g., mutant TNF receptor, is expressed in vivo. The protein or expression vector can be administered using any means known in the art, such as intra-operatively, intraperitoneally, intravenously, intramuscularly, subcutaneously, intrathecally, intraventricularly, orally, enterally, parenterally, intranasally, dermally, or by inhalation. For example, administration of expression vectors includes local or systemic administration, including injection, oral administration, particle gun or catheterized administration, and topical administration. One skilled in the art is familiar with administration of expression vectors to obtain expression of an exogenous protein in vivo. See, e.g., U.S. Pat. Nos. 6,436,908; 6,413,942; and 6,376,471.

Antisense and siRNA Molecules

In another embodiment, a TAT may be an antisense or siRNA molecule, e.g., to a designated IC or one of the defined polypeptides in its pathway(s), or to an IM. Nucleotide sequences of the designated ICs and the defined polypeptides in their pathways, and of the IMs are known and are readily available from publicly available databases. Exemplary sites of targeting include, but are not limited to, the initiation codon, the 5′ regulatory regions, the coding sequence and the 3′ untranslated region. In some embodiments, the oligonucleotides are about 10 to 100 nucleotides in length, about 15 to 50 nucleotides in length, about 18 to 25 nucleotides in length, or more. The oligonucleotides can comprise backbone modifications such as, for example, phosphorothioate linkages, and 2′-O sugar modifications well know in the art.

In some embodiments, the TAT is a direct IC-I or a direct IM-I comprising at least one antisense or siRNA molecule capable of inhibiting or reducing the expression of a designated IC polypeptide, a defined polypeptide in the designated polypeptide's pathway, or an IM. Alternately, expression and/or release and/or receptor expression can be decreased using gene knockdown, morpholino oligonucleotides, RNA inhibition oligonucleotides (RNAi), or ribozymes, or any other methods that are well-known in the art.

Small Molecules

In some embodiments, the TAT comprises at least one small molecule IC-I or IM-I. The small molecule can be administered using any means known in the art, including via inhalation, intra-operative administration, intraperitoneally, intravenously, intramuscularly, subcutaneously, intrathecally, intradiskally, peridiskally, epidurally, perispinally, intraventricularly, orally, enterally, parenterally, intranasally, or dermally. In general, when the TAT is a small molecule, it will be administered at the rate of 0.1 to 300 mg/kg of the weight of the patient divided into one to three or more doses. For example, in an adult patient of normal weight, the doses may range from about 1 mg to about 5 g per dose.

An exemplary small molecule for use as a TAT in the present methods is thalidomide, which is an inhibitor of TNF production. The term “thalidomide” refers to an anti-inflammatory agent sold under the trademark THALOMID® (Celgene), and all pharmaceutically acceptable prodrugs, salts, solvate, clathrates and derivatives thereof The term “derivative” means a compound or chemical moiety wherein the degree of saturation of at least one bond has been changed (e.g., a single bond has been changed to a double or triple bond) or wherein at least one hydrogen atom is replaced with a different atom or a chemical moiety. Examples of different atoms and chemical moieties include, but are not limited to, halogen, oxygen, nitrogen, sulfur, hydroxy, methoxy, alkyl, amine, amide, ketone, and aldehyde. Exemplary thalidomide derivatives include, without limitation, taglutimide, supidimide, compounds disclosed in WO 94/20085, 6-alkyl-2-[3′- or 4′-nitrophthalimido]-glutarimides and 6-alkyl-3-phenylglutarimides [see e.g., (23)]; and lenalidomide, a derivative of thalidomide sold under the trademark REVLIMID® (Celgene), also known as CC-5013, which is described, for example, in [24].

Other small molecules that possess TAT, particularly TNF-I, activity include, without limitation, tetracyclines (e.g., tetracycline, doxycycline, lymecycline, oxytetracycline, minocycline), chemically modified tetracyclines (e.g., dedimethylamino-tetracycline), hydroxamic acid compounds, carbocyclic acids and derivatives, lazaroids, pentoxifylline, napthopyrans, amrinone, pimobendan, vesnarinone, phosphodiesterase inhibitors, and small molecule inhibitors of kinases. Small molecule kinase inhibitors include, without limitation, small molecule inhibitors of p38MAPK, COT, MK2, PI3K, IKKa,b,g, MEKK1,2,3, IRAK1,4 and Akt kinase. See also US Pat. Publications 2006/0046961; 2006/0046960; and 2006/0253100 for examples of small molecule inhibitors for use in the present methods.

Biogenerics, Biosimilars, Follow on Biologics, and Follow-On Proteins

The TAT, including a direct TNF-I, could also be a biosimilar, biogeneric, follow-on biologics, or follow-on protein version of a currently contemplated TAT, including a direct TNF-I. For example, once the patents covering Enbrel® (etanercept) expire, other manufacturers will likely produce molecules similar or identical to etanercept, by manufacturing processes that are substantially similar or the same, or different from, those used to manufacture Enbrel®. Their objective would be to make, offer to sell, and sell therapeutics similar or identical in structure and activity to Enbrel® (etanercept). Such molecules are generally referred to as biogenerics, generic biologics, biosimilars, follow on biologics, and follow on proteins, depending on details of the molecule, the manufacturing process and the regulatory pathway. In certain instances, the new product might differ by one or a few amino acids, which might be purported to improve the manufacturing efficiency or the therapeutic efficacy. In all such instances, these molecules are viewed as substantially the same as, or the same as currently contemplated TATs, including direct TNF-Is.

Targets and Examples of TATs

TATs for use in the invention can be IC-Is or IM-Is. In inflammation, each IC has a unique profile of biological activity, often representing multiple distinct activities. These activities are mediated by interaction of the cytokines with their receptors on a variety of inflammatory and tissue cell types. The cellular effects of ICs are mediated by intracellular signaling pathways, many of which result in activation of transcription factors which in turn activate transcription of genes encoding IC, proteinacious IM, and other proteins.

IC-Is

A TAT can be an inhibitor of one of the following IC designated polypeptides or one of the defined polypeptides in their pathways, as described further herein: TNF, IL-1, IL-6, IL-12, IL-15, IL-17, IL-18, IL-23, IFNg, GM-CSF, IL-8, MCP-1 (CCL2).

TNF-Is, Including Direct TNF-Is

TNF is produced primarily by stimulated macrophages, T cells and mast cells by cleavage of Pro TNF by TNF alpha converting enzyme (TACE). TNF induces the production of IL-1, IL-6, IL-8, IL-17, GM-CSF, PGE₂ and NO from macrophages, thus placing TNF near the top of a proinflammatory cascade. TNF also induces the production of the matrix-degrading proteolytic enzymes, MMPs and ADAMTSs, from chondrocytes, fibroblasts and other cells.

The biological effects of TNF are mediated via binding of TNF to either of two receptors, TNFR1 and TNFR2. Several signaling pathways may be activated (FIG. 2). One pathway leads to NFκB activation and is mediated by signaling proteins, including TRADD, RIP, TRAF2, MEKK-3, IKKα,β,γ, IγB-α, p50, Rel A and proteasomes. An alternative pathway to NFκB activation involves PI3K, Akt and COT prior to the IKK complex. Another pathway leads to apoptosis of the cell and is mediated by TRADD, FADD and Caspase-3 and 8 and blocked by FLICE. A fourth pathway leads to AP-1 activation and involves Rac-1, MEKK-1,2, MKK3,4,6,7, JNK, p38MAPK and MK2.

The term “TNF inhibitor” or “TNF-I” refers to any molecule which can block, suppress or reduce gene expression, protein production and processing, release, and/or biological activity of TNF, its biological receptor, coreceptor, or coligand, or a defined polypeptide in the TNF pathways (FIG. 2). Thus, examples of TNF-Is include inhibitors of any of the following polypeptides: ProTNF, TNF, TNFR1 and TNFR2, caspase 3, caspase 8, FADD, NFκB, IκB-alpha, TACE, TRADD, RIP, TRAF2, MEKK3, PI3K, Akt, COT, IKKalpha, IKKbeta, IKKgamma, p50, RelA, TRAF6, FLICE, Rac-1, MEKK-1,2, MKK3,4,6,7, JNK, p38MAPK, MK2, JUN and FOS.

A TNF-I can inhibit either or both of the two receptors TNFR1 (TNF receptor type 1) and TNFR2 (TNF receptor type 2). Some TNF-Is can inhibit a cysteine aspartase protease, such as caspase 3 or caspase 8; or can inhibit FADD; or can inhibit TRAF2. Some TNF-Is can inhibit IκB, a protein which inhibits the cell survival pathway mediator protein Nuclear factor-kappa B (NFκB). Some TNF-Is may inhibit NFκB. Examples of NFκB-Is include sulfasalazine, sulindac, clonidine, helenalin, wedelolactone, pyrollidinedithiocarbamate (PDTC), IKK-2 inhibitors, IKK inhibitors, and others, e.g., those set forth in US Pat. Publication 2006/0253100. Some TNF-Is may inhibit TNF converting enzyme (TACE), a metalloproteinase that processes pro-TNF into its mature, soluble form for release. Drugs that selectively inhibit TACE, and thereby effectively block the processing and release of mature TNF, show anti-inflammatory effects and significant decreases in cytokine production in vitro and in vivo.

Preferred inhibitors for use in the present methods are direct TNF-Is. Examples of direct TNF-Is useful in the practice of the present invention include, without limitation, the marketed products etanercept (Enbrel®, Amgen), infliximab (Remicade®, Johnson and Johnson), adalimumab (Humira®, Abbott Laboratories) and certolizumab pegol (Cimzia®; peg-antiTNF alpha Ab fragment) (formerly CDP 870; UCB/Celltech, now Nektar). Examples of direct TNF-Is currently in clinical development include the fully human anti-TNF mAb CNTO-148 (golimumab, Centocor/J&J), and the anti-TNF mAb AME-527 (Applied Molecular Evolution/Eli Lilly).

Examples of direct TNF-Is currently in pre-clinical development include the fully human anti-TNF mAb ABX-10131 (Abgenix/Amgen); several Ab fragments in development by companies such as Domantis/Peptech and AbLynx; and the SMIP TRU-015 being developed by Trubion Pharmaceuticals.

Other examples of direct TNF-Is include ABX-10131; polyclonal anti-TNF Abs such as made by therapeutic human polyclonals (THP); anti-TNF polyclonal anti-serum such as that made by Genzyme; pegylated soluble TNF receptor Type I (pegsunercept/PEGs TNF-R1); Onercept (recombinant TNF binding protein (r-TBP-1)); trimerized TNF antagonist; dominant negative TNF proteins such as Xencor's dominant negative TNF-I; modified sTNR1 (Biovation); Humicade® (CDP-570); and PN0621 (mini-Abs against TNF).

IL-1 Inhibitors, Including Direct IL-1 Inhibitors

IL-1 (a term which includes both IL-1α and IL-1β forms) is produced by processing of the precursor proteins, Pro IL-1α and Pro IL-1β, in an intracellular “inflammasome” involving P2X7, NALP3, ASC and Caspase-1 (FIG. 2). The predominant circulating form of IL-1 is IL-1β, whereas IL-1α primarily remains cell-membrane associated. IL-1 binds to its receptor, IL-1R1 and that complex then binds to IL-1RAcP (accessory protein), which enables signal transduction. The biological effects of IL-1 are mediated by two pathways (FIG. 2). One pathway leads to NFkB activation and involves MyD88, TIRAP, IRAK1,4, TRAF6 and the IKK complex shared by the TNF pathway. The other pathway leads to AP-1 activation and links the MyD88/TIRAP/IRAK-1,4 complex with Rac-1 and downstream elements shared by TNF.

The term “IL-1 inhibitor” or “IL-1-I” refers to any molecule which can block, suppress or reduce gene expression, protein production and processing, release, and/or biological activity of IL-1, its biological receptor, coreceptor, or coligand, or a defined polypeptide in the IL-1 pathways shown in FIG. 2. Examples of IL-1-Is include inhibitors of any of the following polypeptides: IL-1 alpha, IL-1 beta, Pro IL-1, P2X7, NALP3, ASC, Caspase-1, IL-1R1, IL-1RAcP, IRAK1, MyD88, TIRAP, IRAK4, TRAF6, Rac-1, MEKK-1, MEKK-2, MEKK-4, MEKK-7, JNK, JUN, FOS, MK2, p38 MAP kinase, MEKK-3, MEKK-6, AP-1, IKKalpha, -beta, or -gamma; IkB-alpha, p50, Rel A and NFκB.

Examples of IL-1-I are VX740 and VX765, small molecule caspase-1 inhibitors previously in clinical development for rheumatoid arthritis (Vertex). Some IL-1-Is can inhibit p38 kinase (p38 MAP kinase). Over 100 p38 kinase inhibitors have been identified, many of which compete with ATP and are able to bind both active and inactive (phosphorylated and unphosphorylated) forms of the MAP kinase. In other cases, tyrosine-specific phosphatases can inhibit p38 MAPK by dephosphorylating the kinase at key positions. Treatment of arthritic animal models with synthetic p38 inhibitors suggests that p38 inhibition can produce protective anti-inflammatory effects in vivo. Small molecule inhibitors of p38 MAPK have demonstrated a broad range of anti-inflammatory effects mediated by changes in cytokine production. Exemplary small molecule p38 kinase inhibitors are described in US 2005/0025765.

A direct IL-1-I can be an inhibitor of an IL-1 receptor. Interleukin-1 receptor antagonist (IL-1 Ra) is a naturally occurring molecule which reduces the biologic effects of interleukin-1 by interfering with the binding of IL-1 to its receptor (IL-1R1, interleukin-1 type 1 receptor). Kineret® (Amgen) is a recombinant form of IL-1 Ra which is FDA-approved for treating rheumatoid arthritis. Another example of a direct IL-1-I is AMG108, a mAb directed to IL-1R, currently in clinical development in rheumatoid arthritis (Amgen). AMG719 (sIL-1R2, Amgen), and IL-1 Trap (Regeneron), are also all direct inhibitors of IL-1. Another example of a direct IL-1-I is ACZ885 (a fully human anti-interleukin-1beta (anti-IL-1beta) mAb) in clinical development for Muckle-Wells Syndrome (Novartis).

IL-6 Inhibitors, Including Direct IL-6 Inhibitors

The effects of IL-6 are mediated by binding of IL-6 to IL-6Rα, either in soluble or membrane-bound form. The IL-6/IL-6Rα complex then binds to gp130 in the cell membrane to initiate signaling. Key proteins involved in the IL-6 pathway are JAK1, STAT1 and STAT3. The term “IL-6 inhibitor” or “IL-6-I” refers to any molecule which can block, suppress or reduce gene expression, protein production and processing, release, and/or biological activity of IL-6, its biological receptor, coreceptor, or coligand, or a defined polypeptide in the IL-6 pathway. Defined polypeptides in the IL-6 pathway are IL6Ralpha, gp130, JAK1, STAT1, and STAT3. An example of a direct IL-6-I is the humanized anti-IL6 receptor mAb Tocilizumab (Actemra®, Chugai). Another example of a direct IL-6-I is AMG 220, an Avimer™ protein, which binds to IL-6. AMG 220 is being studied in Crohn's disease patients. Another example of a direct IL-6-I is CNTO 328 (Anti IL-6 MAb) in clinical development for refractory multiple myeloma (Centocor). Another example of a direct IL-6-I is C326, an Avimer™ protein inhibitor of IL-6, in Crohn's Disease (Avidia).

IL-8 Inhibitors, Including Direct IL-8 Inhibitors

IL-8 is a chemokine also known as CXCL8. IL-8 mediates its activities through either of two receptors, CXCR1 and CXCR2, which are also receptors for other chemokines. Key proteins involved in the IL-8 pathway are PKC, PLC, PLD, Ras, rho and PI3K. The term “IL-8 inhibitor” or “IL-8-I” refers to any molecule which can block, suppress or reduce gene expression, protein production and processing, release, and/or biological activity of IL-8, its biological receptor, coreceptor, or coligand, or a defined polypeptide in the IL-8 pathway. Defined polypeptides in the IL-8 pathway are CXCR1, CXCR2, PKC, PLC, PLD, Ras rho and PI3K. An example of a direct IL-8-I is ABX-IL8, a fully human anti-IL-8 mAb previously in clinical development for psoriasis, COPD and chronic bronchitis (Abgenix).

IL-12 Inhibitors, Including Direct IL-12 Inhibitors

IL-12 is a heterodimer comprised of IL-12p40 and IL-12p35 chains, the former also being part of the IL-23 molecule. IL-12 mediates its activities through a heterdimeric receptor comprised of IL-12β1 and IL-12Rβ2, again the former being part of the IL-23R. Key proteins involved in the IL-12 pathway include TYK2, JAK2 and STAT4. The term “IL-12 inhibitor” or “IL-12-I” refers to any molecule which can block, suppress or reduce gene expression, protein production and processing, release, and/or biological activity of IL-12, its biological receptor, coreceptor, or coligand, or a defined polypeptide in the IL-12 pathway. Defined polypeptides in the IL-12 pathway are IL-12p40, IL-12p35, IL-12Rβ1, IL-12Rβ2, TYK2, JAK2 and STAT4. An example of an IL-12-I is the small molecule STA-5326 Meslylate in clinical development to treat gut inflammation (Synta). An example of a direct IL-12-I is ABT-874, a human mAb directed against IL-12p40, in clinical development for psoriasis and other inflammatory diseases (Abbott). Another example of a direct IL-12-I is CNTO 1275 a human mAb directed against IL-12p40, in clinical development for psoriasis and other inflammatory diseases (Centocor).

IL-15 Inhibitors, Including Direct IL-15 Inhibitors

IL-15 mediates its activities by binding to a heterotrimeric receptor comprised of an IL-15Rα chain, an IL-2/15Rβ chain and the “common γ chain” γc. Key proteins involved in the IL-15 pathway include JAK1,3 and STAT3,5. The term “IL-15 inhibitor” or “IL-15-I” refers to any molecule which can block, suppress or reduce gene expression, protein production and processing, release, and/or biological activity of IL-15, its biological receptor, coreceptor, or coligand, or a defined polypeptide in the IL-15 pathway. Defined polypeptides involved in the IL-15 pathway are IL-15Ralpha, IL-2/IL-15Rbeta, the common gamma chain “gamma-c”, JAK1, JAK3, STAT3 and STAT5. An example of a direct IL-15-I is AMG 714, a fully human mAb (formerly called HuMAX15) directed against IL-15 in clinical development by Amgen/Genmab.

IL-17 Inhibitors, Including Direct IL-17 Inhibitors

IL-17 mediates its effects via an IL-17R that is expressed on virtually all cell types. Key proteins involved in the IL-17 pathway include TRAF6 and the same downstream IKK complex leading to NFκB activation as in IL-1 pathway. The term “IL-17 inhibitor” or “IL-17-I” refers to any molecule which can block, suppress or reduce gene expression, protein production and processing, release, and/or biological activity of IL-17, its biological receptor, coreceptor, or coligand, or a defined polypeptide in the IL-17 pathway. Defined polypeptides in the IL-17 pathway are IL-17R, MyD88, TIRAP, IRAK1, IRAK4, TRAF6, IKKalpha, IKKbeta, IKKgamma, IkappaB-alpha, p50, Rel A, Proteasome, NFκB and FLICE.

IL-18 Inhibitors, Including Direct IL-18 Inhibitors

IL-18 binds to a 4-chain receptor complex comprised of IL-18Rα, IL-18Rβ, IL-1RAcP and a pathway chain. A naturally-occurring antagonist of IL-18 called IL-18BP blocks the binding of IL-18 to its receptor. Key proteins involved in the IL-18 pathway include MyD88 and all the downstream elements via TRAF6 leading to NFκB activation as in IL-1 pathway. The term “IL-18 inhibitor” or “IL-18-I” refers to any molecule which can block, suppress or reduce gene expression, protein production and processing, release, and/or biological activity of IL-18, its biological receptor, coreceptor, or coligand, or a defined polypeptide in the IL-18 pathway. Defined polypeptides in the IL-18 pathway are Pro IL-18, P2X7, NALP3, ASC, Caspase-1, IL-18, IL-18Ralpha, IL-18Rbeta, IL-1RAcP, IL-18R signaling chain, IL-18BP, MyD88, TIRAP, IRAK1, IRAK4, TRAF6, IKKalpha, IKKbeta, IKKgamma, IkappaB-alpha, p50, Rel A, Proteasome, NFκB, FLICE, Rac-1, MEKK-1, MEKK-2, MKK3, MKK4, MKK6, MKK7, JNK, p38MAPK, MK2, JUN, FOS and AP-1.

IL-23 Inhibitors, Including Direct IL-23 Inhibitors

IL-23 is a heterodimer of IL-12p40 and IL-23p 19 chains and binds to a heterodimeric IL-23 receptor comprised of IL-12Rβ1 and IL-23R. Key proteins involved in the IL-23 pathway include TYK2, JAK2 and STAT3. The term “IL-23 inhibitor” or “IL-23-I” refers to any molecule which can block, suppress or reduce gene expression, protein production and processing, release, and/or biological activity of IL-23, its biological receptor, coreceptor, or coligand, or a defined polypeptide in the IL-23 pathway. Defined polypeptides in the IL-23 pathway are IL-12p40, IL-23p19, IL-12Rβ1, IL-23R, TYK2, JAK2 and STAT3. An example of a direct IL-23-I is ABT-874, a human mAb directed against IL-12p40, in clinical development for psoriasis and other inflammatory diseases (Abbott). Another example of a direct IL-23-I is CNTO 1275, a human mAb directed against IL-12p40, in clinical development for psoriasis and other inflammatory diseases (Centocor).

IFNγ Inhibitors, Including Direct IFNγ Inhibitors

The effects of IFNγ are mediated by homodimers of IFNγ binding to a receptor comprised of an IFNγRα ligand-binding chain and an IFNγRβ signaling chain. Key proteins involved in the IFNγ pathway include JAK1, JAK2 and STAT1. The term “IFNγ inhibitor” or “IFNγ-I” refers to any molecule which can block, suppress or reduce gene expression, protein production and processing, release, and/or biological activity of IFNγ, its biological receptor, coreceptor, or coligand, or a defined polypeptide in the IFNγ pathway. Defined polypeptides in the IFNγ pathway are IFNγRα, IFNγRβ, JAK1, JAK2 and STAT3.

GM-CSF Inhibitors, Including Direct GM-CSF Inhibitors

GM-CSF binds to a heterodimeric receptor comprised of GMRα and a common β subunit, βc. Key proteins involved in the GM-CSF pathway include JAK2, STAT5, SHP-2, RAS and Raf-1. The term “GM-CSF inhibitor” or “GM-CSF-I” refers to any molecule which can block, suppress or reduce gene expression, protein production and processing, release, and/or biological activity of GM-CSF, its biological receptor, coreceptor, or coligand, or a defined polypeptide in the GM-CSF pathway. Defined polypeptides in the GM-CSF pathway are GMRalpha/Beta-c, JAK2, STAT5, SHP-2, RAS and Raf-1.

MCP-1 Inhibitors, Including Direct MCP-1 Inhibitors

MCP-1 is a chemokine also known as CCL2. MCP-1 mediates its activities by binding to a single receptor, CCR2. Key proteins involved in the MCP-1 pathway include PKC and the same IKK complex and downstream elements as in TNF/IL-1 pathway leading to NFkB activation. The term “MCP-1 inhibitor” or “MCP-1-I” refers to any molecule which can block, suppress or reduce gene expression, protein production and processing, release, and/or biological activity of MCP-1, its biological receptor, coreceptor, or coligand, or a defined polypeptide in the MCP-1 pathway. Defined polypeptides in the MCP-1 pathway are CCR2, PKC, IKKalpha, IKKbeta, IKKgamma, IkappaB-alpha, p50, Rel A, Proteasome, NFκB and FLICE. An example of a direct MCP-11 is ID9, a mAb directed against the MCP-1 receptor CCR2 (Millenium).

IM-Is

A TAT can be an inhibitor of one of the following IMs: MMP-1,2,3,7,9,13; ADAMTS-4, 5; iNOS, NO, COX-2, and PGE2.

MMP Inhibitors, Including Direct MMP Inhibitors

The term “MMP-1, 2, 3, 7, 9, 13 inhibitor” or “MMP-1-I, 2-I, 3-I, 7-I, 9-I, 13-I” refers to any molecule which can block, suppress or reduce gene expression, protein production and processing, release, and/or biological activity of the respective MMP-1, 2, 3, 7, 9, or 13 polypeptide, or the biological receptor, coreceptor, or coligand of the same. Examples of broad-spectrum (nonspecific) direct MMP-Is include the small molecule compounds marimastat and batimastat, previously in clinical development (British Biotech, Inc).

An example of a class of direct MMP-13-I with selectivity relative to other MMPs is the small molecule genus of 3-hydroxy-4-arylsulfonyltetrahydropyranyl-3-hydroxamic acids previously in clinical development (Pfizer).

An example of a direct MMP-2-I and direct MMP-9-I is XL784, a relatively selective small molecule compound in clinical development (Exelixis).

iNOS Inhibitors, Including Direct iNOS Inhibitors

The term “iNOS inhibitor” or “iNOS-I” refers to any molecule which can block, suppress or reduce gene expression, protein production and processing, release, and/or biological activity of iNOS, or its biological receptor, coreceptor, or coligand. An example of a direct iNOS-I is GW274150, a small molecule compound in clinical development for rheumatoid arthritis and migraine (GSK). Another example of a direct iNOS-I is aminoguanidine, a small molecule compound evaluated in clinical endotoxemia (Radboud University). Another example of a direct iNOS-I is SC-51, a small molecule compound in clinical development for asthma (Pfizer).

COX-2 Inhibitors, Including Direct COX-2 Inhibitors

The term “COX-2 inhibitor” or “COX-2-I” refers to any molecule which can block, suppress or reduce gene expression, protein production and processing, release, and/or biological activity of COX-2, or its biological receptor, coreceptor, or coligand. Examples of direct COX-2-I are celecoxib (Celebrex®, Pfizer) and rofecoxib (Vioxx®, Merck), small molecule compounds for treatment of inflammation and pain.

Combination Therapies

Multiple TAT Inhibitors, Including Multiple TNF-I

The present disclosure also contemplates the use of multiple TATs in the methods described herein. The combination of different TATs that have specificity for different points in a pathway, e.g., a TNF pathway, or different points in two or more different pathways, may be more efficient than the use of a single TAT. For instance, TNF itself may be inhibited at multiple points and by targeting various mechanisms in the TNF pathways. Potential inhibition points include TNF transcriptional synthesis, translation, or shedding mediated by MMPs. TNF and other similar bioactive substances are first produced in an inactive form and transported to the cell membrane. Upon activation, the active part of the pro-TNF is cleaved and released. This process is called shedding and may be initiated by one or more MPs. TNF may also be inhibited after its release, either by Abs (e.g., by infliximab, adalimumab, or CDP-870) or soluble receptors (e.g. etanercept).

The combination of two or more drugs that act through different mechanisms may therefore induce a more efficient inhibition of an IC or IM pathway than the use of one single drug. In one embodiment, a direct TNF-I is used in combination with a second direct TNF-I, or with a non-specific TNF-I or an inhibitor of a different IC or IM. In another embodiment, a direct TNF-I is used in combination with an NFκB inhibitor such as sulfasalazine, sulindac, clonidine, helenalin, wedelolactone, pyrollidinedithiocarbamate (PDTC), IKK-2 inhibitors, IKK inhibitors, and others, e.g., those set forth in US Pat. Publication 2006/0253100.

Combinations of Devices and TATs

Combination devices comprising a TAT and a device described herein are also contemplated. The combination of the device and TAT can be any kind of physical combination, e.g., a coating comprising the TAT on or in the device; a depot formulation or reservoir capable of delivering the TAT on, adjacent to, or in immediate environment of the device; a hydrogel, polymeric, or controlled-release formulation comprising the TAT on or in the device, to deliver the TAT on, adjacent to, or in the immediate environment of the device; a sustained release formulation comprising the TAT on or in the device, wherein the release is delayed temporarily after implantation of said device, to allow for TAT delivery weeks or months post implantation on, adjacent to, or in the immediate environment of the device.

For example, an annular repair or replacement device could include a coating of a TAT on the surfaces of the device, or provide a controlled release formulation in the body of the device. With mesh based annular repair devices, the TAT could be formulated in controlled release microsphere formulation embedded in the mesh biomaterial.

Dynamic stabilization devices or interspinous process spacers could include a coating of a TAT on the surfaces of the device (the pedicle screws, posterior fixation elements, and flexible connecting rods and structures), or as a device-based depot formulation (using the device components above) for delivery of the TAT locally.

Spine fusion devices, including but not limited to cages, machined allograft, plates, screws, rods, vertebral body replacements, and interbody spacers, could include a coating of a TAT on the surfaces of the device, or as a device-based depot formulation (using the device components above) for delivery of the TAT locally. For BMP fusion devices, TAT could be co-formulated with BMP or other bone growth stimulatory proteins.

Kyphoplasty/vertebroplasty/vertebral restoration devices could include an initial dosing of a TAT prior to the injection of the device, or the TAT could be formulated in the body of the device to be retained when the flowable phase hardens in situ. In other embodiments, depot formulations of TAT could be suspended in the flowable phase of the device, to be distributed throughout the vertebral body as the flowable phase hardens in situ.

Facet replacement and fixation devices, and dural repair devices could include a coating of a TAT on the surfaces of the device, or as a device-based depot formulation for delivery of the TAT locally.

Supplemental Active Ingredients

A TAT, e.g., TNF-I, may be administered in combination with other drugs or compounds, provided that these other drugs or compounds do not significantly reduce or eliminate the desired results according to the present invention, e.g., the effect on a IC or IM of interest such as TNF. Specific methods of the invention comprise administering a TAT in combination with an SAI. The SAI can be any TAT. Further, the SAI can be any therapeutic agent capable, for example, of relieving pain, providing a sedative effect or an antineuralgic effect, or ensuring patient comfort. Examples of the SAIs include, but are not limited to, opioid analgesics, non-narcotic analgesics, anti-inflammatories, cox-2 inhibitors, α-adrenergic receptor agonists or antagonists, ketamine, anesthetic agents, NMDA antagonists, immunomodulatory agents, immunosuppressive agents, antidepressants, anticonvulsants, antihypertensives, anxiolytics, calcium channel blockers, muscle relaxants, corticosteroids, hyperbaric oxygen, neuroprotectants, antibiotics, other therapeutics known to relieve pain, and pharmaceutically acceptable salts, solvates, hydrates, stereoisomers, clathrates, prodrugs and pharmacologically active metabolites of any of the foregoing therapeutic agents.

In another embodiment, the supplement active ingredient is a non-steroidal anti-inflammatory drug (NSAID), corticosteroid, slow acting antirheumatic drug (SAIRD), disease modifying antirheumatic drug (DMARD), short-acting LA, or long-acting LA. In yet another embodiment, the SAI is a propionic acid derivative, such as ibuprofen or naproxen. Structurally related propionic acid derivatives having similar analgesic and anti-inflammatory properties are also intended to be encompassed by this group. In another embodiment, the SAI is an acetic acid derivative, for example alclofenac, diclofenac sodium, or sulindac. Structurally related acetic acid derivatives having similar analgesic and anti-inflammatory properties are also intended to be encompassed by this group. The SAI may also be a fenamic acid derivative such as, without limitation, enfenamic acid, etofenamate, or flufenamic acid. Structurally related fenamic acid derivatives having similar analgesic and anti-inflammatory properties are also intended to be encompassed by this group.

In other embodiments, the SAI is a carboxylic acid derivative, a butyric acid derivative, or oxicam, a pyrazole, or a pyrazolon.

In another embodiment, the SAI is an antibiotic. Exemplary antibiotics include, without limitation, sulfa drugs (e.g., sulfanilamide), folic acid analogs (e.g., trimethoprim), beta-lactams (e.g., penacillin, cephalosporins), aminoglycosides (e.g., stretomycin, kanamycin, neomycin, gentamycin), tetracyclines (e.g., chlorotetracycline, oxytetracycline, and doxycycline), macrolides (e.g., erythromycin, azithromycin, and clarithromycin), lincosamides (e.g., clindamycin), streptogramins (e.g., quinupristin and dalfopristin), fluoroquinolones (e.g., ciprofloxacin, levofloxacin, and moxifloxacin), polypeptides (e.g., polymixins), rifampin, mupirocin, cycloserine, aminocyclitol (e.g., spectinomycin), glycopeptides (e.g., vancomycin), and oxazolidinones (e.g., linezolid).

In another embodiment, the SAI is capable of providing a neuroprotective effect. In addition to TNF, other examples of neuroprotective agents include, without limitation, erythropoietin (Epo), Epo derivatives or mimetics, and other compounds that stabilize or protect neurons from injury. Epo and its derivatives or mimetics might offer particular advantages, or otherwise be particularly appropriate, to patients undergoing surgery. Usage of Epo or Epo-mimetics as neuroprotectants may be limited by the difficulty in separating the neuroprotective effects of Epo from the erythrogenic effects. However, a particular setting in which such erythrogenic “side effects” are acceptable is in patients about to undergo surgery, in whom a moderate and temporary increase in hematocrit may be desirable. Thus, in peri-operative usage to improve surgical outcome, Epo may offer surprising advantages as a neuroprotectant.

The SAI could also be ozone as delivered to the spinal structure by ozone therapy [25].

VI. Administration Regimens

Any route of administration for a TAT and any type of formulation of a TAT can be used in the present methods. Routes of administration for currently approved TATs, such as TNF-Is, are known to those of ordinary skill in the art, consisting primarily of systemic injection, e.g., intramuscular injection, SC injection, or IV infusion. [See, e.g., (26)]. Other more invasive routes of administration, however, are also specifically contemplated in the present methods, e.g., including intrathecal, intradiskal, and epidural routes. Thus, a TAT can be administered using any of the following routes of administration: intra-operatively, intravenously, intramuscularly, SC, intrathecally, intradiskally, peridiskally, epidurally, perispinally, orally, enterally, parenterally, intranasally, dermally (e.g., transdermally), or by inhalation.

A TAT composition can be administered to a site, e.g., a site of a spinal device or fusion procedure, using any suitable method, such as delivery through a needle or other cannulated device (see, e.g., *U.S. Pat. Nos. 6,375,659, 6,348,055 and 6,582,439). The TAT composition may be delivered via a single injection, or by multiple injections at or near the surgical site. A suitable volume of a TAT composition can be determined using methods well known in the art, for example by adding barium, tungsten, or other substances to render the material radiopaque.

In preferred embodiments of the present invention, a pump is used to deliver one or more TATs and optionally other therapeutic agents continuously over an extended period of time, or intermittently at distinct times of administration. These pump devices preferably comprise a pump; a reservoir coupled to the pump; and a catheter operably coupled to the pump and configured to deliver the therapeutic agent to the target site. For purposes of allowing ease of treatment over an extended period of time, the catheter may be designed such that it is removable from the pump, and may be capped and retained within the patient's body such that repeated doses may be administered through the catheter without the need for repeatedly inserting and removing the catheter. The timing and dosage regimen may be pre-set, may be monitored and adjusted by computer, or may be monitored and adjusted by the patient or a treating care worker to provide the appropriate dosage at the right time. Use of such pump and catheter systems is particularly advantageous for allowing administration of the maintenance dosage regimen of TATs in accordance with the present invention. The catheter may be implanted at the time of a spinal device or fusion procedure, such as a diskectomy, such that subsequent dosage and targeting of TATs to the particularly affected areas may be accomplished without further surgical intervention. A pump can be an infusion pump, an osmotic pump, or an interbody pump.

In some embodiments, a controlled release formulation, e.g., a depot, is used to deliver one or more TATs. A controlled release formulation can include, without limitation, a capsule, microsphere, particle, gel, wafer, pill, etc. A controlled release formulation can exhibit a controlled release rate of the one or more TATs, e.g., over a period from about 12 hours to about 3 months, or any time therebetween, e.g., 1 day to 1 week; 1 day to 1 month; 1 day to 2 months; etc. A controlled release formulation can include one or more biopolymers known to those having ordinary skill in the art, e.g., poly(alpha-hydroxy) acids, poly(lactide-co-glycolide) (PLGA), polylactide (PLA), polyglycolide (PG), PEG, PEG derivatives, PEG conjugates, polyvinyl alcohol (PVA), polyurethane esters, polycarbonates, copolymers, and others, including those as set forth in US 2006/0046961.

Administration of a TAT can be local and/or targeted or non-local; through more invasive or less invasive means; and at any suitable dose, e.g., as determined by a healthcare service provider.

Local and/or Targeted Administration

As described herein, the methods can utilize local or targeted administration of a TAT. Although these methods of administration can be moderately invasive, they are less invasive than a surgical procedure, and the local and/or directed administration of the TAT may offer the best way to selectively address the particular injury to the spine, disk, or surrounding nerves. For example, the induction regimens of the present invention can involve locally directed administration of one or more TATs to allow effective interruption of the inflammatory pathways, e.g., the TNF pathway, and to alleviate neuropathic pain. Local administration may also reduce unwanted systemic side effects of the TAT, by permitting the use of lower doses, or by limiting systemic exposure through local delivery. In some embodiments, local administration can mean placement of the delivery vehicle within 10 cm (e.g., within 10, 9, 8, 7, 6, 5, 4, 3, 2, 1, or 0.5 cm) of the site of the presumed injury, into an appropriate location in a suitable form. In some cases, local administration permits the delivery of higher TAT concentrations than could be achieved systemically.

Intrathecal Delivery

One means of local delivery is the use of an intrathecal delivery system. Intrathecal delivery systems frequently comprise an infusion pump and an intraspinal catheter. One or more TATs may thus be delivered to the spinal canal or intrathecal space. Both the pump and the catheter may be implanted. In such cases, the pump is usually programmable, such that chronic infusion of one or more TATs may be accomplished over a period of time, and the pump or a reservoir may be periodically refilled. Alternatively, the pump may be external, and used for the delivery of one or more TATs. In such cases, administration may be controlled manually, or a programmable pump may still be used. Intrathecal delivery may be the preferred means of systemic delivery because the drug does not enter the bloodstream, and thus will not cross the blood-brain barrier into the brain.

Intrathecal or Epidural Pump and Catheter Systems

In the present invention, one or more TATs may be administered by means of an intrathecal or epidural delivery device. Such a delivery device can be any one of the currently marketed Medtronic Sofamor Danek intrathecal drug delivery devices, including but not limited to any of the SynchroMed® EL models, any of the SynchroMed® II models, or the MiniMed Paradigm®-REAL-TIME Insulin Pump and Sertable™ infusion sets, described above, and/or their successors. These pumps may be implanted to prevent or postpone the need for a spinal device or fusion procedure, and/or to improve the outcome of the spinal device or fusion procedure, e.g., to treat surgery-induced injury and pain using one or more TATs. Additionally, one or more TATs may be used to coat the intrathecal catheter prior to implantation. One or more TATs may also be used in the patient screening test in order to assess the effectiveness of the drug prior to implantation of an intrathecal delivery device.

A number of infusion pumps are currently marketed by Medtronic Sofamor Danek, known collectively as the SynchroMed® Infusion system. The Medtronic SynchroMed® Programmable Drug Infusion Pump is a fully implantable, programmable, battery-powered device that stores and delivers medication according to instructions received from the programmer.

Intrathecal drug delivery provides a treatment option that is fully reversible, i.e., the system can be turned off or fully removed with little or no consequence. A further benefit of this system is that in most patients, pain is alleviated using a lower dose of medication than is required to achieve the same effect via oral or IV routes because the pain medication is delivered directly to the appropriate (e.g., intrathecal or epidural) space. For example, pain relief can be achieved using intrathecal drug delivery with a dose that is 99.9967% lower compared to the dose required to achieve the same result orally [27]. This also reduces the side effects that may be associated with higher doses of the medication, such as nausea, vomiting, sedation, and constipation, thus improving the patient's quality of life. Unlike long-term IV or epidural therapy, intrathecal drug delivery also allows a patient to tailor his medication to his lifestyle. Under the guidance of their clinician, patients can administer themselves an additional dose of medication, known as a “bolus” dose, if they feel a spike in pain or in preparation for an activity that is expected to result in a spike in pain. Finally, as the system is fully implanted, there is a low risk of infection.

Epidural, Intradiskal, and Peridiskal Administration

Administration using an epidural syringe is a well-known method of administering therapeutic agents, such as anesthetics or steroids, to the spine. Using fluoroscopic or other means to guide the epidural syringe to the desired location, therapeutics may be delivered to the area known as the epidural space, which is adjacent to the dura mater and within the spinal canal formed by the surrounding vertebrae. By administering TATs using an epidural syringe, a single dose may be targeted directly to the area of insult or injury near the spine. Alternatively, use of an epidural catheter and pump system allows for an extended dosage regimen of repeat dosings to the epidural space.

A particularly useful means for administering TATs for an induction regimen as described herein comprises intradiskal administration. In preferred embodiments, intradiskal administration is accomplished using devices such as intradiskal or epidural syringes and other spinal injections, optionally combined with fluoroscopic guidance to provide means for conducting diskography for targeting TATs to the damaged disk or disks. In one particular embodiment, prior to or subsequent to intradiskal injection of one or more TATs, one or more TATs may additionally be administered targeted to the area just adjacent to the disk (the peridiskal area) and/or epidurally. Thus, in certain preferred embodiments of the invention, a single epidural syringe, or other means of spinal injections, may be used to administer one or more TATs, with or without other active agents, such as an LA, steroids, or other treatment, both intradiskally and peridiskally and/or epidurally (“intradiskal/peridiskal administration” or “intradiskal/epidural administration” or “intradiskal/peridiskal/epidural administration”). In one embodiment, the syringe may have two compartments, each containing a dose of at least one TAT intended for its respective targeted area. In another embodiment, the surgeon administering the TATs can manipulate the syringe in a manner such that part of the dosage is injected intradiskally, for example, by depressing the syringe lever only partway, thereby administering an intradiskal dosage; and retaining a peridiskal/epidural dosage to remain in the syringe; while the syringe is being withdrawn from the disk, the surgeon can administer the peridiskal/epidural dosage to the peridiskal/epidural region by pausing while the needle is adjacent to, but outside of the affected disk, and depressing the syringe further to administer the peridiskal/epidural dosage. Analogously, the surgeon can pause during insertion of the syringe and, while the needle is located adjacent to, but has not yet pierced, the affected disk, depress the syringe lever partway in order to direct a peridiskal/epidural dosage to the peridiskal/epidural area. Following such administration, the syringe can then be guided into the disk, and an intradiskal dosage administered.

Intradiskal administration can also be combined with other therapies, such as IDET, or with a diagnostic apparatus, such as the pump used for functional anesthetic diskograpy owned by Kyphon.

Other Means of Local and Targeted Administration

Other means of local administration include PR infiltration under fluoroscopic guidance, implants which are coated with a substance comprising one or more TATs, or biomaterials which comprise one or more TATs, and which are designed for the controlled delivery of TATs, including bioresorbable materials, e.g., controlled release formulations as described above which will release the TATs as they are resorbed into the body. Suitable resorbable materials are well known to those having ordinary skill in the art.

Systemic, Non-Local, and/or Non-Targeted Administration

In addition to local or targeted administration, the methods and materials of the present invention may also utilize systemic administration of one or more TATs. Unlike local or targeted administration, systemic administration tends to be less invasive, is typically “non-local” to the site of injury, and, importantly, may be performed as an out-patient treatment, or may even be self-administered by the patient. Thus, the systemic means of administration are advantageous in that they are less disruptive to the patient's life, and therefore, may result in improved compliance by patients with the prescribed regimens.

Systemic administration of one or more TATs can be used in any regimen, and is frequently used for the maintenance regimen in an induction-maintenance regimen as described herein. The maintenance regimens may provide for long-term relief of back pain or neuropathic pain by administering one or more TATs to allow the continued inhibition of the inflammatory pathway(s).

Parenteral Administration

Parenteral administration includes various methods of infusion or injection of the drug. Preferred methods of parenteral administration may include IV injection or infusion directly into the bloodstream. Other methods of parenteral administration include intramuscular; SC; transdermal; and intraperitoneal administration.

Other Means of Systemic Delivery

Other means of systemic delivery may include the following delivery routes: oral, that is, ingested as a tablet, capsule or fluid; inhalation or intranasal; transmucosal or buccal; or transdermal, such as through use of a skin patch. Suspensions or solutions for intramuscular injections may contain together with the active compound, a pharmaceutically acceptable carrier, such as e.g., sterile water, olive oil (or other vegetable or nut derived oil), ethyl oleate, glycols, e.g., propylene glycol, and if so desired, a suitable amount of lidocaine hydrochloride. Adjuvants for triggering the injection effect can be added as well. Solutions for IV injection or infusion may contain as carrier, e.g., sterile water, or preferably, a sterile isotonic saline solution, as well as adjuvants used in the field of injection of active compounds. Such solutions would also be suitable for i.m. and i.c.v. injection.

Induction and Maintenance Regimens

In particular embodiments, the present methods can include the use of a novel regimen comprising an induction regimen followed by a maintenance regimen for administration of one or more TATs. For example, the methods may comprise administering to the subject an induction regimen comprising a therapeutically effective amount of a TAT (e.g., a TNF-I); and administering to the subject a maintenance regimen comprising a therapeutically effective amount of the same or a different TAT. An induction regimen and a maintenance regimen can independently include multiple administrations of a TAT (e.g., 2, 3, 4, 5, 6, 8, 10, or more separate administrations). In some embodiments, a maintenance regimen will comprise more separate administrations of a TAT than an induction regimen. For example, an induction regimen may comprise one administration of a TAT (e.g., a single intradiskal administration), while a maintenance regimen may comprise weekly or monthly intramuscular injections for a period of 1 month, 2 months, 3 months, 6 months to a year, or longer.

The induction regimen can provide for a substantive, rapid, or clinically relevant induction of protection from neuronal insult or remission of pain or other symptoms (e.g., weakness, numbness). Although not being bound by theory, it is believed that the induction regimen can provide for interruption of one or more of the biological and physiological processes which contribute to symptoms such as severe and/or persistent pain, and/or injury, mediated by inflammatory cytokines or mediators. The induction regimen may comprise administering at least one dose (an “induction dose” or “loading dose”) of at least one TAT, e.g., a TNF-I, such that induction of remission of pain or other symptoms, or protection from exacerbation of symptoms occurs.

An induction regimen can involve a more invasive route of administration than a maintenance regimen. A more invasive route of administration can be evaluated according to the invasiveness spectrum defined previously. Thus, an induction regimen can include a mode of administration selected from intrathecal, intradiskal, epidural (including transforaminal and periradicular), or perispinal, while a maintenance regimen can be selected from perispinal (provided the induction regimen is not perispinal), IV, intramuscular, or SC administration. In some cases, an induction regimen will be selected from intradiskal or epidural, while a maintenance regimen will be selected from IV, intramuscular, or SC administration.

An induction regimen can involve a more local or targeted administration than a maintenance regimen. A more local administration can be obtained targeting the administration to the site of injury or in close proximity to the site of injury in the subject. Modes of administration that result in “systemic” administration are understood by those having ordinary skill in the art to be “non-local” and non-targeted. Thus, in some cases, an induction regimen will include administration in proximity to the site of spinal pathology (e.g., site of an HD, SS, adhesion, sensory nerve, or internal disk derangement), while the maintenance regimen will involve non-targeted administration. For example, an induction regimen can involve intradiskal or epidural administration to an HD, site of SS, adhesion, or internal disk derangement, while a maintenance regimen can involve systemic administration, e.g., through IV, intramuscular, or SC administration.

In preferred embodiments, the more local and/or more invasive route of administration of an induction regimen results in a higher concentration of drug in or at the presumed site of therapeutic action or pathology, such as the affected nerve root.

An induction regimen comprises a lower dose per administration of a TAT than a maintenance regimen. The dose per administration can be evaluated by those having ordinary skill in the art. Typically, the lower dose per administration of an induction regimen is less than about 50% of the maintenance dose per administration, e.g., less than about 50%, 45%, 40%, 35%, 30%, 25%, 20%, 15%, 10%, or 5% of the maintenance dose per administration.

In particular embodiments, an induction regimen may comprise local (e.g., at the site of an HD), invasive administration (e.g., epidural, intradiskal, peridiskal administration) of one or more low doses per administration (low as compared to the maintenance dose per administration) of at least one TAT, e.g., in an amount sufficient to provide clinically meaningful relief of pain or other symptoms. In preferred embodiments, an “induction regimen” comprises one to seven (e.g., 1, 2, 3, 4, 5, 6, or 7) intradiskal or epidural (including periradicular and transforaminal) administrations of at least one TNF-I selected from the group consisting of Enbrel® (etanercept); Humira® (adalimumab); Humicade® (CDP-570); Cimzia® (certolizumab pegol); Remicade® (infliximab), CNTO-148, Peptech antibody, Wyeth-Trubion SMIP, Wyeth-Ablynx antibody fragement, and PN0621 (mini-antibodies against TNF).

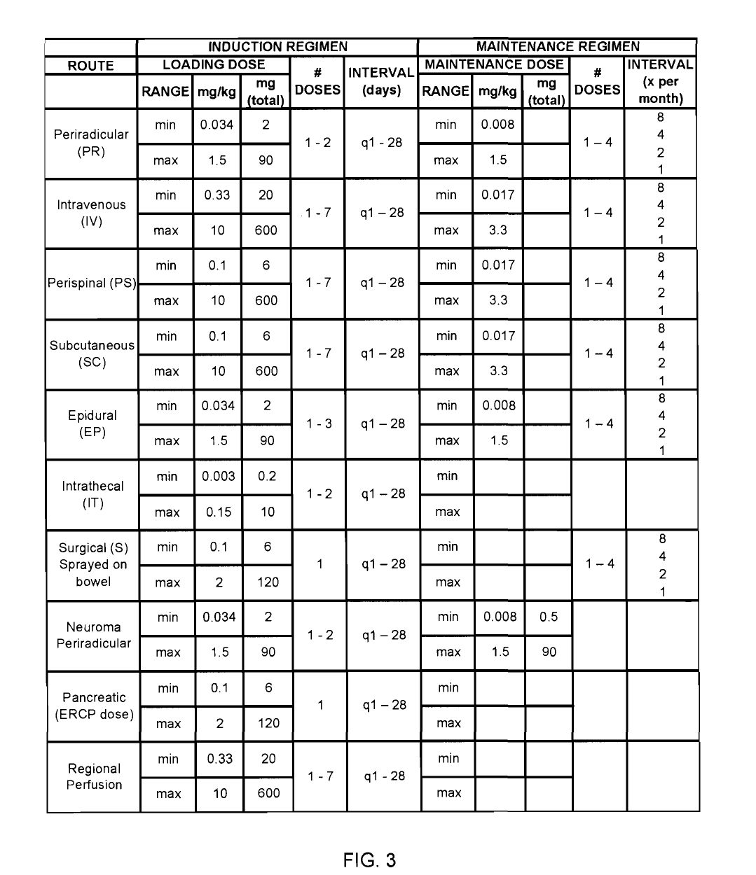

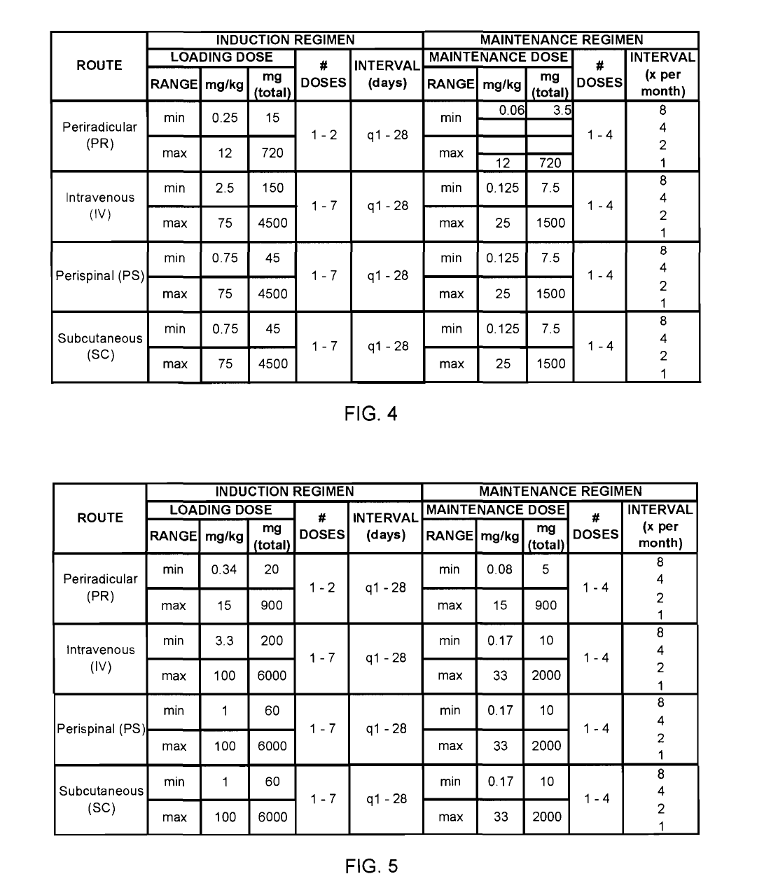

Preferred dosage ranges for an “induction regimen” of a TAT will vary depending upon clinical factors observed by the clinician, the indication, and the particular TAT, and will generally comprise administration of a “loading dose” of at least one TAT, or a dose which will generally achieve clinically meaningful induction of protection from neuronal insult or relief of pain upon administration. In preferred embodiments, the induction regimen will provide protection from injury or relief of pain or other symptoms within several hours of administration. In some embodiments, the induction regimen comprises administration of a “loading dose” of at least one TAT (e.g., TNF-I) via local administration, for example via epidural, intradiskal, intradiskal/peridiskal, intradiskal/epidural or intrathecal administration. Preferred induction regimens for several approved TNF-Is are provided in FIGS. 3-5.

A maintenance regimen can provide for durable protection from neuronal insult or relief from pain or other symptoms similar to the relief afforded by an induction regimen. A maintenance regimen can comprise administration of at least one dose of at least one TAT to maintain such relief for a period of time (e.g., a “maintenance dose”), preferably the period of time being at least one to twenty-four hours, at least twenty-four hours to one week, or at least one week to three months. A maintenance regimen may accompany and/or follow administration of an induction regimen.

A maintenance regimen of a TAT will also vary depending upon clinical factors observed by the clinician, the indication, and the type of inhibitor, and can comprise administration of a “maintenance dose” of at least one TAT (e.g., TNF-I), or a dose which will generally achieve durable induction of relief from pain or protection from exacerbation of symptoms when administered concurrently with and/or subsequent to, administration of an “induction regimen.” A “maintenance regimen” of a TAT may be administered once, or may be administered periodically (e.g., hourly, every 4 hours, every 6 hours, every 12 hours, daily, weekly, monthly, bimonthly) according to a dosage regimen prescribed by the treating physician. The maintenance regimen comprises administration of a maintenance dose of at least one TAT via a less invasive or less local mode of administration than an induction regimen but that is still effective for durable induction of protection from neuronal insult or relief from pain. For example, a maintenance dose of TAT will be administered via less invasive modes of administration, such as IV, intramuscular, or SC administration. In some embodiments, the maintenance regimen comprises administration of at least one maintenance dose via continuous dosage means, such as a pump and catheter. The catheter may be inserted during the course of administering the induction regimen, or may be separately inserted. Preferred maintenance regimens for several approved TNF-Is are provided in FIGS. 3-5.

Routes of administration, timing of administration, and choice of TAT for the “induction regimen” and “maintenance regimen” will vary depending upon the practitioner's choice of regimen, the indication, and the type of inhibitor. The criteria that might lead a skilled practitioner to choose a particular TAT for a particular regimen will often include drug concentration, lipophilicity, solubility, half life, formulation characteristics, pH, pKa, known adverse events profile, tmax, potency, and affinity (e.g., for the target), among other factors. The relative weight and strength of the applicability of each of these criteria would depend, in part, on the indication and on the site of administration. Thus, for example, since a limited volume of agent can be safely injected intradiskally, an agent high in concentration might be chosen to maximize the dosage given. In an epidural route of administration, a lipophilic agent might limit spread of the TAT to distant, non-pathologic locations within the epidural space, while choice of a large protein TAT or a depot formulation might limit migration out of the epidural space. Moreover, in certain embodiments, the induction regimen is administered and completed prior to beginning administration of the maintenance regimen. In others, the maintenance regimen may begin at or near the same time as the induction regimen.

The TAT for use in the maintenance regimen may be the same as or different than the TAT for use in the induction regimen. The formulation of the TATs can be the same or different, e.g., both can be an aqueous formulation, or one could be aqueous while the other is an oil-in-water emulsion, or one could be aqueous while the other could be a depot or controlled-release formulation.

In an embodiment, the induction regimen and/or maintenance regimen may be administered by means of a catheter and pump system, such as a fully implantable pump system or an external pump system. Suitable pump and catheter systems are commercially available, e.g., SynchroMed® pump and InDura® intrathecal catheters (both from Medtronic Sofamor Danek, Memphis, Tenn.). The induction and/or maintenance regimen may also be administered as part of an implantable device that comprises a depot formulation of one or more TATs. In some embodiments, the device comprising a depot formulation may take the form of a biodegradable or resorbable substance, including polymers such as poly lactic acid, (PLA), polyglycolic acid (PGA), a hydrogel, and co-polymers of polylactic acid/polyglycolic acid (PLGA). The device comprising a depot formulation may comprise capsules or microcapsules. In a further embodiment, the maintenance regimen may be administered by transfusion, such as IV transfusion.

Compositions, Formulations, and Kits

Compositions and Formulations

Also provided herein are compositions and formulations for use in the described methods. Novel compositions or formulations can be based on the need for particular concentration ranges of a TAT or particular formulation characteristics (e.g., lipophilicity, pH, stability) in the administration regimen chosen. For example, provided herein is a pharmaceutical composition comprising a direct TNF-I at a concentration in the range of from about 1 to about 100 mg/cc, e.g., about 5 to about 50 mg/cc. Such a composition can be useful for the more invasive modes of administration contemplated herein, e.g., intradiskal, peridiskal, epidural, and intrathecal administration. The direct TNF-I for use in the formulation can be any of those previously described, and in some cases is selected from adalimumab, infliximab, CDP-870, CDP-570, etanercept, and pegsunercept. Any of the compositions can further include other agents, including the SAIs described previously.

TAT compositions useful in the practice of the invention comprise at least one TAT, and in the case of small molecule inhibitors, its pharmaceutically acceptable salt, solvate, hydrate, stereoisomer, clathrate, or prodrug thereof. The composition, shape, and type of dosage form will typically vary depending on their use. For example, a parenteral dosage form may contain smaller amounts of one or more of the active ingredients than an oral dosage form used for the same purpose. These and other ways in which specific dosage forms encompassed by this invention will vary from one another will be readily apparent to those skilled in the art. See, e.g., [28]. Typical pharmaceutical compositions and dosage forms also comprise one or more excipients. Suitable excipients are well known to those skilled in the art.

The invention further encompasses the use of compounds that reduce the rate by which the TAT or SAI will decompose. Such compounds, which are referred to herein as “stabilizers,” include, but are not limited to, antioxidants such as ascorbic acid, pH buffers, or salt buffers. The amounts and specific types of stabilizers or other ingredients in a dosage form may differ depending on factors such as, but not limited to, the route by which it is to be administered to patients.

Kits

The present disclosure also contemplates kits for use in the methods described herein. In some embodiments, a kit is provided that includes a syringe, catheter, pump, or delivery device, where the syringe, catheter, pump or delivery device are adapted for epidural or intradiskal administration, and a TAT. The TAT can be disposed within the syringe, catheter, pump, or delivery device and/or can be contained in a vial. The kits can further include other optional ingredients, including an SAI and/or anesthetic (e.g., either or both of which could in a separate vial from the TAT, in the same vial as the TAT, or disposed within the syringe, catheter, pump, or delivery device). A kit can further include a TAT (e.g., a direct TNF-I) disposed within a hydrogel or depot form of administration. In some embodiments, a kit can include a TAT (e.g., a direct TNF-I) at a concentration in the range of from about 1 to about 100 mg/cc, e.g., in the range of from about 5 to about 50 mg/cc.

In other embodiments, the kit may comprise devices or apparatuses that are used to administer the active ingredients. Examples of such devices include, but are not limited to, syringes, needles, catheters, drip bags, patches and inhalers. In some embodiments, the kit might include, for example, some or all of the necessary syringes, needles, catheters and other disposable equipment useful for intrathecal, intradiskal, peridiskal, or epidural placement and administration, either with or without fluoroscopic guidance. Likewise, the kit might contain the necessary syringes, needles, and tubes for IV administration, or for SC administration of the TAT.

In some embodiments, one or more of the active ingredients in the kit might need to be separated from the other components of the kit and refrigerated until the time that the kit is used.

Kits can include without limitation a source of a first TAT and a source of a second TAT (which may be the same or different) and devices/apparatuses to facilitate delivery by different routes, such as intradiskal/epidural injection or IV infusion. Kits of the invention may further comprise pharmaceutically acceptable vehicles that can be used to administer one or more of the active ingredients. For example, if an active ingredient is provided in a solid form that must be reconstituted for parenteral administration, the kit may comprise a sealed container of a suitable vehicle in which the active ingredient can be dissolved to form a particulate-free sterile solution suitable for parenteral administration. Examples of pharmaceutically acceptable vehicles include, but are not limited to, water for injection USP; aqueous vehicles such as, but not limited to, sodium chloride injection, Ringer's Injection, Dextrose Injection, Dextrose and Sodium Chloride Injection, and Lactated Ringer's Injection; water-miscible vehicles such as, but not limited to, ethyl alcohol, polyethylene glycol and polypropylene glycol; and non-aqueous vehicles such as, but not limited to, corn oil, cottonseed oil, peanut oil, ethyl oleate, isopropyl myristate and benzyl benzoate.

Kits can include, optionally, one or more devices, e.g., devices for implantation. Examples of devices include any of the devices described previously, including nucleus replacement devices, annual repair devices; dynamic stabilization devices (pedicle screw or interspinous spacer based); artificial disks; interbody spinal fusion devices; and facet replacement devices (pedicle screw and spacer based). Any of the adjunctive devices or compositions described previously can also be independently included in a kit, e.g., adhesion barriers. In addition, kits can optionally include one or more sources of bone growth stimulatory proteins e.g., BMP-2 disposed within a collagen sponge, a controlled release formulation, or a depot.

### EXAMPLES

### Example 1

### TNF-I Treatment in Subject Eligible for Nucleus Replacement Procedure for Herniated Disk

A subject who is suffering from low back pain and leg pain is seen by his general practitioner (GP), who recommends rest, analgesics, and physical therapy. After 6 weeks, the subject returns to the GP, complaining that the pain has not resolved. The subject is referred by the GP to a spine interventionalist to determine if the subject should undergo a partial or full diskectomy. After evaluating the patient, the spine interventionalist determines that the patient has a herniated disk at L4-L5, that the patient's disk needs supplementation, and that the patient is eligible for nucleus replacement based on the subject meeting the eligibility criteria for a clinical trial of such a procedure, including MRI findings of HD at the appropriate level, the persistent pain of the subject for more than 6 weeks, and the failure of conventional conservative treatment. The spine interventionalist, based on the subject's eligibility for the nucleus replacement, recommends that the subject undergo a course of treatment with a TAT, specifically a TNF-I, to delay the need for the surgery or to improve the outcome of the surgery, should it ultimately result. The spine interventionalist administers the TNF-I intradiskally to the subject and at the same implants a depot formulation of a TNF-I adjacent to the affected spinal NR. The subject is assessed post-administration using one or more of the following: the Roland disability questionnaire, the Oswestry disability questionnaire, the VAS pain scale, the Likert scale, an MRI evaluation, and a neurological assessment.

### Example 2

### TNF-I Treatment in Subject Eligible for Annular Repair Procedure for Herniated Disk

A subject who is suffering from low back pain and leg pain is seen by his GP, who recommends conservative treatment (e.g., rest, analgesics, physical therapy) for a period of 6 weeks. After 6 weeks, the subject returns to the GP, complaining that the pain has not resolved. The subject is referred by the GP to a spine interventionalist to determine if the subject should undergo a partial or full diskectomy. After evaluating the patient, the spine interventionalist determines that the patient has a herniated disk at L5-S1 and is eligible for a partial diskectomy and annular repair device based on the subject meeting the eligibility criteria in a CPG for such a procedure, including MRI findings of HD at the appropriate level, the persistent pain of the subject for more than 6 weeks, and the failure of conventional conservative treatment. The spine interventionalist, based on the subject's eligibility for the procedure recommends that the subject undergo a course of treatment with a TAT, specifically a direct TNF-I such as etanercept, to delay the need for the surgery or to improve the outcome of the surgery, should it ultimately result. The spine interventionalist administers the direct TNF-I intradiskally to the subject as an induction dose, and follows with a maintenance regimen of IV administration of a TNF-I every week for a period of 6 months. The subject is assessed post-administration using one or more of the following: the Roland disability questionnaire, the Oswestry disability questionnaire, the VAS pain scale, the Likert scale, an MRI evaluation, and a neurological assessment.

### Example 3

### TNF-I Treatment in Subject with DDD, Eligible for Dynamic Stabilization with a Pedicle Screw Based Motion Preserving Device

A subject who is suffering from moderate to severe low back pain and leg pain is seen by his GP, who recommends a course of rest, analgesics and physical therapy. After 6 weeks, the subject returns to the GP, complaining that the pain has not resolved. The subject is referred by the GP to a spine interventionalist to determine if the subject should undergo a dynamic stabilization surgical procedure. After evaluating the patient, the spine interventionalist determines that the patient has a herniated disk at L2-L3 and is eligible for a dynamic stabilization procedure with a pedicle screw based device, based on the subject meeting the eligibility criteria of a clinical trial of a new pedicle screw device, including MRI findings of DDD at the appropriate level, the persistent pain of the subject for more than 6 months, and the failure of conventional conservative treatment. The spine interventionalist, based on the subject's eligibility, recommends that the subject undergo a course of treatment with a TAT, specifically a direct TNF-I such as adalimumab, to delay the need for the surgery or to improve the outcome of the surgery, should it ultimately result. The spine interventionalist administers the direct TNF-I intrathecally over a period of one month to the subject using an implanted pump/catheter system. The subject is assessed post-administration using one or more of the following: the Roland disability questionnaire, the Oswestry disability questionnaire, the VAS pain scale, the Likert scale, an MRI evaluation, and a neurological assessment.

### Example 4

### TNF-I Treatment in Subject with DDD, Eligible for Disk Arthroplasty Procedure

A subject suffering from severe back and leg pain, numbness and tingling, and weakness while walking is seen by his GP, who recommends rest, analgesics, and an orthotic brace. After 6 months, the subject returns to the GP, complaining that the symptoms have not resolved. The subject is referred by the GP to a spine interventionalist. After evaluating the patient, based upon MRI findings, the persistent pain of the subject for 6 months, and the failure of conventional conservative treatment, the spine interventionalist diagnoses the subject as suffering from moderate to severe DDD with internal disk derangement, thereby eligible for an artificial disk, based on the subject meeting the eligibility criteria for a clinical trial of disk arthroplasty. The spine interventionalist, based on the subject's eligibility, recommends that the subject undergo an induction/maintenance course of treatment with a TAT, such as a direct TNF-I, to delay the need for the surgery. For the induction phase, the spine interventionalist administers the direct TNF-I epidurally to the subject, local to the site of the affected disk. The subject is then administered a maintenance regimen of a direct TNF-I, where the maintenance regimen includes SC injections every week for a period of 12 weeks, with the dose of each maintenance regimen injection being higher than the initial epidural induction dose. The subject is assessed post-administration using one or more of the following: the Roland disability questionnaire, the Oswestry disability questionnaire, the VAS pain scale, the Likert scale, an MRI evaluation, and a neurological assessment.

### Example 5

### TNF-I Treatment in Subject with DDD, SS, or Grade I or Less Spondylolisthesis Eligible for Interbody Spinal Fusion Procedure

A subject who is suffering from severe back pain, leg weakness, and increased pain upon standing, is seen by his GP, who recommends conservative treatment (e.g., rest, analgesics) for a period of 6 months. The practitioner notes that the patient had 2 years previously had both a diskectomy and laminectomy to alleviate pain. After 6 months of conservative treatment, the subject returns to the GP, complaining that the symptoms have not resolved. The subject is referred by the GP to a spine interventionalist to determine if the subject should undergo an interbody spinal fusion procedure. After evaluating the patient, the spine interventionalist determines that the patient is eligible for an interbody spinal fusion procedure based on the subject meeting the eligibility criteria of a CPG for such a procedure, including MRI findings of DDD and SS at the appropriate level, the persistent pain of the subject for more than 6 months, the failure of conventional conservative treatment, and the failure of the prior decompression procedures. The spine interventionalist, based on the subject's eligibility, recommends that the subject undergo a course of treatment with a TAT, specifically a direct TNF-I such as etanercept, to improve the outcome of the surgery. At a time period of 2 weeks before the procedure, the spine interventionalist administers the direct TNF-I intradiskally and peridiskally to the subject, in the regions of the SS and DDD. After 2 weeks, the spine interventionalist then performs the surgery, and starting at 2 weeks post surgery, the subject is administered a TAT SC every 1 week for a period of 24 weeks. The post-surgery SC doses are all at a higher dose per administration than the pre-surgery dose. The subject is assessed post-administration using one or more of the following: the Roland disability questionnaire, the Oswestry disability questionnaire, the VAS pain scale, the Likert scale, an MRI evaluation, and a neurological assessment.

### Example 6

### NFκB-I Treatment to Prevent Adjacent Level Disease in Subject Who has Undergone a Single or Multi-Level Interbody Spinalfusion Procedure

A subject who is suffering from severe back pain, leg weakness, and increased pain upon walking, is seen by his GP, who recommends conservative treatment (e.g., rest, analgesics) for a period of 6 months. The practitioner notes that the subject had a diskectomy the year before to alleviate pain. After 6 months of conservative treatment, the subject returns to the GP, complaining that the symptoms have not resolved. The subject is referred by the GP to a spine interventionalist to determine if the subject should undergo an interbody spinal fusion procedure. After evaluating the patient, the spine interventionalist determines that the patient is eligible for an interbody spinal fusion procedure based on the subject meeting the eligibility criteria of a CPG for such a procedure, including MRI findings of DDD and SS at the appropriate level, the persistent pain of the subject for more than 6 months, the failure of conventional conservative treatment, and the failure of the prior decompression procedure. The spine interventionalist, based on the subject's eligibility, recommends that the subject undergo a course of treatment with a TAT, specifically an NFκB-I, to improve the outcome of the surgery by reducing pain and inflammation, retarding further disk degeneration, and thereby preventing or reducing the development of adjacent level disease. At a time period of 2 weeks before the procedure, the spine interventionalist administers the direct NFκB-I epidurally to the subject, in the regions of the SS and degenerated disk. After 2 weeks, the spine interventionalist then performs the surgery, and starting at 2 weeks post surgery, the subject is administered a TAT SC every 2 weeks for a period of 2 years. The post-surgery SC doses are all at a higher dose per administration than the pre-surgery dose. The subject is assessed post-administration using one or more of the following: the Roland disability questionnaire, the Oswestry disability questionnaire, the VAS pain scale, the Likert scale, an MRI evaluation, and a neurological assessment.

### Example 7

### TNF-I Treatment of BMP-Induced Radiculitis in Subject Who Has Undergone an Interbody Spinalfusion Procedure

A subject who is suffering from severe back pain, leg weakness, and increased pain upon walking, is seen by his GP, who recommends conservative treatment (e.g., rest, analgesics) for a period of 6 months. The practitioner notes that the patient had had a diskectomy the year before to alleviate pain. After 6 months of conservative treatment, the subject returns to the GP, complaining that the symptoms have not resolved. The subject is referred by the GP to a spine interventionalist to determine if the subject should undergo an interbody spinal fusion procedure. After evaluating the patient, the spine interventionalist determines that the patient is eligible for an interbody spinal fusion procedure based on the subject meeting the eligibility criteria of a CPG for such a procedure, including MRI findings of DDD and SS at the appropriate level, the persistent pain of the subject for more than 6 months, the failure of conventional conservative treatment, and the failure of the prior decompression procedure. The interventionalist performs the interbody spinal fusion procedure using BMP-2 instead of autogenous iliac crest autograft. The subject experiences severe leg pain within 12 hours of the completion of the surgery. The interventionalist administers a perispinal dose of a direct TNF-I, and follows up with a maintenance regimen of a direct TNF-I administered SC every 2 weeks for a period of six months. The SC doses are all at a higher dose per administration than the induction dose. The subject is assessed post-administration using one or more of the following: the Roland disability questionnaire, the Oswestry disability questionnaire, the VAS pain scale, the Likert scale, an MRI evaluation, and a neurological assessment.

### Example 8

### TNF-I/NFkB-I Combination Treatment in Subject with FBSS

A subject that has had a previous interbody spinal fusion procedure returns to his spine interventionalist 6 months after the fusion procedure, complaining of the same back and leg pain as before the procedure. The spine interventionalist recommends conservative treatment (e.g., rest, analgesics) for a period of 6 months. After 6 months of conservative treatment, the subject returns to the spine interventionalist, complaining that the symptoms have not resolved. After evaluating the patient, the spine interventionalist determines that the patient has FBSS, as evidenced by the subject's continued level of pain and failure of conservative treatment, and therefore meets the eligibility criteria of a CPG for repeat or revision fusion procedure. The spine interventionalist, based on the subject's eligibility for a repeat or revision fusion procedure, recommends that the subject undergo an induction/maintenance course of treatment with two TATs, a direct TNF-I and an NFκB-I, to delay the need for the surgery. For the induction phase, the spine interventionalist administers the TNF-I intradiskally/peridiskally to the site of pathology. For the maintenance regimen, the interventionalist administers NFκB-I epidurally to the subject, local to the site of the affected pathology, every week for a period of 12 weeks, with the dose of each maintenance regimen injection being higher than the initial intradiskal/peridiskal induction dose. The subject is assessed post-administration using one or more of the following: the Roland disability questionnaire, the Oswestry disability questionnaire, the VAS pain scale, the Likert scale, an MRI evaluation, and a neurological assessment.

### Example 9

### TNF-I Administration in Subject Eligible for NR Revision or Replacement

A subject that had a nucleus replacement device implanted 8 months earlier returns to his spine interventionalist complaining of the same level and type of pain as before the procedure. The spine interventionalist recommends conservative care for a period of 6 months. After 6 months, the subject returns to the spine interventionalist complaining that the pain has not resolved. After examining the subject and reviewing the history, the interventionalist determines that the subject is eligible for a revision or replacement procedure as evidenced by the subject's continued level of pain, failure of conservative treatment, and confirmation of reduced disk height and DDD through radiologic assessment. The spine interventionalist, based on the subject's eligibility for a repeat or revision nucleus replacement procedure, recommends administering a direct TNF-I via an intradiskal/peridiskal/epidural administration. The revision/replacement procedure proceeds 2 weeks later. During the procedure, the interventionalist sprays a direct TNF-I into the surgical spine wound (intra-operative administration) as an induction dose. After the procedure, the interventionalist follows up with a maintenance regimen of perispinal injections of a direct TNF-I every 2 weeks for a period of 3 months.

### Example 10

### TNF-I Treatment to Improve the Outcome of Decompression Surgery in a Subject with HD Undergoing Diskectomy, with an Anti-Adhesive Gel

A subject who is suffering from leg pain is seen by his GP, who recommends conservative treatment (e.g., rest, analgesics, physical therapy) for a period of 12 weeks. After 12 weeks, the subject returns to the GP, complaining that the pain has not resolved. The subject is referred by the GP to a spine interventionalist to determine if the subject should undergo a partial or full diskectomy. After evaluating the patient, the spine interventionalist determines that the patient has a herniated disk at L3-L4 and is eligible for a full diskectomy based on the subject meeting the NASS guideline [4] for such a procedure, including MRI findings of HD at the appropriate level, the persistent pain of the subject for more than 12 weeks, and the failure of conventional conservative treatment. The spine interventionalist decides to perform the surgery, and administers a direct TNF-I epidurally 2 weeks prior to surgery. In conjunction with the diskectomy procedure, the spine interventionalist applies an anti-adhesion gel directly in the dural space and epidiskally, to prevent fibrotic adhesions from forming post-surgery. In addition, the spine interventionalist recommends that the subject undergo a course of treatment with an HSCI, specifically a direct TNF-I such as etanercept, to improve the outcome of the diskectomy surgery. The spine interventionalist administers the direct TNF-I intradiskally and peridiskally to the subject 2 weeks prior to the diskectomy procedure. Starting at 2 weeks post surgery, the subject is administered an HSCI subcutaneously every 2 weeks for a period of 24 weeks. The subject is assessed post-administration using one or more of the following: the Roland disability questionnaire, the Oswestry disability questionnaire, the VAS pain scale, the Likert scale, an MRI evaluation, and a neurological assessment.

A number of embodiments of the invention have been described. Nevertheless, it will be understood that various modifications may be made without departing from the spirit and scope of the invention. Accordingly, other embodiments are within the scope of the following claims.

### REFERENCES

1 Shin K C et al. (2005). A prospective controlled trial of TNF-alpha inhibitor for symptomatic patients with cervical disk herniation. Spine J. 5: 45S.
2 Korhonen T et al. (2005). The treatment of disc-herniation-induced sciatica with infliximab: results of a randomized, controlled, 3-month follow-up study. Spine 30:2724-28.
3 North American Spine Society (2000). Phase III Clinical Guidelines for Multidisciplinary Spine Care Specialists. Spondylolysis, Lytic Spondylolisthesis and Degenerative Spondyolisthesis (SLD) Version 1.0. NASS: La Grange, Ill.
4 North American Spine Society (2000). Phase III Clinical Guidelines for Multidisciplinary Spine Care Specialists. Herniated Disc Version 1.0. NASS: La Grange, Ill.
5 Di Martino A et al. (2005). Nucleus pulposus replacement: basic science and indications for clinical use. Spine 30(16 Suppl):S16-22.
6 Annulex Technologies Corporate Brochure (2007).
7 Applied Spine Technologies (2007). Dynamic Stabilization for Lumbar Spinal Stenosis With Stabilimax NZ® Dynamic Spine Stabilization System. Clinical Trials.gov Identifier: NCT00479544. (http://www.clinicaltrials.gov/ct/show/NCT00479544?order=1) Updated May 2007
8 Medtronic Spine (2007). Study of the Safety and Effectiveness of DIAM™ Spinal Stabilization System vs. Conservative Care. Clinical Trials.gov Identifier: NCT00456378. (http://www.clinicaltrials.gov/ct/show/NCT00479544?order=42) Updated July 2007
9 Abbot Spine (2007). Wallis Stabilization System for Low Back Pain. ClinicalTrials.gov Identifier: NCT00484458. (http://www.clinicaltrials.gov/ct/show/NCT00483358?order=1) Updated June 2007
10 Zucherman J F et al. (2004). A multicenter, prospective, randomized trial evaluating the X STOP interspinous process decompression system for the treatment of neurogenic intermittent claudication. Spine 30: 1351-1358.
11 DePuy Spine (2006). Comparison of DISCOVER™ Artificial Cervical Disc and ACDF for Treatment of Cervical DDD (IDE Study). ClinicalTrials.gov Identifier: NCT00432159. http://www.clinicaltrials.gov/ct/show/NCT00432159?order=3
12 Blumenthal S et al. (2005). A prospective, randomized, multicenter Food and Drug Administration investigational device exemptions study of lumbar total disk replacement with the CHARITE artificial disc versus lumbar fusion: part I:

evaluation of clinical outcomes. Spine. 30:1565-1575.
13 Sasso R C et al. (2004). A prospective, randomized controlled clinical trial of anterior lumbar interbody fusion using a titanium cylindrical threaded fusion device. Spine 29:113-122; discussion 121-122.
14 Videbaek T S et al. (2006). Circumferential fusion improves outcome in comparison with instrumented posterolateral fusion: long-term results of a randomized clinical trial. Spine 31:2875-2880.
15 Burkus J K et al. (2002). Clinical and radiographic outcomes of anterior lumbar interbody fusion using recombinant human bone morphogenetic protein-2. Spine 27:2396-2408.
16 Vaccaro A R et al. (2005). Comparison of OP-1 Putty (rhBMP-7) to iliac crest autograft for posterolateral lumbar arthrodesis: a minimum 2-year follow-up pilot study. Spine 30:2709-2716.
17 Garfin S R et al. (2006). Balloon kyphoplasty for symptomatic vertebral body compression fractures results in rapid, significant, and sustained improvements in back pain, function, and quality of life for elderly patients. Spine 31:2213-2220.
18 Kyphon Corp (2006). KAVIAR Study—Kyphoplasty And Vertebroplasty In the Augmentation and Restoration of Vertebral Body Compression Fractures. ClinicalTrials.gov Identifier: NCT00323609. (http://www.clinicaltrials.gov/ct/show/NCT00323609?order=1)
19 Archus Orthopedics, Inc. (2005). Total Facet Arthroplasty System® (TFAS®) Clinical Trial. ClinicalTrials.gov Identifier: NCT00418197. (http://www.clinicaltrials.gov/ct/show/NCT00418197?order=2) Updated March 2007
20 Integra Life Sciences (2006). DuraGen Plus® Adhesion Barrier for Use in Spinal Surmgery. ClinicalTrials.gov Identifier: NCT00387829. (http://www.clinicaltrials.gov/ct/show/NCT00387829?order=7)
21 Kim K D et al. (2003). Reduction of Radiculopathy and Pain with Oxiplex/SP Gel Following Laminectomy, Laminotomy, and Discectomy: A Pilot Clinical Study. Spine 28:1080-1088.
22 Armour K L et al. (1999). Recombinant human IgG molecules lacking Fcgamma receptor I binding and monocyte triggering activities. Eur. J. Immunol. 29:2613-2624.
23 De A U and Pal D (1975). Possible antineoplastic agents I. J. Pharm. Sci. 64(2):262-266.
24 List A et al. (2005). Efficacy of Lenalidomide in Myelodysplastic Syndromes. N. Eng. J. Med. 352:549-57.
25 Muto M (2007). Intradiscal and Intraforaminal Oxygen-ozone Therapy in the Treatment of Herniated Disks. Abstract presented at the American Society of Spine Radiology Annual Symposium, February 22-25 in Marco Island, Fla.
26 PDF Staff (2003). Physicians' Desk Reference, 57^{th} ed. 594-597. Montvale, N.J.: Thomson Healthcare
27 Lamer T J (1994). Mayo Clinic Proceedings. 69:473-480.
28 Gennard, A R, editor (1990). Remington's Pharmaceutical Sciences, 18th edition. Easton, Pa.: Mack Publishing Company.

What is claimed is:

1. A method of identifying a subject who could benefit therapeutically from administration of a direct TNF inhibitor (direct TNF-I), the method comprising determining that the subject meets at least one predetermined standard of eligibility (SOE) for a spinal device or fusion procedure, thereby identifying the subject as one who could benefit.

2. A method of identifying a subject who could benefit therapeutically from administration of an NFκB inhibitor (NFκB-I), the method comprising determining that the subject meets at least one predetermined SOE for a spinal device or fusion procedure, thereby identifying the subject as one who could benefit.

3. The method of claim 1 or 2, wherein the subject is:
a) eligible for a disk nucleus replacement procedure;
b) eligible for an annular repair procedure;
c) eligible for a dynamic stabilization procedure;
d) eligible for an artificial disk procedure;
e) eligible for an interbody spine fusion;
f) eligible for a posterolateral fusion;
g) eligible for an interbody spine fusion using BMP-2;
h) eligible for kyphoplasty, vertebroplasty or vertebral restoration;
i) eligible for facet replacement; or
j) eligible for spinal procedure augmented by an anti-adhesive.

4. The method of claim 1 or 2, wherein the predetermined SOE is selected from:
a) a determination of eligibility of the subject for the spinal device or fusion procedure by a healthcare service provider, as evidenced by:
i) a scheduling or request for scheduling by a healthcare service provider of the spinal device or fusion procedure for the subject;
ii) a communication by a healthcare service provider to the subject that the subject has been determined to be eligible for the spinal device or fusion procedure;
iii) a provision or offering by a healthcare service provider to the subject of a consent form for the spinal device or fusion procedure;
iv) a receipt or execution by the subject of a consent form for the spinal device or fusion procedure, said consent form provided by the subject's healthcare provider; or
v) a notation by the healthcare service provider in a tangible medium that the patient is eligible for the spinal device or fusion procedure;
b) a determination of eligibility of the subject for the spinal device or fusion procedure by a qualified entity other than the subject's healthcare provide; and
c) the meeting by the subject of the eligibility criteria for a spinal device or fusion procedure in one or more CPG(s) or clinical trial(s).

5. The method of claim 1, further comprising recording said identification of said subject in a tangible medium.

6. The method of claim 1, further comprising administering a direct TNF-I to the subject.

7. The method of claim 2, further comprising administering an NFκB-I to the subject.

8. The method of claim 6, wherein the direct TNF-I is selected from the group consisting of an antibody or antibody fragment, a fusion protein, a peptide, a SMIP, a small molecule, an oligonucleotide, an oligosaccharide, a soluble cytokine receptor or fragment thereof, a soluble TNF receptor Type I or a functional fragment thereof, a polypeptide that binds to TNF, and a dominant negative TNF molecule.

9. The method of claim 8, wherein the oligonucleotide is an siRNA.

10. The method of claim 8, wherein the direct TNF-I is selected from the group consisting of: Humira® (adalimumab/D2E7); Remicade® (infliximab); Cimzia® (CDP-870); Humicade® (CDP-570); golimumab (CNTO 148); CytoFab (Protherics); AME-527; anti-TNF-Receptor 1 mAb or dAb; ABX-10131; polyclonal anti-TNF antibodies; anti-TNF polyclonal anti-serum; anti-TNF or anti-TNF-R SMIPs (Trubion); Enbrel® (etanercept); pegsunercept/PEGs TNF-R1, onercept; recombinant TNF binding protein (r-TBP-1); trimerized TNF antagonist; SSR-150106 (Sanofi-Synthelabo); ABX-0402 (Ablynx); nanobody therapeutics (Ablynx); trimerized TNF antagonist (Borean); humanized anti-TNF mAb (Biovation); Dom-0200 (Domantis); Genz-29155 (Genzyme); agarooligosaccharide (Takara Shuzo); HTDN-TNF (Xencor); and therapeutic human polyclonal anti-TNF and anti-TNF-R antibodies (THP).

11. The method of claim 7, wherein the NFκB-I is selected from the group consisting of sulfasalazine, sulindac, clonidine, helenalin, wedelolactone, pyrollidinedithiocarbamate (PDTC), IKK-2 inhibitors, and IKK inhibitors.

12. A method for preventing or postponing a spinal device or fusion procedure in a subject wherein the subject meets at least one predetermined SOE for a spinal device or fusion procedure, the method comprising:
a) optionally identifying the subject as a subject eligible for the spinal device or fusion procedure;
b) administering to the subject a therapeutically effective amount of at least one direct TNF-I; and
c) optionally determining whether the subject's eligibility for the spinal device or fusion procedure has been prevented or postponed.

13. A method for preventing or postponing a spinal device or fusion procedure in a subject wherein the subject meets at least one predetermined SOE for a spinal device or fusion procedure, the method comprising:
a) optionally identifying the subject as a subject eligible for the spinal device or fusion procedure;
b) administering to the subject a therapeutically effective amount of at least one NFκB-I; and
c) optionally determining whether the subject's eligibility for the spinal device or fusion procedure has been prevented or postponed.

14. The method of claim 12 or 13, wherein the subject is:
a) eligible for a disk nucleus replacement procedure;
b) eligible for an annular repair procedure;
c) eligible for a dynamic stabilization procedure;
d) eligible for an artificial disk procedure;
e) eligible for an interbody spine fusion;
f) eligible for a posterolateral fusion;
g) eligible for an interbody spine fusion using BMP-2;
h) eligible for kyphoplasty, vertebroplasty or vertebral restoration;
i) eligible for facet replacement; or
j) eligible for a spinal procedure augmented by an anti-adhesive.

15. The method of claim 12 or 13, wherein the predetermined SOE is selected from:
a) a determination of eligibility of the subject for the spinal device or fusion procedure by a healthcare service provider, as evidenced by:
i) a scheduling or request for scheduling by a healthcare service provider of the spinal device or fusion procedure for the subject;
ii) a communication by a healthcare service provider to the subject that the subject has been determined to be eligible for the spinal device or fusion procedure;
iii) a provision or offering by a healthcare service provider to the subject of a consent form for the spinal device or fusion procedure;
iv) a receipt or execution by the subject of a consent form for the spinal device or fusion procedure, said consent form provided by the subject's healthcare provider; or
v) a notation by the healthcare service provider in a tangible medium that the patient is eligible for the spinal device or fusion procedure;
b) a determination of eligibility of the subject for the spinal device or fusion procedure by a qualified entity other than the subject's healthcare provide; and
c) the meeting by the subject of the eligibility criteria for a spinal device or fusion procedure in one or more CPG(s) or clinical trial(s).

16. The method of claim 12 or 13, wherein the subject is eligible for a disk nucleus replacement procedure based on the subject:
1) having been diagnosed with a) HD confirmed on MRI or b) mild to moderate DDD confirmed on MRI with a loss of disk height of less than 50 percent; and
2) having failed conservative treatment for a period of at least 6 weeks; having back or leg pain from L2-S1 with nerve root involvement or radicular neck pain; and not having facet arthropathy, SS, or spinal segment instability.

17. The method of claim 12 or 13, wherein the subject is eligible for an annular repair procedure based on:
1) the subject having been diagnosed as having HD with MRI and/or CT confirmation and associated leg pain; and the subject having failed conservative treatment for a period of at least 6 weeks; or
2) the subject is undergoing nucleus replacement, and the treating spine interventionalist elects to perform conjoint annular repair.

18. The method of claim 12 or 13, wherein the subject is eligible for a dynamic stabilization procedure with a pedicle screw based device based on:
1) the subject having been diagnosed with one or more of the following: a) mild to moderate DDD; b) moderate to severe SS with back or leg pain from L2-S1; where either the DDD or stenosis is confirmed by MRI and/or CT; and c) pain originating from the disk, facet joints, and/or ligaments confirmed by physical/neurological examination; and
2) the failure of conservative treatment for a period of at least 6 months.

19. The method of claim 12 or 13, wherein the subject is eligible for a dynamic stabilization spinal procedure with an interspinous process spacer based on:
A) the subject having been diagnosed with one of the following: 1) a) mild to moderate DDD or b) moderate to severe SS with back or leg pain from L2-S1, where either the DDD or stenosis is be confirmed by MRI and/or CT; and
B) the subject is experiencing a) intermittent neurogenic claudication or b) low back pain improving with flexion, or c) radicular leg pain; and
C) the failure of conservative treatment for a period of at least 6 months.

20. The method of claim 12 or 13, wherein the subject is eligible for an artificial disk procedure based on:
A) the subject having been diagnosed with moderate to severe DDD confirmed by MRI and/or CT and wherein the subject does not have severe facet arthropathy, gross spine instability, or vertebral body osteoporosis;
wherein, for lumbar artificial disk procedures, the subject also experiences back or leg pain with provocative diskography and has failed at least 6 months of conservative therapy; and
wherein, for cervical artificial disk procedures, the subject also experiences radiculopathy manifesting as neck or arm pain or a decrease in muscle strength and has failed conservative therapy for a minimum of 6 weeks.

21. The method of claim 12 or 13, wherein the subject is eligible for an interbody spine fusion procedure based on:
A) the subject having been diagnosed with DDD and one or more of the following: a) moderate to severe spinal instability; b) SS; and c) spondylolisthesis, with the diagnosis confirmed by either CT, and/or MRI, and/or x-ray; and
B) the subject has back or neck pain that has failed conservative treatment for a minimum of 6 months.

22. The method of claim 12 or 13, wherein the subject is eligible for a posterolateral fusion based on:
A) the subject having been diagnosed with a) DDD with degenerative spondylolisthesis and/or b) SS, with the diagnosis confirmed by MRI and/or CT; and
B) the subject has low back pain that has failed conservative treatment for a period of at least 6 months.

23. The method of claim 12 or 13, wherein the subject is eligible for an interbody spine fusion procedure using BMP-2 based on:
A) the subject having been diagnosed with DDD and one or more of the following: a) moderate to severe spinal instability; b) SS; and c) spondylolisthesis, with the diagnosis confirmed by either CT, and/or MRI, and/or x-ray; and
B) the subject has back or neck pain that has failed conservative treatment for a minimum of 6 months.

24. The method of claim 12 or 13, wherein the subject is eligible for a kyphoplasty, vertebroplasty or vertebral restoration based on
A) the subject having been diagnosed with a vertebral compression fracture confirmed on x-ray, CT and/or MRI; and
B) the subject experiences back pain correlated with the site of the vertebral compression fracture.

25. The method of claim 12 or 13, wherein the subject is eligible for a facet replacement procedure based on:
A) the subject having been diagnosed with facet arthritis confirmed by CT and/or MRI and optionally with degenerative SS; and
B) the subject experiences intermittent neurogenic claudication that worsens on walking or standing, coupled with radiological evidence of nerve root impingement by either osseous or non-osseous elements.

26. The method of claim 12 or 13, wherein the subject is eligible for a spinal procedure involving implantation of an anti-adhesive based on:
A) the subject being eligible for a spinal device or fusion procedure selected from the following:
a) a disk nucleus replacement procedure;
b) an annular repair device procedure;
c) a dynamic stabilization procedure;
d) an artificial disk procedure;
e) an interbody spine fusion;
f) a posterolateral fusion;
g) an interbody spine fusion using BMP-2;
h) a kyphoplasty, vertebroplasty or vertebral restoration; and
i) facet replacement; or
B) the subject being eligible for any spinal device or fusion procedure that does not involve the implantation of an implantable device or fusion of vertebrae.

27. The method of claim 26, wherein the spinal device or fusion procedure that does not involve the implantation or fusion is selected from diskectomy, laminectomy, percutaneous or endoscopic epidural adhesiolysis, radiofrequency neurotomy (RFN), and intradiskal electrothermal therapy (IDET)

28. The method of claim 12 or 13, further comprising objectively or subjectively assessing the effect of step b) on the subject, wherein the assessment comprises at least one of the following steps:
a) determining a level or temporal duration of pain, impaired mobility, disability, or spinal nerve root irritation in the subject;
b) determining an amount of TNF in the subject at a location of interest;
c) fluoroscopically or radiologically observing the subject;
d) determining whether the subject continues to meet the eligibility criteria in the predetermined SOE or CPG for the spinal device or fusion procedure;
e) determining a measure of disability using the Oswetry Disability Index;
f) determining a measure of functioning using the Short Form 36 Assay;
e) optionally comparing the results of any one of steps a) to f) with the results of the same step performed prior to the step described in claim 12b) or 13b).

29. The method of claim 12, wherein step b) comprises at least 2 separate administrations of a direct TNF-I.

30. The method of claim 13, wherein step b) comprises at least 2 separate administrations of an NFκB-I.

31. The method of claim 12, wherein the administration in b) treats the subject so that the subject does not undergo a spinal device or fusion procedure in at least the first three months after the initial administration of the TNF-I.

32. The method of claim 13, wherein the administration in b) treats the subject so that the subject does not undergo a spinal device or fusion procedure in at least the first three months after the initial administration of the NFκB-I.

33. The method of claim 12, wherein the direct TNF-I is selected from the group consisting of an antibody or antibody fragment, a fusion protein, a peptide, a SMIP, a small molecule, an oligonucleotide, an oligosaccharide, a soluble cytokine receptor or fragment thereof, a soluble TNF receptor Type I or a functional fragment thereof, a polypeptide that binds to TNF, and a dominant negative TNF molecule.

34. The method of claim 33, wherein the oligonucleotide is an siRNA.

35. The method of claim 33, wherein the direct TNF-I is selected from the group consisting of: Humira® (adalimumab/D2E7); Remicade® (infliximab); Cimzia® (CDP-870); Humicade® (CDP-570); golimumab (CNTO 148); CytoFab (Protherics); AME-527; anti-TNF-Receptor 1 mAb or dAb; ABX-10131; polyclonal anti-TNF antibodies; anti-TNF polyclonal anti-serum; anti-TNF or anti-TNF-R SMIPs (Trubion); Enbrel® (etanercept); pegsunercept/PEGs TNF-R1, onercept; recombinant TNF binding protein (r-TBP-1); trimerized TNF antagonist; SSR-150106 (Sanofi-Synthelabo); ABX-0402 (Ablynx); nanobody therapeutics (Ablynx); trimerized TNF antagonist (Borean); humanized anti-TNF mAb (Biovation); Dom-0200 (Domantis); Genz-29155 (Genzyme); agarooligosaccharide (Takara Shuzo); HTDN-TNF (Xencor); and therapeutic human polyclonal anti-TNF and anti-TNF-R antibodies (THP).

36. The method of claim 13, wherein the NFκB-I is selected from the group consisting of sulfasalazine, sulindac, clonidine, helenalin, wedelolactone, pyrollidinedithiocarbamate (PDTC), IKK-2 inhibitors, and IKK inhibitors.

37. The method of claim 12, wherein the administration comprises: (a) an induction regimen comprising a direct TNF-I; and (b) a maintenance regimen comprising a direct TNF-I.

38. The method of claim 13, wherein the administration comprises: (a) an induction regimen comprising an NFκB-I; and (b) a maintenance regimen comprising an NFκB-I.

39. The method of claim 37 or 38, wherein the induction regimen is administered intrathecally, intradiskally, peridiskally, or epidurally, or combinations thereof.

40. The method of claim 37 or 38, wherein the maintenance regimen comprises systemic or parenteral administration.

41. The method of claim 37 or 38, wherein the maintenance regimen comprises IV, perispinal, intramuscular, SC, or transdermal administration.

42. The method of claim 37 or 38, wherein the maintenance regimen is administered by a pump.

43. The method of claim 37 or 38, wherein the maintenance regimen is administered by implantation of a depot formulation or a hydrogel formulation.

44. The method of claim 37 or 38, wherein the induction regimen is completed prior to beginning administration of the maintenance regimen.

45. The method of claim 37 or 38, wherein the maintenance regimen begins at or near the same time as the induction regimen.

46. The method of claim 37 or 38, wherein the induction regimen route of administration is selected from intra-operative, intrathecal, intradiskal, peridiskal, epidural (including periradicular and transforaminal), and the maintenance regimen route of administration is selected from perispinal, IV, SC, intramuscular, and transdermal.

47. The method of claim 37 or 38, wherein the induction regimen is administered locally to a site of the spine pathology of the subject, and wherein the maintenance regimen is administered systemically or parenterally.

48. The method of claim 47, wherein the induction regimen comprises a lower dose per administration to the subject than the maintenance regimen dose per administration.

49. The method of claim 47, wherein the induction regimen is administered intrathecally, intradiskally, peridiskally, or epidurally, or any combination thereof.

50. The method of claim 47, wherein the induction regimen is administered within 10 cm of the site of the spinal pathology.

51. The method of claim 6, 7, 12, or 13, further comprising administering to the subject a therapeutically effective amount of a supplemental active ingredient (SAI).

52. The method of claim 51, wherein the SAI is selected from the group consisting of a second TAT, a corticosteroid, ozone, an antirheumatic drug, an LA, a neuroprotective agent, a salicylic acid acetate, a hydromorphone, a non-steroidal anti-inflammatory drug, a cox-2 inhibitor, an antidepressant, an anticonvulsant, a calcium channel blocker, and an antibiotic.

53. The method of claim 12, wherein the direct TNF-I is administered locally to a site of spine pathology of the subject.

54. The method of claim 13, wherein the NFκB-I is administered locally to a site of spine pathology of the subject.

55. The method of claim 12 or 13, wherein the route of administration is selected from the group consisting of intra-operative, intrathecal, intradiskal, peridiskal, epidural (including periradicular and transforaminal), any combination of intradiskal, epidural, and peridural, perispinal, IV, intramuscular, SC, oral, intranasal, inhalation, and transdermal.

56. The method of claim 53, wherein the route of administration is selected from intradiskally, peridiskally, or epidurally, or any combination thereof.

57. The method of claim 54, wherein the route of administration is selected from intradiskally, peridiskally, or epidurally, or any combination thereof.

58. The method of claim 53 or 54, wherein the inhibitor is administered within 10 cm of the site of the spine pathology.

59. A method for improving the outcome of a spinal device or fusion procedure in a subject, wherein the subject meets at least one predetermined SOE for a spinal device or fusion procedure, the method comprising:
a) optionally identifying the subject as a subject eligible for the spinal device or fusion procedure;
b) administering to the subject a therapeutically effective amount of at least one direct TNF-I; and
c) performing the spinal device or fusion procedure, wherein the spinal device or fusion procedure is selected from a spinal device or fusion procedure that implants one or more of an annular repair or replacement device, a dynamic stabilization device, a kyphoplasty/vertebroplasty/vertebral restoration device, a facet replacement and fixation device, a dural repair device, or a spine fusion device.

60. A method for improving the outcome of a spinal device or fusion procedure in a subject, wherein the subject meets at least one predetermined SOE for a spinal device or fusion procedure, the method comprising:
a) optionally identifying the subject as a subject eligible for the spinal device or fusion procedure;
b) administering to the subject a therapeutically effective amount of at least one NFκB-I; and
c) performing the spinal device or fusion procedure, wherein the spinal device or fusion procedure is selected from a spinal device or fusion procedure that implants one or more of an annular repair or replacement device, a dynamic stabilization device, a kyphoplasty/vertebroplasty/vertebral restoration device, a facet replacement and fixation device, a dural repair device, or a spine fusion device.

61. The method of claim 59 or 60, wherein the subject is:
a) eligible for an annular repair procedure;
b) eligible for a dynamic stabilization procedure;
c) eligible for an interbody spine fusion;
d) eligible for an interbody spine fusion using BMP-2;
d) eligible for a posterolateral fusion;
e) eligible for kyphoplasty, vertebroplasty or vertebral restoration; or
f) eligible for facet replacement.

62. The method of claim 59 or 60, wherein the predetermined SOE is selected from:
a) a determination of eligibility of the subject for the spinal device or fusion procedure by a healthcare service provider, as evidenced by:
i) a scheduling or request for scheduling by a healthcare service provider of the spinal device or fusion procedure for the subject;
ii) a communication by a healthcare service provider to the subject that the subject has been determined to be eligible for the spinal device or fusion procedure;
iii) a provision or offering by a healthcare service provider to the subject of a consent form for the spinal device or fusion procedure;
iv) a receipt or execution by the subject of a consent form for the spinal device or fusion procedure, said consent form provided by the subject's healthcare provider; or
v) a notation by the healthcare service provider in a tangible medium that the patient is eligible for the spinal device or fusion procedure;
b) a determination of eligibility of the subject for the spinal device or fusion procedure by a qualified entity other than the subject's healthcare provide; and
c) the meeting by the subject of the eligibility criteria for a spinal device or fusion procedure in one or more CPG(s) or clinical trial(s).

63. The method of claim 59 or 60, wherein the subject is eligible for an annular repair procedure based on:
1) the subject having been diagnosed as having HD with MRI and/or CT confirmation and associated leg pain; and the subject having failed conservative treatment for a period of at least 6 weeks; or
2) the subject is undergoing nucleus replacement, and the treating spine interventionalist elects to perform conjoint annular repair.

64. The method of claim 59 or 60, wherein the subject is eligible for a dynamic stabilization procedure with a pedicle screw based based on:
1) the subject having been diagnosed with one or more of the following: a) mild to moderate DDD; b) moderate to severe SS with back or leg pain from L2-S1; where either the DDD or stenosis is confirmed by MRI and/or CT; and c) pain originating from the disk, facet joints, and/or ligaments confirmed by physical/neurological examination; and
2) the failure of conservative treatment for a period of at least 6 months.

65. The method of claim 59 or 60, wherein the subject is eligible for a dynamic stabilization spinal procedure with an interspinous process spacer based on:
A) the subject having been diagnosed with one of the following: 1) a) mild to moderate DDD or b) moderate to severe SS with back or leg pain from L2-S1, where either the DDD or stenosis is be confirmed by MRI and/or CT; and
B) the subject is experiencing a) intermittent neurogenic claudication, or b) low back pain with improvement on flexion, or c) radicular leg pain; and
C) the failure of conservative treatment for a period of at least 6 months.

66. The method of claim 59 or 60, wherein the subject is eligible for an interbody spine fusion procedure based on:
A) the subject having been diagnosed with DDD and one or more of the following: a) moderate to severe spinal instability; b) SS; and c) spondylolisthesis, with the diagnosis confirmed by either CT and/or MRI, or x-ray; and
B) the subject has back or neck pain that has failed conservative treatment for a minimum of 6 months.

67. The method of claim 59 or 60, wherein the subject is eligible for a posterolateral fusion based on:
A) the subject having been diagnosed with a) DDD with degenerative spondylolisthesis and/or b) SS, with the diagnosis confirmed by MRI and/or CT; and
B) the subject has low back pain that has failed conservative treatment for a period of at least 6 months.

68. The method of claim 59 or 60, wherein the subject is eligible for an interbody spine fusion procedure using BMP-2 based on:
A) the subject having been diagnosed with DDD and one or more of the following: a) moderate to severe spinal instability; b) SS; and c) spondylolisthesis, with the diagnosis confirmed by either CT and/or MRI, and/or x-ray; and
B) the subject has back or neck pain that has failed conservative treatment for a minimum of 6 months.

69. The method of claim 59 or 60, wherein the subject is eligible for a kyphoplasty, vertebroplasty or vertebral restoration based on
A) the subject having been diagnosed with a vertebral compression fracture confirmed on x-ray, CT and/or MRI; and
B) the subject experiences back pain correlated with the site of the vertebral compression fracture.

70. The method of claim 59 or 60, wherein the subject is eligible for a facet replacement procedure based on:
A) the subject having been diagnosed with facet arthritis confirmed by CT and/or MRI and optionally with degenerative SS; and
B) the subject experiences intermittent neurogenic claudication that worsens on walking or standing, coupled with radiological evidence of nerve root impingement by either osseous or non-osseous elements.

71. The method of claim 59, wherein the direct TNF-I is selected from the group consisting of an antibody or antibody fragment, a fusion protein, a peptide, a SMIP, a small molecule, an oligonucleotide, an oligosaccharide, a soluble cytokine receptor or fragment thereof, a soluble TNF receptor Type I or a functional fragment thereof, a polypeptide that binds to TNF, and a dominant negative TNF molecule.

72. The method of claim 71, wherein the oligonucleotide is an siRNA.

73. The method of claim 71, wherein the direct TNF-I is selected from the group consisting of: Humira® (adalimumab/D2E7); Remicade® (infliximab); Cimzia® (CDP-870); Humicade® (CDP-570); golimumab (CNTO 148); CytoFab (Protherics); AME-527; anti-TNF-Receptor 1 mAb or dAb; ABX-10131; polyclonal anti-TNF antibodies; anti-TNF polyclonal anti-serum; anti-TNF or anti-TNF-R SMIPs (Trubion); Enbrel® (etanercept); pegsunercept/PEGs TNF-R1, onercept; recombinant TNF binding protein (r-TBP-1); trimerized TNF antagonist; SSR-150106 (Sanofi-Synthelabo); ABX-0402 (Ablynx); nanobody therapeutics (Ablynx); trimerized TNF antagonist (Borean); humanized anti-TNF mAb (Biovation); Dom-0200 (Domantis); Genz-29155 (Genzyme); agarooligosaccharide (Takara Shuzo); HTDN-TNF (Xencor); and therapeutic human polyclonal anti-TNF and anti-TNF-R antibodies (THP).

74. The method of claim 60, wherein the NFκB-I is selected from the group consisting of sulfasalazine, sulindac, clonidine, helenalin, wedelolactone, pyrollidinedithiocarbamate (PDTC), IKK-2 inhibitors, and IKK inhibitors.

75. The method of claim 59, wherein the administration comprises: (a) an induction regimen comprising a direct TNF-I; and (b) a maintenance regimen comprising a direct TNF-I.

76. The method of claim 60, wherein the administration comprises: (a) an induction regimen comprising an NFκB-I; and (b) a maintenance regimen comprising an NFκB-I.

77. The method of claim 75 or 76, wherein the induction regimen is administered intrathecally, intradiskally, peridiskally, or epidurally, or combinations thereof.

78. The method of claim 75 or 76, wherein the maintenance regimen comprises systemic or parenteral administration.

79. The method of claim 59, wherein the device implanted in the spinal device or fusion procedure is a source of a direct TNF-I.

80. The method of claim 60, wherein the device implanted in the spinal device or fusion procedure is a source of an NFκB-I.

81. The method of claim 59 or 79, further comprising administering an SAI.

82. The method of claim 60 or 80, further comprising administering an SAI.

83. The method of claim 81, wherein the implanted device is not a source of the SAI.

84. The method of claim 82, wherein the implanted device is not a source of the SAI.

85. The method of claim 81, wherein the SAI is selected from the group consisting of a second TAT, a corticosteroid, ozone, an antirheumatic drug, an LA, a neuroprotective agent, a salicylic acid acetate, a hydromorphone, a non-steroidal anti-inflammatory drug, a cox-2 inhibitor, an antidepressant, an anticonvulsant, a calcium channel blocker, and an antibiotic.

86. The method of claim 82, wherein the SAI is selected from the group consisting of a second TAT, a corticosteroid, ozone, an antirheumatic drug, an LA, a neuroprotective agent, a salicylic acid acetate, a hydromorphone, a non-steroidal anti-inflammatory drug, a cox-2 inhibitor, an antidepressant, an anticonvulsant, a calcium channel blocker, and an antibiotic.

87. A method for improving the outcome of a spinal device or fusion procedure in a subject, wherein the subject meets at least one predetermined SOE for a spinal device or fusion procedure, and wherein the spinal device or fusion procedure implants a device that is a source of a TAT, the method comprising:
a) optionally identifying the subject as a subject eligible for the spinal device or fusion procedure;
b) administering to the subject a therapeutically effective amount of at least one direct TNF-I that is in addition to the TAT derived from the implanted device; and
c) performing the spinal device or fusion procedure.

88. A method for improving the outcome of a spinal device or fusion procedure in a subject, wherein the subject meets at least one predetermined SOE for a spinal device or fusion procedure, and wherein the spinal device or fusion procedure implants a device that is a source of a TAT, the method comprising:
a) optionally identifying the subject as a subject eligible for the spinal device or fusion procedure;
b) administering to the subject a therapeutically effective amount of at least one NFκB-I that is in addition to the TAT derived from the implanted device; and
c) performing the spinal device or fusion procedure.

89. The method of claim 87 or 88, wherein the subject is:
a) eligible for a disk nucleus replacement procedure;
b) eligible for an annular repair procedure;
c) eligible for a dynamic stabilization procedure;
d) eligible for an artificial disk procedure;
e) eligible for an interbody spine fusion;
f) eligible for a posterolateral fusion;
g) eligible for an interbody spine fusion using BMP-2;
h) eligible for kyphoplasty, vertebroplasty or vertebral restoration;
i) eligible for facet replacement; or
j) eligible for spinal procedure involving implantation of an anti-adhesive device.

90. The method of claim 87 or 88, wherein the predetermined SOE is selected from:
a) a determination of eligibility of the subject for the spinal device or fusion procedure by a healthcare service provider, as evidenced by:
i) a scheduling or request for scheduling by a healthcare service provider of the spinal device or fusion procedure for the subject;
ii) a communication by a healthcare service provider to the subject that the subject has been determined to be eligible for the spinal device or fusion procedure;
iii) a provision or offering by a healthcare service provider to the subject of a consent form for the spinal device or fusion procedure;
iv) a receipt or execution by the subject of a consent form for the spinal device or fusion procedure, said consent form provided by the subject's healthcare provider; or
v) a notation by the healthcare service provider in a tangible medium that the patient is eligible for the spinal device or fusion procedure;
b) a determination of eligibility of the subject for the spinal device or fusion procedure by a qualified entity other than the subject's healthcare provide; and
c) the meeting by the subject of the eligibility criteria for a spinal device or fusion procedure in one or more CPG(s) or clinical trial(s).

91. The method of claim 87, wherein the direct TNF-I is selected from the group consisting of an antibody or antibody fragment, a fusion protein, a peptide, a SMIP, a small molecule, an oligonucleotide, an oligosaccharide, a soluble cytokine receptor or fragment thereof, a soluble TNF receptor Type I or a functional fragment thereof, a polypeptide that binds to TNF, and a dominant negative TNF molecule.

92. The method of claim 91, wherein the oligonucleotide is an siRNA.

93. The method of claim 91, wherein the direct TNF-I is selected from the group consisting of: Humira® (adalimumab/D2E7); Remicade® (infliximab); Cimzia® (CDP-870); Humicade® (CDP-570); golimumab (CNTO 148); CytoFab (Protherics); AME-527; anti-TNF-Receptor 1 mAb or dAb; ABX-10131; polyclonal anti-TNF antibodies; anti-TNF polyclonal anti-serum; anti-TNF or anti-TNF-R SMIPs (Trubion); Enbrel® (etanercept); pegsunercept/PEGs TNF-R1, onercept; recombinant TNF binding protein (r-TBP-1); trimerized TNF antagonist; SSR-150106 (Sanofi-Synthelabo); ABX-0402 (Ablynx); nanobody therapeutics (Ablynx); trimerized TNF antagonist (Borean); humanized anti-TNF mAb (Biovation); Dom-0200 (Domantis); Genz-29155 (Genzyme); agarooligosaccharide (Takara Shuzo); HTDN-TNF (Xencor); and therapeutic human polyclonal anti-TNF and anti-TNF-R antibodies (THP).

94. The method of claim 88, wherein the NFκB-I is selected from the group consisting of sulfasalazine, sulindac, clonidine, helenalin, wedelolactone, pyrollidinedithiocarbamate (PDTC), IKK-2 inhibitors, and IKK inhibitors.

95. The method of claim 87, wherein the administration comprises: (a) an induction regimen comprising a direct TNF-I; and (b) a maintenance regimen comprising a direct TNF-I.

96. The method of claim 88, wherein the administration comprises: (a) an induction regimen comprising an NFκB-I; and (b) a maintenance regimen comprising an NFκB-I.

97. The method of claim 95 or 96, wherein the induction regimen is administered intrathecally, intradiskally, peridiskally, or epidurally, or combinations thereof.

98. The method of claim 95 or 96, wherein the maintenance regimen comprises systemic or parenteral administration.

99. A kit comprising an implantable spinal device selected from the group consisting of a nucleus replacement device, an annular repair device; a dynamic stabilization device, an artificial disk, a fusion device, a kyphoplasty or vertebroplasty device, and a facet replacement device, and a direct TNF-I, wherein the direct TNF-I is
a) contained within or on the implantable spinal device;
b) contained in a vial;
c) disposed within a syringe, catheter, pump, or delivery device adapted for epidural, intradiskal, or peridiskal administration, or any combination thereof, or
d) disposed within a depot, hydrogel, or controlled-release formulation.

100. A kit comprising an implantable spinal device selected from the group consisting of a nucleus replacement device, an annular repair device; a dynamic stabilization device, an artificial disk, a fusion device, a kyphoplasty or vertebroplasty device, and a facet replacement device, and an NFκB-I, where the NFκB-I:
a) contained within or on the implantable spinal device;
b) contained in a vial;
c) disposed within a syringe, catheter, pump, or delivery device adapted for epidural, intradiskal, or peridiskal administration, or any combination thereof, or
d) disposed within a depot, hydrogel, or controlled-release formulation.

101. A kit comprising an implantable spinal device and a TNF-I, where the TNF-I is contained within a vial or is disposed within a syringe, catheter, pump, or delivery device adapted for epidural, intradiskal, or peridiskal administration, or any combination thereof.

102. A kit comprising an implantable spinal device and an NFκB-I, where the NFκB-I is contained within a vial or is disposed within a syringe, catheter, pump, or delivery device adapted for epidural, intradiskal, or peridiskal administration, or any combination thereof.

103. The kit of claim 99, 100, 101, or 102, further comprising an SAI.

104. An implantable spinal device selected from the group consisting of a nucleus replacement device, an annular repair device; a dynamic stabilization device, an artificial disk, a fusion device, a kyphoplasty or vertebroplasty device, and a facet replacement device, wherein the implantable spinal device comprises a TNF-I contained within or on the implantable spinal device.

105. An implantable spinal device selected from the group consisting of a nucleus replacement device, an annular repair device; a dynamic stabilization device, an artificial disk, a fusion device, a kyphoplasty or vertebroplasty device, and a facet replacement device, wherein the implantable spinal device comprises an NFκB-I contained within or on the implantable spinal device.
